SEQUENCE LISTING (132 sequences)

SEQ ID NO: 1 - DNA, 2563 bp, Homo sapiens
aggggagtctacaccctgtggagctcaagatggtcctgagtggggcgctgtgcttccgaatgaaggactcggcattgaaggtgctttatctgcataataaccagcttctagctggagggctgcatgcagggaaggtcattaaaggtgaagagatcagcgtggtccccaatcggtggctggatgccagcctgtcccccgtcatcctgggtgtccagggtggaagccagtgcctgtcatgtggggtggggcaggagccgactctaacactagagccagtgaacatcatggagctctatcttggtgccaaggaatccaagagcttcaccttctaccggcgggacatggggctcacctccagcttcgagtcggctgcctacccgggctggttcctgtgcacggtgcctgaagccgatcagcctgtcagactcacccagcttcccgagaatggtggctggaatgcccccatcacagacttctacttccagcagtgtgactagggcaacgtgcccccccagaactccctgggcagagccagctcgggtgaggggtgagtggaggagacccatggcggacaatcactctttctgctctcaggacccccaggtctgacttagtgggcacctgaccactttgtcttctggttcccagtttgcataaattctgagatttggagctcagtccagggtcctcccccactggatggtgctactgctgtggaaccttgtaaaaaccatgtggggtaaactgggaataacatgaaaagatttctgtgggggtggggtgggggagtgctgggaatcattcctgcttaatggtaactgacaagtgttaccctgagccccgcaggccaacccatccccagttgagccttatagggtcagtagctctccacatgaagtcctctcactcaccactgtgcaggagagggaggtggtcatagagtcagggatctatggcccttggcccagccccacccccttccctttatcctgccactgtcatatgctacctttcctatctcttccctcatcatcttgttgtgggcatgaggaggtggtgatgtcagaagaaatggttcgagctcagaagataaaagataagtagggtatgctgatcctcttttaaaaacccaagatacaatcaaaatcccagatgctggtctctattcccatgaaaaagtgctcatgacatattgagaagacctacttacaaagtggcatatattgcaatttattttaattaaaagatacctatttatatatttctttatagaaaaaagtctggaagagtttacttcaattgtagcaatgtcagggtggtggcagtataggtgatttttcttttaattctgttaatttatctgtatttcctaatttttctacaatgaagatgaattccttgtataaaaataagaaaagaaattaatcttgaggtaagcagagcagacatcatctctgattgtcctcagcctccaattccccagagtaaattcaaattgaatcgagctctgctgctctggttggttgtagtagtgatcaggaaacagatctcagcaaagccactgaggaggaggctgtgctgagtttgtgtggctggaatctctgggtaaggaacttaaagaacaaaaatcatctggtaattctttcctagaaggatcacagcccctgggattccaaggcattggatccagtctctaagaaggctgctgtactggttgaattgtgtccccctcaaattcacatccttcttggaatctcagtctgtgagtttatttggagataaggtctctgcagatgtagttagttaagacaaggtcatgctggatgaaggtagacctaaattcaatatgactggtttccttgtatgaaaaggagaggacacagagacagaggagacgcggggaagactatgtaaagatgaaggcagagatcggagttttgcagccacaagctaagaaacaccaaggattgtggcaaccatcagaagcttggaagaggcaaagaagaattcttccctagaggctttagagggataacggctctgctgaaaccttaatctcagacttccagcctcctgaacgaagaaagaataaatttcggctgttttaagccaccaaggataattggttacagcagctctaggaaactaatacagctgctaaaatgatccctgtctcctcgtgtttacattctgtgtgtgtcccctcccacaatgtaccaaagttgtctttgtgacccaatagaatatggcagaagtgatggcatgccacttccaagattaggttataaaagacactgcagcttctacttgagccctctctctctgccacccaccgcccccaatctatcttggctcactcgctctgggggaagctagctgccatgctatgagcaggcctataaagagacttacgtggtaaaaaatgaagtctcctgcccacagccacattagtgaacctagaagcagagactctgtgagataatcgatgtttgttgttttaagttgctcagttttggtctaacttgttatgcagcaatagataaataatatgcagagaaagaga

SEQ ID NO: 2 - DNA, 39 bp, Homo sapiens
ttgaggaacaggcagactccacagctcccgccaggagaa

SEQ ID NO: 3 - DNA, 42 bp, Homo sapiens
aaggaaggagggagaagggaaggagtgaaggaaggagtgaaa

SEQ ID NO: 4 - DNA, 1284 bp, Murine sp.
ggcacgaggggagcctgctttctacttaggtctcaaattttccagccttgtctttgcctaaaatttcctgctgtttatttcaaaatagggtctacatactgtggagctcatgatggttctgagtggggcactatgcttccgaatgaaggattcagccttgaaggtactgtatctgcacaataaccagctgctggctggaggactgcacgcagagaaggtcattaaaggtgaggagatcagtgttgtcccaaatcgggcactggatgccagtctgtcccctgtcatcctgggcgttcaaggaggaagccagtgcctatcttgtgggacagagaaagggccaattctgaaacttgagccagtgaacatcatggagctctacctcggggccaaggaatcaaagagcttcaccttctaccggcgggatatgggtcttacctccagcttcgaatccgctgcctacccaggctggttcctctgcacctcaccggaagctgaccagcctgtcaggctcactcagatccctgaggaccccgcctgggatgctcccatcacagacttctactttcagcagtgtgactagggctgcgtggtccccaaaactccataagcagaggcagagtaggcagtggcggctcctgatagaggatagagagacagaggagctccacagtaggtggcttactcctctccttccctactggactcccgcttctgacctaaggcacacagacactctcttctcctgcatcccagtgctggtaaatcttctggtatttggagctcaatgtgtagattctttcagattggatggtactacctctggtgtggaacccaatagaaaccacgtaggaccaacaaagagcaacataaaagattcttgggtgaagaagaggtgggaactgttcatacatagtaagatctgacacagtacctcagaagtcctgccattccttatgttctggagaaagtggagggggggtcaccaagactttctctggctggctgggccctttccctcaacctttctgacatctgcagcctctctcattcttgccttcattctctggccctgaaccgagagggtgatatcaggatagctgacagaagatgaccaggcacactgtcctggtttgaaaccagaggggacaataaaaaaccctgattctggtctctactcacataaaaagaagcttgtgaacattaagtgggaagagattgctactaaataacataccttgtaatttcatcttaattaaaatatacttctctatattatatatttta

SEQ ID NO: 5 - protein, 155 aa, Homo sapiens
MVLSGALCFRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQCD

SEQ ID NO: 6 - protein, 155 aa, Murine sp.
MVLSGALCFRMKDSALKVLYLHNNQLLAGGLHAEKVIKGEEISVVPNRALDASLSPVILGVQGGSQCLSCGTEKGPILKLEPVNIMELYLGAKESKSFTFYRRDMGLTSSFESAAYPGWFLCTSPEADQPVRLTQIPEDPAWDAPITDFYFQQCD

SEQ ID NO: 7 - protein, 141 aa, Artificial Sequence
  Description of Artificial Sequence Consensus polypeptide sequence
MVLSGALCFRMKDSALKVLYLHNNQLLAGGLHAKVIKGEEISVVPNRLDASLSPVILGVQGGSQCLSCGPLLEPVNIMELYLGAKESKSFTFYRRDMGLTSSFESAAYPGWFLCTPEADQPVRLTQPEWAPITDFYFQQCD

SEQ ID NO: 8 - protein, 138 aa, Homo sapiens
FRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQE

SEQ ID NO: 9 - protein, 73 aa, Artificial Sequence
  Description of Artificial Sequence Consensus polypeptide sequence
FRDKYLNNQLAGLVEIVVPPLGGGCLSCGELLEVNILKKFFRDGTSFESAAPGWFLCTEADQPVLTPGTFYFQ

SEQ ID NO: 10 - DNA, 465 bp, Homo sapiens
atggtcctgagtggggcgctgtgcttccgaatgaaggactcggcattgaaggtgctttatctgcataataaccagcttctagctggagggctgcatgcagggaaggtcattaaaggtgaagagatcagcgtggtccccaatcggtggctggatgccagcctgtcccccgtcatcctgggtgtccagggtggaagccagtgcctgtcatgtggggtggggcaggagccgactctaacactagagccagtgaacatcatggagctctatcttggtgccaaggaatccaagagcttcaccttctaccggcgggacatggggctcacctccagcttcgagtcggctgcctacccgggctggttcctgtgcacggtgcctgaagccgatcagcctgtcagactcacccagcttcccgagaatggtggctggaatgcccccatcacagacttctacttccagcagtgtgac

SEQ ID NO: 11 - DNA, 465 bp, Murine sp.
atggttctgagtggggcactatgcttccgaatgaaggattcagccttgaaggtactgtatctgcacaataaccagctgctggctggaggactgcacgcagagaaggtcattaaaggtgaggagatcagtgttgtcccaaatcgggcactggatgccagtctgtcccctgtcatcctgggcgttcaaggaggaagccagtgcctatcttgtgggacagagaaagggccaattctgaaacttgagccagtgaacatcatggagctctacctcggggccaaggaatcaaagagcttcaccttctaccggcgggatatgggtcttacctccagcttcgaatccgctgcctacccaggctggttcctctgcacctcaccggaagctgaccagcctgtcaggctcactcagatccctgaggaccccgcctgggatgctcccatcacagacttctactttcagcagtgtgac

SEQ ID NO: 12 - DNA, 41 bp, Artificial Sequence
  Description of Artificial Sequence Conserved consensus DNA sequence
acaatnaaaancccngatnctggtctctantcncatnaaaa

SEQ ID NO: 13 - protein, 155 aa, Artificial Sequence; identical to SEQ ID NO: 5
  Description of Artificial Sequence Recombinant IBR polypeptide

SEQ ID NO: 14 - protein, 154 aa, Artificial Sequence
  Description of Artificial Sequence Recombinant IBR polypeptide
VLSGALCFRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQCD

SEQ ID NO: 15 - protein, 157 aa, Artificial Sequence
  Description of Artificial Sequence Recombinant IBR polypeptide
GSSVLSGALCFRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQCD

SEQ ID NO: 16 - DNA, 73 bp, Homo sapiens
ctggcaatggcaggcaggaaagacagaggaaggaaggagggagaagggaaggagtgaaggaaggagtgaaaaa

SEQ ID NO: 17 - protein, 16 aa, Homo sapiens
MAGRKDRGRKEGEGKE

SEQ ID NO: 18 - DNA, 54 bp, Homo sapiens
ttggaggaacaggcagactccacagctcccgccaggagaaaggaacattctgag

SEQ ID NO: 19 - DNA, 10 bp, Homo sapiens
tccaaaatag

SEQ ID NO: 20 - DNA, 10 bp, Homo sapiens
gatgtttcag

SEQ ID NO: 21 - DNA, 10 bp, Homo sapiens
tttcccacag

SEQ ID NO: 22 - DNA, 10 bp, Homo sapiens
ctgccggcag

SEQ ID NO: 23 - DNA, 10 bp, Homo sapiens
ctggcaatgg

SEQ ID NO: 24 - DNA, 10 bp, Homo sapiens
ttggaggaac

SEQ ID NO: 25 - DNA, 10 bp, Homo sapiens
gggagtctac

SEQ ID NO: 26 - DNA, 10 bp, Homo sapiens
aatgaaggac

SEQ ID NO: 27 - DNA, 10 bp, Homo sapiens
gtgaagagat

SEQ ID NO: 28 - DNA, 10 bp, Homo sapiens
ccagtgaaca

SEQ ID NO: 29 - DNA, 10 bp, Homo sapiens
agtgaaaaag

SEQ ID NO: 30 - DNA, 10 bp, Homo sapiens
acattctgag

SEQ ID NO: 31 - DNA, 10 bp, Homo sapiens
tgtgcttccg

SEQ ID NO: 32 - DNA, 10 bp, Homo sapiens
gtcattaaag

SEQ ID NO: 33 - DNA, 10 bp, Homo sapiens
aacactagag

SEQ ID NO: 34 - DNA, 10 bp, Homo sapiens
agagaaagag

SEQ ID NO: 35 - DNA, 10 bp, Homo sapiens
gtaaggaaga

SEQ ID NO: 36 - DNA, 10 bp, Homo sapiens
gtatgctctg

SEQ ID NO: 37 - DNA, 10 bp, Homo sapiens
gtgagtgtat

SEQ ID NO: 38 - DNA, 10 bp, Homo sapiens
gttggtgatg

SEQ ID NO: 39 - DNA, 10 bp, Homo sapiens
gtgagacttg

SEQ ID NO: 40 - DNA, 10 bp, Homo sapiens
aaacaaatgc

SEQ ID NO: 41 - protein, 104 aa, Homo sapiens
MVLSGALCFRMKDSALKVLYLHNNQLLAGGLHAGKVIKGEEISVVPNRWLDASLSPVILGVQGGSQCLSCGVGQEPTLTLEVNIMELYLGAKESKSFTFYRRDM

SEQ ID NO: 42 - protein, 100 aa, Homo sapiens
RKSSKMQAFRIWDVNQKTFYLRNNQLVAGYLQGPNVNLEEKIDVVPIEPHALFLGIHGGKMCLSCVKSGDETRLQLEVNITDLSENRKQDKRFAFIRSDS

SEQ ID NO: 43 - DNA, 6540 bp, Homo sapiens
catgagcaaagatgttaatacaaagatgtttgtcacaacatggttttcaatagcaaaaaaagagagaaaaatatataaaagacaaataacagtggataggtttcaataaataatgttacagtgatacagttaaatactatacagctattgaagcatgtcattattcatatttagtatggaaagatattttgctattttgctacatgaaaaaatgaggttggaaaaagtataggttttgtgaatctgttgtatgaaagctgtctatagttacatgtgtatgtgtgtggaggaaaaagtgttgtcattggttttctgatgatgcactcagaaaagacaagtattcacattttttcttgtggctgatctggattttcaggtttttctacaatgaacatgtaggctgaacattccctaagcaggagagtcccacctctaacatctcctgtaggcctggcaatggcaggcaggaaagacagaggaaggaaggagggagaagggaaggagtgaaggaaggagtgaaaaaggtaaggaagaaagggaataggggaggaagggaggaaatgggaagggaaagaaggaaaggaaggaaagagggagggaagaaaggaagggaaaagggagggagtgagtgaatgaaagatggaaagaaggaagaaagggagggaggcagggaggaaagaaagttgcgcttcccttgagctgccatgggcactgactcttagggtctgaaagcccctgagatgcaaaagcctagtgctcacaaagagctggaaagcctcaaggaagttcttcaatatttctggaaggaaactgtctccagaagcttccctccccacgacagataatgagcagcaagtgcttctggcgacttagggtgatgtgaaatcacgctgggaatcctgctcctcctcaggtcctggcagtttcagggcccctccctaggccttacttaaaaggctgaggcatccttggaggaacaggcagactccacagctcccgccaggagaaaggaacattctgaggtatgctctggggcgctggtggtaccggagctctctcctgaccccagacccagaatctgctccgtggaggctgttcacatgctggggagctcggtgcagctgcttgctccccagaccccagccaactcagcctctctctccatgattttctgttgtttattccaaaataggggagtctacaccctgtggagctcaagatggtcctgagtggggcgctgtgcttccggtgagtgtatgaggccctggtttggtggtgtcctccggaggaagtgagttctggatagacccgttgtccagctctgagcaggagggaggaagggaggggctgccattgcagctgggaaattgtgaccagcacctcattgctcttagagttttcccagcctttttcaaataggggcaggactggggcaggccatctcacaaggggtccctgatgctgagggggacaagtgaacctcccagtctagagctccagccaagtctatccaaggtgggaacgggggccaggatccctgctcagagctccgccattgtcccccatcacagtgaatggatgtaagctcacccactctgtgcccctacctccctgctactctttggggataataataaaacaaaaaccattaccatcagccagtctgtccacccactggcatgtaccaagccagacactctgccgtgttctgggcttaacaacagaggatgagagtggtcctttctctcagtctaataaagcacttcccacgatgtgttctatgggactcgattagaggagtcccacagaggcatccaggagatgctttacacagtggagctctctgatcaagtaaatgcagggaattctgctttctacatcctctcataagagaaccacagcccagctcagcatatgagtgactgaggttttctgaagtaaggcaacttgttgaatcgtatttagctatgcatcgacccaatttttacactgcatccttttcccccatataacttttggagaaacccactttaggatacatcttccacctcataggatgccaggaaatcaactgagttcaaagatgagaaacaactttgaaaagttaaataaaagaaatttaaatttaaagaaactcctcacttagtaaggaatatatgaccaaatagaaatacatgtatcttgaagaattgaagaatcaggctttaacgtggaagaggcctggatgttatccaacccatcatcttagtgtagcaatggggaggctcaggcccagagtgggcgagagagttgtctcctgcgactcagcagcattggaggcatagatggggcaagaacctagggctctgactcaccgtgcagcttctcttccaacaggagatgggttggggcagaaaaggttgaatagggtgaaggagcaaaccacagactccagtgggagactgtggggtcatcctccttgtagggcatgagcccagcagggctgggagacaaggctgtgctgttacttctggcacagtaggaagaaagagagacaaaatgcctgagatcagggggttctctggatccagggcatgctggagtgtccaccctcctcctaatgtagtcctcaccccttcctgatgtttcagaatgaaggactcggcattgaaggtgctttatctgcataataaccagcttctagctggagggctgcatgcagggaaggtcattaaaggttggtgatgaaacatgacccactttccttggtctctatacactctcaggggagggggcctgaagagggcttagaatagtcatacagattagcataggcctacagagcccaggcattagggcagcacaaaccaggctctaagcaaaggcaaataaaatactacacctctcagcaaagtgaagacacacgctctggggccacctgaagcttctgtgcagaagtgagaatgttttccaagaggcttgtcttgtcattcccttacaggtagatttaggtcaagcattgcattccctgggagccagtaagtaccaaggagagaactaacgtagattctctataccttttttcccatatgggagtgggtttctgcctctccaccctgggtcccctctgctctctgaagatcctcagtcacttagagtggagggacccagagaacaggtggcattgttggacctcctgcttgctcactctgccccatgcactgcaacaggtccctctctaaaatagtttgcacctgcccacctggggcacccttgctgagcacagatgccaggtagatccttcagctaggccatatgtgtatgtgtgtgcttactggtgtatgtatgtgtgcatgcaggcatatatgtgtgagcatatgtgtgcatgcatgtatctgtatgtaaccatgtatgtgtgagtgcaggtatgtaggtatgagcatgtgtgtgtatatgtatatgtgtgcatgcatgtatctgtgcatgtatgtatctgatgtatgtgggtggtgaggggatgtacagagaggaatgagaccctcttttgctctcagcaacctcacagggtgtagaaagttgtccaaacaattccaaaggggggcttattaagacagggttcagaaaaaggcctgagacccaaggggcattaaaggagggggttgagtctattttgggttgtagaggcttgaagatttgaccctgaactagagggtggagtggaggtggtacaatgtgcttccatgccttgatgtccactctgggccagtggacaggagaagccatgtcatgacagctgctgagaagcctcccttctgcccagcctgggggcaggccgtctcacagcagtcctgtgccctagagcccaggacaggggaagaaggagggaaaggcatccagggccctgcatctggcctctttcccacaggtgaagagatcagcgtggtccccaatcggtggctggatgccagcctgtcccccgtcatcctgggtgtccagggtggaagccagtgcctgtcatgtggggtggggcaggagccgactctaacactagaggtgagacttggggcatcctcactggggactcagccacagatgctgagcctactgaagccgggcagcccacagccctggtgctgtgggacaccctagcaggattctgttgatggcagctttgcctcctccctaaggatcctgcccagccctccctctgcccctgcttctgccctcacctgacctcccctcctctgccggcagccagtgaacatcatggagctctatcttggtgccaaggaatccaagagcttcaccttctaccggcgggacatggggctcacctccagcttcgagtcggctgcctacccgggctggttcctgtgcacggtgcctgaagccgatcagcctgtcagactcacccagcttcccgagaatggtggctggaatgcccccatcacagacttctacttccagcagtgtgactagggcaacgtgccccccagaactccctgggcagagccagctcgggtgaggggtgagtggaggagacccatggcggacaatcactctctctgctctcaggacccccacgtctgacttagtgggcacctgaccactttgtcttctggttcccagtttggataaattctgagatttggagctcagtccacggtcctcccccactggatggtgctactgctgtggaaccttgtaaaaaccatgtggggtaaactgggaataacatgaaaagatttctgtgggggtggggtgggggagtggtgggaatcattcctgcttaatggtaactgacaagtgttaccctgagccccgcaggccaacccatccccagttgagccttatagggtcagtagctctccacatgaagtcctgtcactcaccactgtgcaggagagggaggtggtcatagagtcagggatctatggcccttggcccagccccacccccttccctttaatcctgccactgtcatatgctacctttcctatctcttccctcatcatcttgttgtgggcatgaggaggtggtgatgtcagaagaaatggctcgagctcagaagataaaagataagtagggtatgctgatcctcttttaaaaacccaagatacaatcaaaatcccagatgctggtctctattcccatgaaaaagtgctcatgacatattgagaagacctacttacaaagtggcatatattgcaatttattttaattaaaagatacctatttatatatttctttatagaaaaaagtctggaagagtttacttcaattgtagcaatgtcagggtggtggcagtataggtgatttttcttttaattctgttaatttatctgtatttcctaatttttctacaatgaagatgaattccttgtataaaaataagaaaagaaattaatcttgaggtaagcagagcagacatcatctctgattgtcctcagcctccacttccccagagtaaattcaaattgaatcgagctctgctgctctggttggttgtagtagtgatcaggaaacagatctcagcaaagccactgaggaggaggctgtgatgagtttgtgtggctggaatctctgggtaaggaacttaaagaacaaaaatcatctggtaattctttcctagaaggatcacagcccctgggattccaaggcattggatccagtctctaagaaggctgctgtactggttgaattgtgtccccctcaaattcacatccttcttggaatctcagtctgtgagtttatttggagataaggtctctgcagatgtagttagttaagacaaggtcatgctggatgaaggtagacctaaattcaatatgactggtttccttgtatgaaaaggagaggacacagagacagaggagacgcggggaagactatgtaaagatgaaggcagagatcggagttttgcagccacaagctaagaaacaccaaggattgtggcaaccatcagaagcttggaagaggcaaagaagaattcttccctagaggctttagagggataacggctctgctgaaaccttaatctcagacttccagcctcctgaacgaagaaagaataaatttcggctgttttaagccaccaaggataattggttatggcagctctaggaaactaatacagctgctaaaatgatccctgtctcctcgtgtttacattctgtgtgtgtcccctcccacaatgtaccaaagttgtctttgtgaccaatagaatatggcagaagtgatggcatgccacttccaagattaggttataaaagacactgcagcttctacttgagccctctctctctgccacccaccgcccccaatctatcttggctcactcgctctgggggaagctagcttccatgctatgagcaggcctataaagagacttatgtggtaaaaaatgaagtctcctgcccacagccacattagtgaacctagaagcagagactctgtgagataatcaatgtttgttgttttaagttgctcagttttggtctaacttgttatgcagcaatagataaataatatgcagagaaagagaaacaaatgcatttgttt

SEQ ID NO: 44 - protein, 152 aa, Homo sapiens
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 45 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 46 - protein, 159 aa, Homo sapiens
SAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 47 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 48 - protein, 6 aa, Artificial Sequence
  Description of Artificial Sequence Consensus peptide sequence
LKXLXL

SEQ ID NO: 49 - protein, 7 aa, Artificial Sequence
  Description of Artificial Sequence Consensus peptide sequence
ITDFXXQ

SEQ ID NO: 50 - protein, 12 aa, Artificial Sequence
  Description of Artificial Sequence Consensus peptide sequence
YLXNNQLXAGXL

SEQ ID NO: 51 - protein, 9 aa, Artificial Sequence
  Description of Artificial Sequence Consensus peptide sequence
LEXVNIXXL

SEQ ID NO: 52 - protein, 24 aa, Artificial Sequence
  Description of Artificial Sequence Consensus peptide sequence
TXSFESAAXPGWFLCTXXEADQPV

SEQ ID NO: 53 - protein, 4 aa, Artificial Sequence
  Description of Artificial Sequence Binding domain
FGFR

SEQ ID NO: 54 - protein, 13 aa, Artificial Sequence
  Description of Artificial Sequence N-terminal extension
GSSGLRRASLGSS

SEQ ID NO: 55 - DNA, 485 bp, Homo sapiens
tttttttctataaagaaatatatgggtaggttttttttaattaaaataaattgcaatatatgccactttgtaagtaggtcttctcaatatgtcatgagcactttttcatgggaatagagaccagcatctgggattttgattgtatcttgggtttttaaaagaggatcagcataccctacttatcttttatcttctgagctcgagccatttcttctgacatcagcacctcctcatgcccacaacaagatgatgagggaagagataggaaaggtagcatatgacagtggcaggattaaagggaaggaggtggggctgggccaagggccatagatccctgactctatgaccacctccctctcctgcatagtggtgagtgacaggtcttcatgtggagagctactgaccctataaggctcaactggggatgggttggcctgcggggctcaaggtaacactggtcagttaccattaagcaggaatgattc

SEQ ID NO: 56 - DNA, 442 bp, Homo sapiens
ttttttttttgtttctctttctctgcatattatttatctattgctgcataacaagttagaccaaaactgagcaacttaaaacaacaaacatcgattatctcacagagtctctgcttctaggttcactaatgtggctgtgggcaggagacttcattttttaccacgtaagtctctttataggcctgctcatagcatggcagctagcttcccccagagcgagtgagccaagatagattgggggcggtgggtggcagagagagagggctcaagtagaagctgcagtgtcttttataacctaatcttggaagtggcatgccatcacttctgccatattctattggtcacaaagacaactttggtacattgtgggaggggacacacacagaatgtaaacacgaggagacagggatcattttagcagctgtattagtttcctagagct

SEQ ID NO: 57 - DNA, 414 bp, Homo sapiens
ctctgcatattatttatctattgctgcataacaagttagaccaaaactgagcaacttaaaacaacaaacattgattatctcacagagtctctgcttctaggttcactaatgtggctgtgggcaggagacttcattttttaccacataagtctctttataggcctgctcatagcatggaagctagcttcccccagagcgagtgagccaagatagattgggggcggtgggtggcagagagagagggctcaagtagaagctgcagtgtcttttataacctaatcttggaagtggcatgccatcacttctgccatattctattggtcacaaagacaactttggtacattgtgggaggggacacacacagaatgtaaacacgaggagacagggatcattttagcagctgtattagtttc

SEQ ID NO: 58 - DNA, 410 bp, Homo sapiens
tttttttctataaagaaatatataaataggtatcttttaattaaaataaattgcaatatatgccactttgtaagtaggtcttctcaatatgtcatgagcactttttcatgggaatagagaccagcatctgggattttgattgtatcttgggtttttaaaagaggatcagcataccctacttatcttttatcttctgagctcgagccatttcttctgacatcagcacctcctcatgcccacaacaagatgatgagggaagagataggaaaggtagcatatgacagtggcaggattaaagggaaggaggtggggctgggccaagggccatagatccctgactctatgaccacctccctctcctgcatagtggtgagtgacaggtcttcatgtggagagctactgaccctata

SEQ ID NO: 59 - DNA, 374 bp, Homo sapiens
  misc_feature (7)..(8): n is a, c, g, t or u
tctcttnntnattcatattatttatctattgctgcataacaagttagaccaaaactgagcaacttaaaacaacaaacatcgattatctcacagagtctctgcttctaggttcactaatgtggctgtgggcaggagacttcattttttaccacgtaagtctctttataggcctgctcatagcatggcagctagcttcccccagagcgagtgagccaagatagattgggggcggtgggtggcagagagagagggctcaagtagaagctgcagtgtcttttataacctaatcttggaagtggcatgccatcacttctgccatattctattgggtcacaaagacaactttgggtacattgtgggaggggacacacaca

SEQ ID NO: 60 - DNA, 348 bp, Homo sapiens
  misc_feature (343)..(343): n is a, c, g, t or u
ggtagacctaaattcaatatgactggtttccttgtatgaaaaggagaggacacagagacagaggagacgcggggaagactatgtaaagatgaaggcagagatcggagttttgcagccacaagctaagaaacaccaaggattgtggcaaccatcagaagcttggaagaggcaaagaagaattcttccctagaggctttagagggataacggctctgctgaaaccttaatctcagacttccagcctcctgaacgaagaaagaataaatttcggctgttttaagccaccaaggataattggttacagcagctctagggaaactaattacagctggttaaaatggtnccctt

SEQ ID NO: 61 - DNA, 382 bp, Mus musculus
ggagcctgctttctacttaggtctcaaattttccagccttgtctttgcctaaaatttcctgctgtttatttcaaaatagggtctacatactgtggagctcatgatggttctgagtggggcactatgcttccgaatgaaggattcagccttgaaggtactgtatctgcacaataaccagctgctggctggaggactgcacgcagagaaggtcattaaaggtgaggagatcagtgttgtcccaaatcgggcactggatgccagtctgtcccctgtcatcctgggcgttcaaggaggaagccagtgcctatcttgtgggacagagaaagggccaattctgaaacttgagccagtgaacatcatggagctctacctcggggccaag

SEQ ID NO: 62 - DNA, 554 bp, Mus musculus
cagcctgtcaggctcactcagatccctgaggaccccgcctgggatgctcccatcacagacttctactttcagcagtgtgactagggctgcgtggtccccaaaactccataagcagaggcagagtaggcagtggcggctcctgatagaggatagagagacagaggagctccacagtaggtggcttactcctctccttccctactggactcccgcttctgacctaaggcacacagacactctcttctcctgcatcccagtgctggtaaatcttctggtatttggagctcaatgtgtagattctttcagattggatggtactacctctggtgtggaacccaatagaaaccacgtaggaccaacaaagagcaacataaaagattcttgggtgaagaagaggtgggaactgttcatacatagtaagatctgacacagtacctcagaagtcctgccattccttatgttctggagaaaggggaggggggggtcaccaagactttctctggctggctgggccctttccctcaacctttctgacatctgcagcctctctcatt

SEQ ID NO: 63 - DNA, 354 bp, Mus musculus
ttcggcacaggcagcctgtcaggtcactcagatccctgaggaccccgcctgggatgctcccatcacagacttctactttcagcagtgtgactagggctgcgtggtccccaaaactccataagcagaggcagagtaggcagtggcggctcctgatagaggatagagagacagaggagctccacagtaggtggcttactcctctccttccctactggactcccgcttctgacctaaggcacacagacactctcttctcctgcatcccagtgctggtaaatcttctggtatttggagctcaatgtgtagattctttcagattggatggtactacctctggtgtggaaccccaataga

SEQ ID NO: 64 - DNA, 477 bp, Homo sapiens
ttttttaaaacaacaaatatttattatgtcacccagtttccaagggtcaggaatccaggagcagtttaacagggttgtcctagctcaaagtcacccttgagagtgtagtcaaactgtcagccaggggtgtaggcatctgaaggcatgactggggctggagaaagcccaagcagttggagaacagcctccttcacaaggtggttggcagttctcagtttctcattagctgttggtcacaaggtggacctcttcatagggctgttcatgacatagcagctgacttgtcccagagtatgatccaagagagacagacaggaagacaagcagacagacagacaccaagatcgaagctgcagtatgctttgtaacctaatcttggaagtgacatgccatattctaccatattctgttggtcacatagactaaccttggtacagtgtgggaggggactttacagggatataaattccataaagt

SEQ ID NO: 65 - DNA, 21 bp, Artificial Sequence
  Chemically synthesized primer
tggtctctcatttgtctcatc

SEQ ID NO: 66 - DNA, 20 bp, Artificial sequence
  Chemically synthesized primer
ccaagatgaagaccaaccag

SEQ ID NO: 67 - DNA, 25 bp, Artificial Sequence
  Chemically synthesized primer
ctttaataatagtaaccaggcaaga

SEQ ID NO: 68 - DNA, 22 bp, Artificial sequence
  Chemically synthesized primer
gtaaataggctgaaaggggaaa

SEQ ID NO: 69 - DNA, 20 bp, Artificial sequence
  Chemically synthesized primer
gtatatgctcaggtgtcctc

SEQ ID NO: 70 - DNA, 25 bp, Artificial sequence
  Chemically synthesized primer
acttaagtttaggaatcttcccact

SEQ ID NO: 71 - DNA, 30 bp, Artificial sequence
  Chemically synthesized primer
acgttggatggcattagcctcatccttgag

SEQ ID NO: 72 - DNA, 30 bp, Artificial sequence
  Chemically synthesized primer
acgttggatgtcttttatagggctcaggtg

SEQ ID NO: 73 - DNA, 20 bp, Artificial sequence
  Chemically synthesized primer
agggctcactcgaatgactt

SEQ ID NO: 74 - DNA, 30 bp, Artificial sequence
  Chemically synthesized primer
acgttggatgagccagattgcaaagactag

SEQ ID NO: 75 - DNA, 30 bp, Artificial sequence
  Chemically synthesized primer
acgttggatgtctagagtccattgaggtgg

SEQ ID NO: 76 - DNA, 30 bp, Artificial sequence
  Chemically synthesized primer
acgttggatgagcttctcagagccactgtg

SEQ ID NO: 77 - DNA, 30 bp, Artificial sequence
  Chemically synthesized primer
acgttggatgcacacccgatgattgatatc

SEQ ID NO: 78 - DNA, 30 bp, Artificial sequence
  Chemically synthesized primer
acgttggatggtggaaaagaggcttgttag

SEQ ID NO: 79 - DNA, 20 bp, Artificial sequence
  Chemically synthesized primer
ggactgacagtggagcaaca

SEQ ID NO: 80 - DNA, 30 bp, Artificial sequence
  Chemically synthesized primer
acgttggatgctacactgtgcaacttcagc

SEQ ID NO: 81 - DNA, 30 bp, Artificial sequence
  Chemically synthesized primer
acgttggatgaagaaggaagaaagggaggg

SEQ ID NO: 82 - DNA, 20 bp, Artificial sequence
  Chemically synthesized primer
ggaaattgtgaccagcacct

SEQ ID NO: 83 - DNA, 25 bp, Artificial sequence
  Chemically synthesized primer
gtaagctcacccactctgtgcccgt

SEQ ID NO: 84 - DNA, 30 bp, Artificial sequence
  Chemically synthesized primer
acgttggatggtgggcaggtgcaaactatt

SEQ ID NO: 85 - DNA, 30 bp, Artificial sequence
  Chemically synthesized primer
acgttggatggtctctttataggcctgctc

SEQ ID NO: 86 - DNA, 30 bp, Artificial sequence
  Chemically synthesized primer
acgttggatggaatctctggatcttgctcc

SEQ ID NO: 87 - DNA, 20 bp, Artificial sequence
  Chemically synthesized primer
caaggtggtgattcagagca

SEQ ID NO: 88 - DNA, 29 bp, Artificial sequence
  Chemically synthesized primer
ttctatctgaggaacaaccaactagtagc

SEQ ID NO: 89 - DNA, 20 bp, Artificial sequence
  Chemically synthesized primer
acacaggaaggtgccaagca

SEQ ID NO: 90 - DNA, 17 bp, Artificial sequence
  Chemically synthesized primer
ctcagcaacactcctat

SEQ ID NO: 91 - DNA, 20 bp, Artificial sequence
  Chemically synthesized primer
ttgtggggaccaggggagat

SEQ ID NO: 92 - DNA, 20 bp, Artificial sequence
  Chemically synthesized primer
agggaggcagcacaggactt

SEQ ID NO: 93 - DNA, 21 bp, Artificial sequence
  Chemically synthesized primer
tgttgcttgctcagtgttttg

SEQ ID NO: 94 - DNA, 20 bp, Artificial sequence
  Chemically synthesized primer
aggtcaggcagggaaaggaa

SEQ ID NO: 95 - DNA, 20 bp, Artificial sequence
  Chemically synthesized primer
gcccaaggtgtgtcttctgt

SEQ ID NO: 96 - DNA, 22 bp, Artificial sequence
  Chemically synthesized primer
gtaaataggctgaaaggggaaa

SEQ ID NO: 97 - DNA, 20 bp, Artificial sequence
  Chemically synthesized primer
catggagaattagcaagctg

SEQ ID NO: 98 - DNA, 20 bp, Artificial sequence
  Chemically synthesized primer
gagcttatctccagggttgc

SEQ ID NO: 99 - DNA, 30 bp, Artificial sequence
  Chemically synthesized primer
acgttggatgaaactcccctttagagaccc

SEQ ID NO: 100 - DNA, 30 bp, Artificial sequence
  Chemically synthesized primer
acgttggatgaattgcaggaggtgcagatg

SEQ ID NO: 101 - DNA, 20 bp, Artificial sequence
  Chemically synthesized primer
atggggtaggaggagtgacc

SEQ ID NO: 102 - DNA, 30 bp, Artificial sequence
  Chemically synthesized primer
acgttggatgggtgaagtgaacattccctg

SEQ ID NO: 103 - DNA, 30 bp, Artificial sequence
  Chemically synthesized primer
acgttggatgatgctgcttctccatggaag

SEQ ID NO: 104 - DNA, 30 bp, Artificial sequence
  Chemically synthesized primer
acgttggatgtcaggagctggctttgctac

SEQ ID NO: 105 - DNA, 30 bp, Artificial sequence
  Chemically synthesized primer
acgttggatgtgactttctcaacagcattg

SEQ ID NO: 106 - DNA, 30 bp, Artificial sequence
  Chemically synthesized primer
acgttggatgctctcctgttacacactcag

SEQ ID NO: 107 - DNA, 20 bp, Artificial sequence
  Chemically synthesized primer
ggcctaacatcagatgcaaa

SEQ ID NO: 108 - DNA, 30 bp, Artificial sequence
  Chemically synthesized primer
acgttggatggggaaaatcatctgcatggg

SEQ ID NO: 109 - DNA, 30 bp, Artificial sequence
  Chemically synthesized primer
acgttggatgtcagaccctaagagtcagtg

SEQ ID NO: 110 - DNA, 25 bp, Artificial sequence
  Chemically synthesized primer
gagctctagactgggaggttcaatt

SEQ ID NO: 111 - DNA, 18 bp, Artificial sequence
  Chemically synthesized primer
acatgccagtgggtggac

SEQ ID NO: 112 - DNA, 30 bp, Artificial sequence
  Chemically synthesized primer
acgttggatgattgttggacctcctgcttg

SEQ ID NO: 113 - DNA, 30 bp, Artificial sequence
  Chemically synthesized primer
acgttggatgcccaatctatcttggctcac

SEQ ID NO: 114 - DNA, 30 bp, Artificial sequence
  Chemically synthesized primer
acgttggatgccttcatcattctcctgcac

SEQ ID NO: 115 - DNA, 20 bp, Artificial sequence
  Chemically synthesized primer
gggaggaaagggaagaagag

SEQ ID NO: 116 - DNA, 25 bp, Artificial sequence
  Chemically synthesized primer
caccagacttgacacaggacaggca

SEQ ID NO: 117 - DNA, 20 bp, Artificial sequence
  Chemically synthesized primer
tgcagacagacgggcaaagt

SEQ ID NO: 118 - DNA, 17 bp, Artificial sequence
  Chemically synthesized primer
tcctggtctgcaggtaa

SEQ ID NO: 119 - DNA, 20 bp, Artificial sequence
  Chemically synthesized primer
agcctggcactctgctgaat

SEQ ID NO: 120 - DNA, 20 bp, Artificial sequence
  Chemically synthesized primer
agtccctgcagtccttgcca

SEQ ID NO: 121 - DNA, 20 bp, Artificial sequence
  Chemically synthesized primer
ctctgcggagaaaggaagtc

SEQ ID NO: 122 - DNA, 17 bp, Artificial sequence
  Chemically synthesized primer
gggctcaggtgggctcc

SEQ ID NO: 123 - DNA, 22 bp, Artificial sequence
  Chemically synthesized primer
tgcatggacatctacaaatggc

SEQ ID NO: 124 - DNA, 21 bp, Artificial sequence
  Chemically synthesized primer
gagctggctttgctacagtaa

SEQ ID NO: 125 - DNA, 19 bp, Artificial sequence
  Chemically synthesized primer
gcatgctgcttctccatgg

SEQ ID NO: 126 - DNA, 20 bp, Artificial sequence
  Chemically synthesized primer
tgatatcctgaatgctcccc

SEQ ID NO: 127 - DNA, 20 bp, Artificial sequence
  Chemically synthesized primer
gaggcttgttagagaggagg

SEQ ID NO: 128 - DNA, 19 bp, Artificial sequence
  Chemically synthesized primer
cttcagcctgcccacctta

SEQ ID NO: 129 - DNA, 21 bp, Artificial sequence
  Chemically synthesized primer
gaggcagggaggaaagaaagt

SEQ ID NO: 130 - DNA, 23 bp, Artificial sequence
  Chemically synthesized primer
tgcaaactattttagagagggac

SEQ ID NO: 131 - DNA, 19 bp, Artificial sequence
  Chemically synthesized primer
aggcctgctcatagcatgg

SEQ ID NO: 132 - DNA, 21 bp, Artificial sequence
  Chemically synthesized primer
tctggatcttgctccacagaa

(19) United States
(12) Patent Application Publication
(10) Pub. No.: US 2006/0183161 A1
(43) Pub. Date: Aug. 17, 2006

(54) IL-ILI gene and polypeptide products

(76) Inventors: Martin Nicklin, (US); Jenny Barton, (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/203,682

(22) Filed: Aug. 12, 2005

Related U.S. Application Data
(63) Continuation of application No. 09/617,720, filed on Jul. 17, 2000.

(51) Int. Cl. C07K 14/545; G01N 33/567; C07H 21/04; C12P 21/02

(52) U.S. Cl. 435/7.2; 530/351; 435/69.5; 435/320.1; 435/325; 536/23.5

(57) ABSTRACT

The present invention relates to the discovery of novel genes encoded by the interleukin-1 locus. The IL-1L1 gene is located in the interval between the IL-1Beta and the IL-1 receptor antagonist encoding genes and encodes a polypeptide which is homologous to both IL-1 agonist and IL-1 antagonist proteins. Therapeutics, diagnostics and screening assays based on these molecules are also disclosed.

### RELATED APPLICATIONS

This application is a continuation-in-part application of U.S. application Ser. No. 09/617,720, filed Jul. 17, 2000; the entire contents of this application is incorporated herein by reference.

### 1. BACKGROUND OF THE INVENTION

The interleukin-1 gene cluster encodes a number of related polypeptides that regulate the inflammatory response. The IL-1 gene cluster is located on the long arm of human chromosome 2 (2q13) and contains the genes for at least three interleukin-1 signaling ligands including IL-1a (encoded by IL-1A), IL-1b (encoded by IL-1B), and the IL-1 receptor antagonist (IL-1ra encoded by IL-1RN) within a region of 430 kb (Nicklin, et al. (1994) Genomics 19: 382-4). The agonist molecules, IL-1a and IL-1b, have potent pro-inflammatory activity and initiate many inflammatory cascades. For example, the IL-1a and IL-1b cytokines stimulate production of still other cytokines, such as IL-6 and IL-8, and thereby trigger the activation and recruitment of leukocytes into damaged tissue as well as the local production of vasoactive agents. Inappropriate production of IL-1 agonist activity appears to play a central role in the pathology of autoimmune and inflammatory diseases including rheumatoid arthritis, inflammatory bowel disorder, and psoriasis. In contrast the IL-1 receptor antagonist (IL-1ra) prevents signal transduction of IL-1 agonists by competitively binding to certain IL-1 receptors without inducing an inflammatory cascade. Deficiencies in IL-1 receptor antagonist activity may therefore also lead to autoimmune and inflammatory disease pathologies and such IL-1 receptor antagonists may provide useful reagents for the treatment of these diseases and conditions.

The IL-1 agonists act through at least two types of receptors. Type I IL-1 receptors (IL-1RI) transduce a signal to the interior of the cell, while type II receptors (IL-1RII) do not and are therefore sometimes known as “decoy” receptors. The IL-1 responsiveness of a cell is affected by the ratio between IL-1RI and IL-1RII receptors, which appear to compete for interaction with a third membrane protein called IL-1 receptor accessory protein (IL-1RAcP) which facilitates IL-1-dependent activation of IL-1RI (Lang et al. (1998) J Immunol. 161:6871-7). IL-1 type I receptors are expressed on T cells, fibroblasts, and epithelial cells (Sims et al. (1988) Science 241: 585-9) while IL-1 type II receptors are expressed on B cells, neutrophils, and macrophages (Bomsztyk et al. (1989) Proc. Natl. Acad. Sci. USA 86: 8034-8). The IL-1a and IL-1b ligands both bind to type I and to type II IL-1 receptors and their biological activities as cytokines appear to be similar, however the apparent avidity of IL-1a is highest for IL-1RI while the apparent avidity of IL-1b is highest for IL-1RII.

The IL-1 receptor antagonist is structurally homologous to IL-1a and IL-1b and binds to IL-1 type I receptors but is biologically inactive, so that it functions as a competitive inhibitor of IL-1. In certain cell types, IL-1 receptor antagonist mRNA is alternatively spliced to incorporate two different first exons. One of the alternative spliced forms is secreted while the other isoform remains in the cytoplasm, but the function of this intracellular form of IL-1 receptor antagonist is not known. Hepatocytes produce the secreted, but not the cytoplasmic, form of IL-1ra in response to pro-inflammatory cytokines as an acute-phase protein (Gabay et al. (1997) J. Clin. Invest. 99: 2930-40). The use of IL-1 receptor antagonists has been suggested for the treatment of a number of inflammatory diseases and conditions (U.S. Patents

Genetic diversity of the IL-1RN gene may contribute to the etiology of certain inflammatory diseases and conditions. Intron 2 of the human IL-1RN gene encompasses a polymorphism caused by a variable number of tandem repeats (VNTR) of an 86 bp sequence (Tarlow, et al. (1993) Hum. Genet. 91: 403-4). At least 5 alleles of this polymorphism exist in human populations and a particular allele of this IL-1RN VNTR polymorphism (i.e. IL-1RN VNTR allele 2) has been associated with a more severe clinical outcome in several diseases having an inflammatory component including periodontal disease (U.S. Pat. No. 5,686,246), osteoporosis (U.S. Pat. No. 5,698,399), lupus erythematosus (Blakemore et al. (1994) Arthritis Rheum. 37: 1380-5), ulcerative colitis (Mansfield et al. (1994) Gastroenterology 106: 637-42) and alopecia areata (Tarlow et al. (1993) Hum. Genet. 91: 403-4) as well as diabetic nephropathy (see WO 98/15653) Blakemore et al. (1996) Hum. Genet. 97: 369-74). Other IL-1 polymorphic alleles linked to the IL-1RN (VNTR) have also been found associated with various inflammatory diseases or conditions such as chronic obstructive airway disease (WO 99/24615) and coronary artery disease (WO 98/40517). Interleukin-1ra levels are elevated in the blood of patients with a variety of infectious, autoimmune and traumatic conditions. Furthermore, a human haplotype of the IL-1 locus which is associated with an increased risk for inflammatory disease has been characterized (WO/98/54359).

IL-1a, IL-1b, IL-1ra and IL-18 are the only IL-1-like cytokines whose activities have so far been reported. Both forms of IL-1 (IL-1a and IL-1b, encoded by human genes IL1A and IL1B respectively) are single domain proteins that function as inflammatory cytokines through their interaction with the type I IL-1 receptor (IL-1R1). Complexes between IL-1 and IL-1RI recruit IL-1 receptor accessory protein (IL-1RAcP), a homolog of IL-1RI, and activate signal transduction in a process that involves the Toll-like domains of IL-1RI and IL-1RAcP (Korherr, et al., Eur. J. Immunol. (1997) 27:262-2671). The extracellular domains of both receptor components contain three immunoglobulin-like domains.

IL-1 receptor antagonist (IL-1ra, encoded by human gene IL1RN) is 28% identical to IL-1b but functions as an antagonist, blocking IL-1 signaling by competing with the IL-1 for association with IL-1R1. Complexes between IL-1R1 and IL-1ra do not recruit IL-1RAcP and so fail to activate signaling (Greenfeder, et al. (1995) J. Biol. Chem. 270: 13757-13765). IL-18 is also a homolog of IL-1, but interacts specifically with a different pair of receptors, IL-18R1 and AcPL (Torigoe, et al., (1997) J. Biol. Chem. 272: 25737-25742; Born, et al. (1998) J. Biol. Chem. 1998. 273: 29445-29446). Both IL-18 receptor components have the same domain structures as IL-1R1 and signal through Toll-like domains. Where expressed in similar cells, the signaling cascades downstream of the IL-1 and IL-18 receptors are similar (Thomassen, et al. (1998) J. Interferon Cytokine Res. 18: 1077-1088). Three other IL-1R1-like receptors, T1/ST2 (IL-1RL1) (Bergers, et al. (1994) EMBO J. 13: 1176-1188.), IL1R-rp2 (IL-1RL2) (Lovenberg, et al. (1996) Neuroimmunol. 70: 113-122) and IL1RAPL (Carrie, et al. (1999) Nat. Genet. 23: 25-31) have no known signal-evoking ligands, yet the cytoplasmic domain of ST2 has been shown to deliver an IL-1R1-like signal in transfected cells (Reikerstorfer, et al. (1995) J. Biol. Chem. 28: 17645-17648). We have previously identified a ˜430 kb human genomic fragment (the IL-1 cluster) (Reikerstorfer, et al. (1995) J. Biol. Chem. 28: 17645-17648.) from chromosome 2q.13 that contains IL-1a, IL-1b and IL-1ra (FIG. 8(a)). IL-18 maps elsewhere (Nolan, et al. (1998) Genomics 51: 161-163).

### 2. SUMMARY OF THE INVENTION

The present invention is based, at least in part, on the discovery of novel human and murine genes encoding novel proteins, which have sequence homologies with the interleukin-1 receptor antagonist protein (IL-1ra) as well as interleukin-1 (IL-1). The newly identified proteins and nucleic acids described herein are referred to as “IL-1L1s” and are exemplified here by both human and murine homologs of this gene. The human IL-1L1 gene (herein referred to as hIL-1L1) transcript is shown in FIG. 1 and includes a 2562 nucleotide common sequence (SEQ ID No. 1) and two alternative 5′ ends of 39 nucleotides (SEQ ID No. 2) and 42 nucleotides (SEQ ID No. 3). The hIL-1L1transcript includes 5′ and 3′ untranslated regions and a 465 nucleotide open reading frame (SEQ ID No. 4) encoding a 155 amino acid hIL-1L1polypeptide shown in FIG. 3A (SEQ ID No. 5). The hIL-1L1gene is highly expressed in placental and, to a lesser extent, in thymus tissues (FIG. 7).A nucleic acid comprising the cDNA encoding the full length hIL-1L1 protein was deposited at the American Type Culture Collection (1801 University Boulevard, Manassas, Va. 20110-2209; (703) 365-2700) on XXXX, XX, 1999 and has been assigned ATCC Designation No. XXXXXX. The hIL-1L1gene transcript includes a particularly long, approximately 2 kb 3′ untranslated region (UTR).

The murine homolog of hIL-1L1 has also been isolated and is herein referred to as mIL-1L1. The 1284 nucleotide mIL-1L1gene transcript is shown in FIG. 2 (SEQ ID No. 4) and includes 5′ and 3′ untranslated regions and a 465 nucleotide open reading frame encoding a 155 amino acid mIL-1L1polypeptide shown in FIG. 3B (SEQ ID No. 6). The mIL-1L1 polypeptide sequence is 90% identical to that of hIL-1L1 (FIG. 4), indicating that the encoded IL-1L1product has been highly conserved throughout evolution. A nucleic acid comprising the cDNA encoding the murine IL-1L1 polypeptide was deposited at the American Type Culture Collection (12301 Parklawn Drive, Rockville, Md.) on XXX XX, 1999 and has been assigned ATCC Designation No. XXXXXX. The mIL-1L1gene transcript includes an approximately 0.7 kb 3′ UTR. The mIL-1L1 3′ UTR shares limited homology with the 3′UTR of hIL-1L1. A 41 nucleotide conserved consensus sequence corresponding to 5′-ACAATNAAAANCCCNGATNCTGGTCTCTANTCNCATNAAAA-3′ (SEQ ID No. 12) is found beginning at nucleotide 1137 of SEQ ID No. 1 (hIL-1L1) and beginning at nucleotide 1146 of SEQ ID No. 4 (mIL-1L1).

An amino acid and nucleotide sequence analysis using the BLAST program (Altschul et al. (1990) J. Mol. Biol. 215:403) revealed that certain portions of the amino acid and nucleic acid sequences of the newly identified human and murine IL-1L1proteins and nucleic acids share certain sequence similarities with other IL-1 genes and encoded polypeptides. In particular, the IL-1L1polypeptides of the present invention include human and mouse IL-1L1polypeptide sequences characterized by one or more of: an IL1 domain, a casein kinase II phosphorylation site, a myristylation sequence, a transmembrane segment and IL-1 agonist and antagonist-homologous segements.

In one aspect, the invention features isolated IL-1L1 nucleic acid molecules. In one embodiment, the IL-1L1 nucleic acid is from a vertebrate. In a preferred embodiment, the IL-1L1 nucleic acid is from a mammal, e.g. a human. In an even more preferred embodiment, the nucleic acid has the nucleic acid sequence set forth in SEQ ID No. 1 or a portion thereof or comprises one of the the alternative 5′ ends specified in SEQ ID No. 2 or 3. In another embodiment of the invention, the nucleic acid is murine in origin and has the nucleic acid sequence set forth in SEQ ID No. 4 or a portion thereof. The disclosed molecules can be non-coding, (e.g. a probe, antisense, or ribozyme molecule) or can encode a functional IL-1L1 polypeptide (e.g. a polypeptide which specifically modulates biological activity by acting as either an agonist or antagonist of at least one bioactivity of the human IL-1L1polypeptide). In one embodiment, the nucleic acid molecule can hybridize to the IL-1L1 gene contained in ATCC designation number XXXXXX (hIL-1L1) or XXXXXX (mIL-1L1). In another embodiment, the nucleic acid of the present invention can hybridize to a vertebrate IL-1L1gene or to the complement of a vertebrate IL-1L1gene. In a further embodiment, the claimed nucleic acid can hybridize with a nucleic acid sequence shown in FIG. 1 (SEQ ID Nos. 1, 2 or 3) or a complement thereof. In another embodiment, the claimed nucleic acid can hybridize with a nucleic acid sequence shown in FIG. 2 (SEQ ID Nos. 4) or a complement thereof. In a preferred embodiment, the hybridization is conducted under mildly stringent or stringent conditions.

In further embodiments, the nucleic acid molecule is an IL-1L1 nucleic acid that is at least about 70%, preferably about 80%, more preferably about 85%, and even more preferably at least about 90% or 95% homologous to the nucleic acid shown as SEQ ID Nos. 1, 2, 3, or 4 or to the complement of the nucleic acid shown as SEQ ID Nos. 1, 2, 3, or 4. In a further embodiment, the nucleic acid molecule is an IL-1L1 nucleic acid that is at least about 70%, preferably at least about 80%, more preferably at least about 85% and even more preferably at least about 90% or 95% similar in sequence to the IL-1L1 nucleic acid contained in ATCC designation number XXXXXX or ATCC designation number XXXXXX.

The invention also provides probes and primers comprising substantially purified oligonucleotides, which correspond to a region of nucleotide sequence which hybridizes to at least about 6, at least about 10, at least about 15, at least about 20, or preferably at least about 25 consecutive nucleotides of the sequence set forth as SEQ ID Nos. 1, 2, or 3 or to SEQ ID No. 4 or their complements or, in preferred embodiments, to the sequences set forth in SEQ ID Nos. 10 or II or to naturally occurring mutants or allelic variants thereof. In preferred embodiments, the probe/primer further includes a label group attached thereto, which is capable of being detected.

For expression, the subject nucleic acids can be operably linked to a transcriptional regulatory sequence, e.g., at least one of a transcriptional promoter (e.g., for constitutive expression or inducible expression) or transcriptional enhancer sequence. The IL-1L1gene transcriptional regulatory sequences provide a unique means of regulating IL-1L1 polypeptides of the instant invention as well as for heterologous polypeptide encoding sequences. IL-1 locus encoded genes have been shown, for example, to respond to lipolypolysaccharide (LPS) induction with either activation (IL-1RAcP and IL-1RI) or repression (IL-1RII) (Penton-Rol (1999) J. Immunol. 162: 2931-8). Such regulatory sequences in conjunction with an IL-1L1 nucleic acid molecule can provide a useful vector for gene expression. This invention also describes host cells transfected with said expression vector whether prokaryotic or eukaryotic and in vitro (e.g. cell culture) and in vivo (e.g. transgenic) methods for producing IL-1L1 proteins by employing said expression vectors.

In another aspect, the invention features isolated IL-1L1polypeptides, preferably substantially pure preparations, e.g. of plasma purified or recombinantly produced polypeptides. The IL-1L1 polypeptide can comprise a full length protein or can comprise smaller fragments corresponding to one or more particular motifs/domains, or fragments comprising at least about 6, 10, 25, 50, 75, 100, 125, 130, 135, 140, 145, 150 or 155 amino acids in length. In particularly preferred embodiments, the subject polypeptide has an IL-1L1 bioactivity, for example, it is capable of binding to and/or otherwise altering the activity of a receptor, particularly an interleukin 1 receptor (IL-1R) family type or an interleukin 1 receptor accessory protein (IL-1RAcP) coreceptor.

In a one embodiment, the polypeptide is encoded by a nucleic acid which hybridizes with the nucleic acid sequence represented in SEQ ID Nos. 1, 2, 3, or 4. In a preferred embodiment, the polypeptide is encoded by a nucleic acid which hybridizes with a nucleic acid sequence from an IL-1L1 gene open reading frame (ORF) such as represented in SEQ ID No. 10 or 11. In a still more preferred embodiment, the IL-1L1polypeptide is comprised of the amino acid sequence set forth in SEQ ID No. 5 or SEQ ID No. 6. The subject IL-1L1 protein also includes within its scope modified proteins, e.g. proteins which are resistant to post-translational modification, for example, due to mutations which alter modification sites (such as tyrosine, threonine, serine or aspargine residues), or which prevent glycosylation of the protein, or which prevent interaction of the protein with intracellular proteins involved in signal transduction.

The IL-1L1 polypeptides of the present invention can be glycosylated, or conversely, by choice of the expression system or by modification of the protein sequence to preclude glycosylation, reduced carbohydrate analogs can also be provided. Glycosylated forms can be obtained based on derivatization with glycosaminoglycan chains. Also, IL-1L1 polypeptides can be generated which lack an endogenous signal sequence (though this is typically cleaved off even if present in the pro-form of the protein).

In yet another preferred embodiment, the invention features a purified or recombinant polypeptide, which has the ability to modulate, e.g., mimic or antagonize, an activity of a wild-type IL-1L1protein. Preferably, the polypeptide comprises an amino acid sequence identical or homologous to a sequence designated in SEQ ID No. 5 or SEQ ID No. 6.

Another aspect of the invention features chimeric molecules (e.g., fusion proteins) comprising an IL-1L1protein. For instance, the IL-1L1protein can be provided as a recombinant fusion protein which includes a second polypeptide portion, e.g., a second polypeptide having an amino acid sequence unrelated (heterologous) to the IL-1L1 polypeptide. A preferred IL-1L1 fusion protein is an immunoglobulin-IL-1L1fusion protein, in which an immunoglobulin constant region is fused to an IL-1L1 polypeptide.

Yet another aspect of the present invention concerns an immunogen comprising an IL-1L1polypeptide in an immunogenic preparation, the immunogen being capable of eliciting an immune response specific for an IL-1L1polypeptide; e.g. a humoral response, an antibody response and/or cellular response. In a preferred embodiment, the immunogen comprises an antigenic determinant, e.g. a unique determinant of a protein encoded by the nucleic acid set forth in SEQ ID No. 1, or 4; or a polypeptide sequence as set forth in SEQ ID Nos. 5 or 6.

A still further aspect of the present invention features antibodies and antibody preparations specifically reactive with an epitope of an IL-1L1 protein.

The invention also features transgenic non-human animals which include (and preferably express) a heterologous form of an IL-1L1gene described herein, or which misexpress an endogenous IL-1L1 gene (e.g., an animal in which expression of one or more of the subject IL-1L1 proteins is disrupted). Such transgenic animals can serve as animal models for studying cellular and/or tissue disorders comprising mutated or mis-expressed IL-1L1 alleles or for use in drug screening. Alternatively, such transgenic animals can be useful for expressing recombinant IL-1L1 polypeptides.

The invention further features assays and kits for determining whether an individual's IL-1L1 genes and/or proteins are defective or deficient (e.g in activity and/or level), and/or for determining the identity of IL-1L1 alleles. In one embodiment, the method comprises the step of determining the level of IL-1L1protein, the level of IL-1L1mRNA and/or the transcription rate of an IL-1L1 gene. In another preferred embodiment, the method comprises detecting, in a tissue of the subject, the presence or absence of a genetic alteration, which is characterized by at least one of the following: a deletion of one or more nucleotides from a gene; an addition of one or more nucleotides to the gene; a substitution of one or more nucleotides of the gene; a gross chromosomal rearrangement of the gene; an alteration in the level of a messenger RNA transcript of the gene; the presence of a non-wild type splicing pattern of a messenger RNA transcript of the gene; and/or a non-wild type level of the IL-1L1 protein.

For example, detecting a genetic alteration or the presence of a specific polymorphic region can include (i) providing a probe/primer comprised of an oligonucleotide which hybridizes to a sense or antisense sequence of an IL-1L1gene or naturally occurring mutants thereof, or 5′ or 3′ flanking sequences naturally associated with the IL-1L1 gene; (ii) contacting the probe/primer with an appropriate nucleic acid containing sample; and (iii) detecting, by hybridization of the probe/primer to the nucleic acid, the presence or absence of the genetic alteration. Particularly preferred embodiments comprise: 1) sequencing at least a portion of an IL-1L1 gene, 2) performing a single strand conformation polymorphism (SSCP) analysis to detect differences in electrophoretic mobility between mutant and wild-type nucleic acids; and 3) detecting or quantitating the level of an IL-1L1 protein in an immunoassay using an antibody which is specifically immunoreactive with a wild-type or mutated IL-1L1 protein.

Information obtained using the diagnostic assays described herein (alone or in conjunction with information on another genetic defect, which contributes to the same disease) is useful for diagnosing or confirming that a symptomatic subject has a genetic defect (e.g. in an IL-1L1gene or in a gene that regulates the expression of an IL-1L1gene), which causes or contributes to the particular disease or disorder. Alternatively, the information (alone or in conjunction with information on another genetic defect, which contributes to the same disease) can be used prognostically for predicting whether a non-symptomatic subject is likely to develop a disease or condition, which is caused by or contributed to by an abnormal IL-1L1 activity or protein level in a subject. In particular, the assays permit one to ascertain an individual's predilection to developing a condition, which is caused by or contributed to by a mutation in IL-1L1, where the mutation is a single nucleotide polymorphism (SNP). Based on the prognostic information, a doctor can recommend a regimen (e.g. diet or exercise) or therapeutic protocol useful for preventing or prolonging onset of the particular disease or condition in the individual.

In addition, knowledge of the particular alteration or alterations, resulting in defective or deficient IL-1L1genes or proteins in an individual, alone or in conjunction with information on other genetic defects contributing to the same disease (the genetic profile of the particular disease) allows customization of therapy for a particular disease to the individual's genetic profile, the goal of pharmacogenomics. For example, an individual's IL-1L1 genetic profile or the genetic profile of a disease or condition to which IL-1L1 genetic alterations cause or contribute, can enable a doctor to: 1) more effectively prescribe a drug that will address the molecular basis of the disease or condition; and 2) better determine the appropriate dosage of a particular drug. For example, the expression level of IL-1L1 proteins, alone or in conjunction with the expression level of other genes known to contribute to the same disease, can be measured in many patients at various stages of the disease to generate a transcriptional or expression profile of the disease. Expression patterns of individual patients can then be compared to the expression profile of the disease to determine the appropriate drug and dose to administer to the patient.

The ability to target populations expected to show the highest clinical benefit, based on the IL-1L1or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since the use of IL-1L1as a marker is useful for optimizing effective dose).

In another aspect, the invention provides methods for identifying a compound which modulates an IL-1L1 activity, e.g. the interaction between an IL-1L1 polypeptide and a target peptide In a preferred embodiment, the method includes the steps of (a) forming a reaction mixture including: (i) an IL-1L1 polypeptide, (ii) an IL-1L1binding partner (e.g., an IL-1L1 receptor or a heparin sulfate proteoglycan), and (iii) a test compound; and (b) detecting interaction of the IL-1L1 polypeptide and the IL-1L1 binding protein. A statistically significant change (potentiation or inhibition) in the interaction of the IL-1L1 polypeptide and IL-1L1binding protein in the presence of the test compound, relative to the interaction in the absence of the test compound, indicates a potential agonist (mimetic or potentiator) or antagonist (inhibitor) of IL-1L1bioactivity for the test compound. The reaction mixture can be a cell-free protein preparation, e.g., a reconstituted protein mixture or a cell lysate, or it can be a recombinant cell including a heterologous nucleic acid recombinantly expressing the IL-1L1 binding partner.

In preferred embodiments, the step of detecting interaction of the IL-1L1and IL-1L1binding partner is a competitive binding assay. In other preferred embodiments, at least one of the IL-1L1polypeptide and the IL-1L1binding partner comprises a detectable label, and interaction of the IL-1L1 and IL-1L1 binding partner is quantified by detecting the label in the complex. The detectable label can be, e.g., a radioisotope, a fluorescent compound, an enzyme, or an enzyme co-factor. In other embodiments, the complex is detected by an immunoassay.

Yet another exemplary embodiment provides an assay for screening test compounds to identify agents which modulate the amount of IL-1L1 produced by a cell. In one embodiment, the screening assay comprises contacting a cell transfected with a reporter gene operably linked to an IL-1L1 promoter with a test compound and determining the level of expression of the reporter gene. The reporter gene can encode, e.g., a gene product that gives rise to a detectable signal such as: color, fluorescence, luminescence, cell viability, relief of a cell nutritional requirement, cell growth, and drug resistance. For example, the reporter gene can encode a gene product selected from the group consisting of chloramphenicol acetyl transferase, luciferase, beta-galactosidase and alkaline phosphatase.

Also within the scope of the invention are methods for treating diseases or disorders which are associated with an aberrant IL-1L1 level or activity or which can benefit from modulation of the activity or level of IL-1L1. The methods comprise administering, e.g., either locally or systemically to a subject, a pharmaceutically effective amount of a composition comprising an IL-1L1therapeutic. Depending on the condition being treated, the therapeutic can be an IL-1L1 agonist or an IL-1L1 antagonist.

Other features and advantages of the invention will be apparent from the following detailed description and claims.

### 3. BRIEF DESCRIPTION OF THE FIGURES

FIG. 1 shows the nucleic acid sequence of two human IL-1L1cDNAs with alternative 5′ ends. The IL-1L1gene transcript includes a 2562 nucleotide common sequence (SEQ ID No. 1) which is 3′ polyadenylated and one of two different 5′ ends of 39 and 42 nucleotides (SEQ ID Nos. 2 and 3 respectively). The human IL-1L1open reading frame (ORF) (SEQ ID No. 10) is underlined.

FIG. 2 shows the nucleic acid sequence of a murine IL-1L1 cDNA (SEQ ID No. 4). The murine IL-1L1 ORF (SEQ ID No. 11) is underlined.

FIG. 3 shows the deduced polypeptide sequence encoded by human IL-1L1 (panel A; SEQ ID No. 5) and murine IL-1L1(panel B; SEQ ID No. 6).

FIG. 4 is a comparison of human and murine IL-1L1polypeptide sequences indicating conservative substitutions (+) and showing the consensus polypeptide sequence (SEQ ID No. 7).

FIG. 5 is a comparison of human IL-1L1polypeptide and human pro-IL-1ra polypeptide (SEQ ID No. 8) sequences indicating conservative substitutions (+) and showing the consensus polypeptide sequence (SEQ ID No. 9).

FIG. 6 is the polypeptide sequence of three recombinant forms of the IL-1L1 gene product which differ in their amino-terminal amino acid sequences.

FIG. 7 is a Northern blot showing tissue distribution of human IL-1L1 message.

FIG. 8 is a Zoo-Blot showing conservation of IL-1L1 gene sequences in mammals.

FIG. 9 shows the chromosomal position of the IL-1L1gene within the IL-1 locus of human chromosome 2 as well as the intron/exon structure of the IL-1L1gene.

FIG. 10 illustrates certain details of the splicing of IL-1L1transcripts.

FIG. 11 depicts the complete genomic sequence of the IL1L1 gene.

FIG. 12 shows the immumprecipitation of native IL-1L1 protein from JEG-3 cells.

FIG. 13 depicts the alignment of human and mouse IL1L1 genes with other members of the IL-1-like gene family in humans. Conserved residues are boxed and the beta-sheet secondary structure of the folded protein is indicated.

### 4. DETAILED DESCRIPTION OF THE INVENTION

4.1. General

The invention is based at least in part on the discovery of a gene within the IL-1 gene cluster locus that encodes a protein which is homologous to the IL-1 receptor antagonist (IL-1ra) and IL-1b polypeptides. The IL-1L1protein is thus related to both a pro-inflammatory cytokine (IL-1b) as well as an inflammatory cytokine antagonist (IL-1ra). The IL-1L1 gene name is derived from the fact that it is located in the Interval between the IL-1Beta encoding gene and the IL-1Receptor antagonist encoding gene. Thus, the genes and proteins disclosed herein are referred to as IL-1L1 genes and IL-1L1proteins.

The IL-1L1gene was isolated by utilizing the the 680 kb CEPH yeast artificial chromosome (YAC) clone 766E12, which contains the IL-1 cluster (Nothwang et al. (1997). YAC clone 766E12 was isolated from a yeast strain bearing the clone by pulsed field electrophoresis and the resulting DNA was used as a driver in the selection of IL-1 encoded messages (Morgan et al. (1992) Nucleic Acids Res. 20: 5173-79). cDNA was derived from human peripheral blood mononuclear cells which were cultured for 6 hours in the presence of 10 ng/ml phorbol myristate acetate and 100 ng/ml E. coli lipopolysaccharide.

The selected cDNAs were amplified and the amplified cDNA fragments were cloned into pNEB193 after two rounds of affinity selection and further amplification. A random sample of one hundred of the resulting clones were sequenced in their entirety. The IL-1L1expressed sequence tag (EST) corresponded to one of these overlapping sequences, derived from three overlapping cDNA fragments which together comprise an approximately 530 bp contiguous sequence. This sequence was found to be homologous to both a 374 bp human placenta-derived cDNA (GenBank Accession No. R70041) and a 348 bp human placental cDNA (GenBank Accession No. R70089). On testing, it was found to hybridize specifically with P1 artificial chromosome 131J6 from the Gingrich human PAC library, which maps between the IL1B and IL1RN genes adjacent to ICRF 700G1305 from the ICRF human P1 Library (Nothwang et al. (1996) Genomics 41: 370-8). This latter clone contains the IL-1ra gene (IL1RN). No open reading frame was present either in the database EST sequences nor in these novel EST sequences, and subsequent analysis revealed that these sequences are contained within the 3′ untranslated (UTR) region of the gene message.

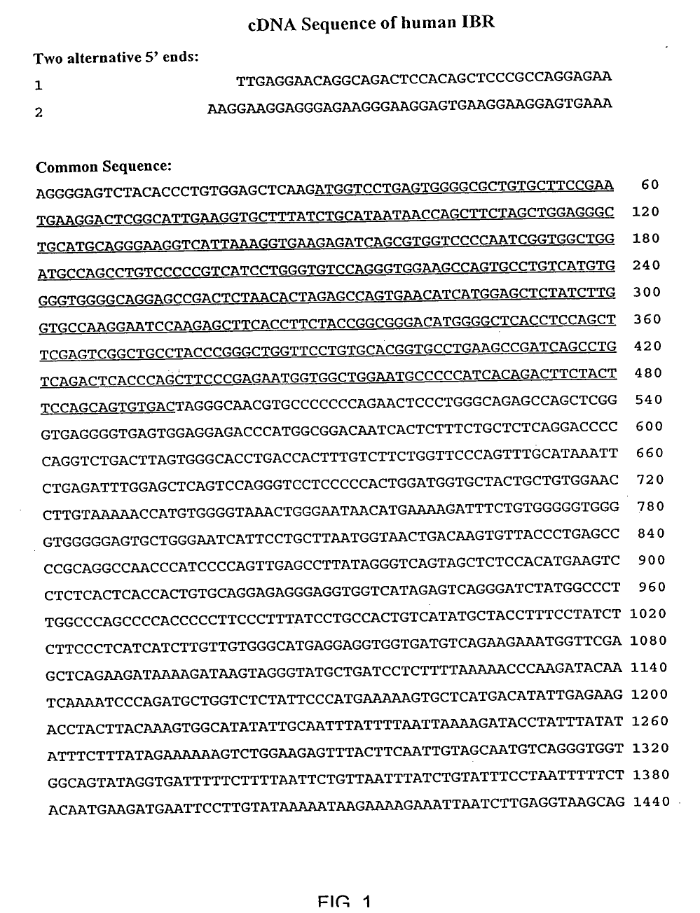

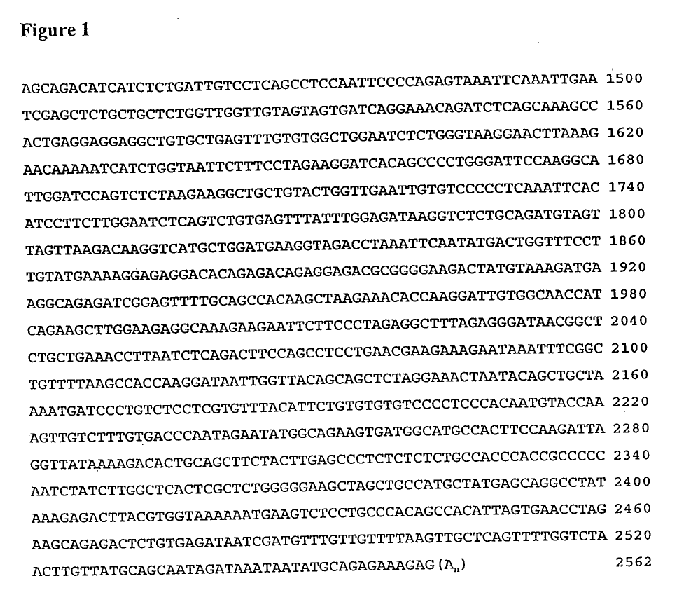

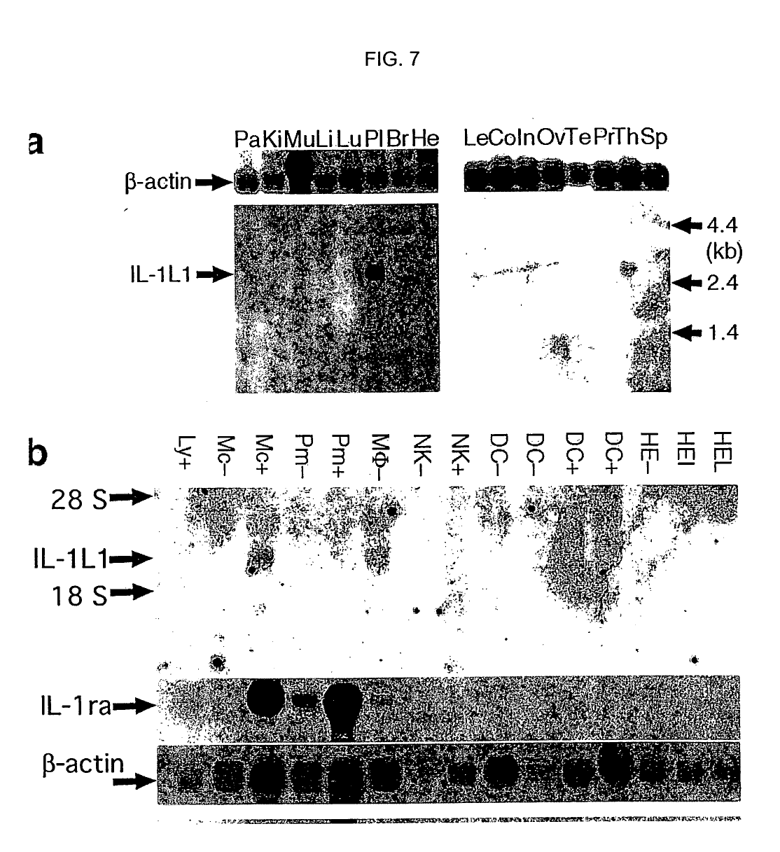

Northern blot analysis using this 3′ fragment as a probe revealed an approximately 2.6 kb transcript present in human placenta (FIG. 7). The full-length cDNA was obtained by first using the 3′ fragment as a probe to isolate a 1.6 kb IL-1L1 cDNA fragment from the H9 placenta cDNA plasmid library obtained from the HGMP (Human Genome Mapping Project). This 1.6 kb clone was polyadenylated but did not contain an apparent open reading frame. The 5′ end of this clone was then used to design primers for the amplification of the 5′ end of the full-length IL-1L1transcript from human placenta-derived cDNA. The resulting product was approximately 1.1 kb in length and overlapped the sequence of the 1.6kb clone. Subsequent analysis has revealed that the IL-1L1gene encodes two alternative transcripts which differ slightly in their 5′ non-coding regions (FIG. 1). The assembled full-length transcripts are approximately 2.6 kb, consistent with the placenta mRNA detected by Northern analysis. A similar sized transcript was also detected in human thymus with a probe containing the IL-1L1coding sequence (FIG. 7). The complete human cDNA was found to be homologous to the following EST cDNA fragments in the GenBank database: GenBank Accession No. A1040890 (human parathyroid tumor-derived EST of 485 bp homologous to the 3′ UTR of the IL-1L1antisense strand extending from nucleotide 955 to nucleotide 1278 of the common human transcript sequence shown in FIG. 1); GenBank Accession No. A1469873 (a human 442 bp EST homologous to the IL-1L1 3′ UTR antisense strand extending from nucleotide 2133 to nucleotide 2561); GenBank Accession No. AA722902 (a human fetal heart-derived 414 bp EST homologous to the IL-1L1 3′ UTR antisense strand extending from nucleotide 2141 to nucleotide 2555); GenBank Accession No. A167887 (a human parathyroid-derived 410 bp EST homologous to the IL-1L1 3′ UTR antisense strand extending from nucleotide 871 to nucleotide 1278); GenBank Accession No. R70041 (a human placenta-derived 317 bp EST homologous to the IL-1L1 3′ UTR antisense strand extending from nucleotide 2191 to nucleotide 2552); GenBank Accession No. R70089 (a human placenta-derived 348 bp EST homologous to the IL-1L1 3′ UTR sense strand extending from nucleotide 1828 to nucleotide 2144); and to GenBank Accession No. W08205 (a murine-derived 382 bp EST homologous to the IL-1L1 5′ leader and coding sequence sense strand extending from nucleotide 5 to nucleotide 308).

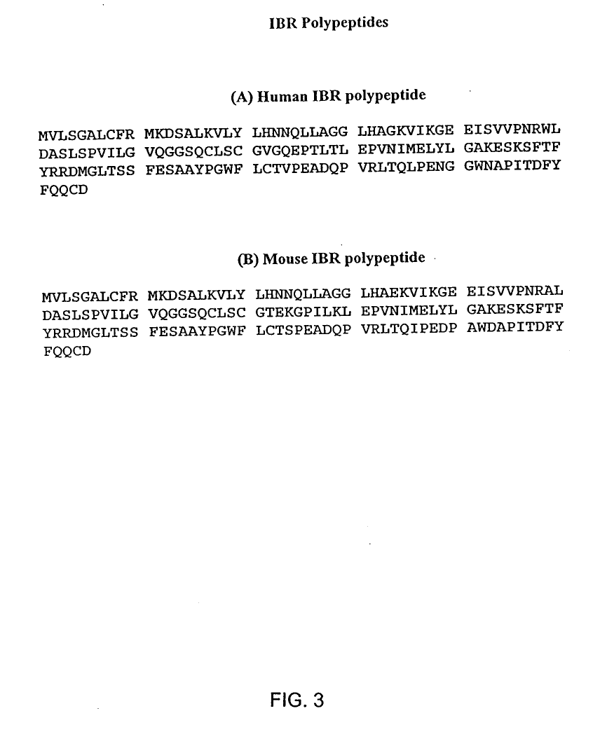

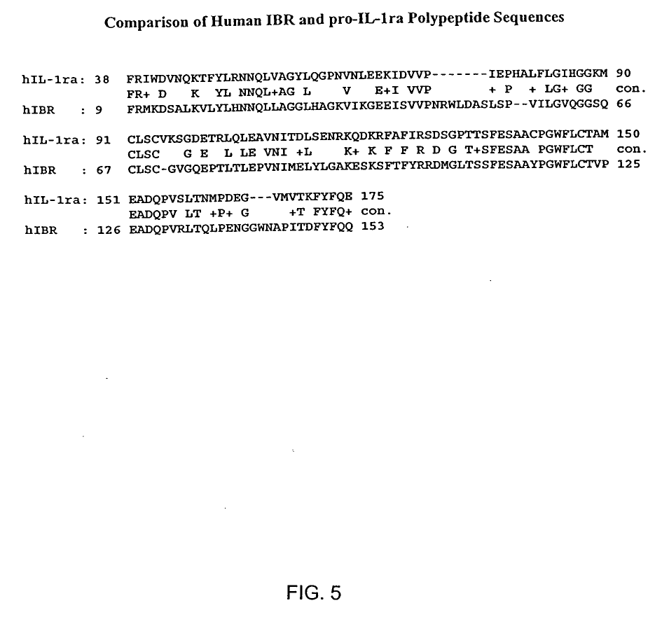

The human IL-1L1transcript encodes a 155 amino acid polypeptide (FIG. 3, panel A) and the coding sequence (underlined in FIG. 1) occupies less than the first 600 nucleotides of the transcript, leaving an extensive 3′ untranslated region of approximately 2 kb. The human IL-1L1 polypeptide sequence is homologous to human IL-1ra (SwissProt (SP) Accession No. P18510, 73 identities/155 amino acids or 47% identity overall see FIG. 5) as well as murine (SP No. P25085), rat (SP No. P25086) and rabbit (SP No. P26890) homologs of the IL-1ra encoding gene. The IL-1L1 polypeptide is also homologous to human IL-1 beta polypeptide (SP No. P01584 42 identifies/155 amino acids or 27% identity overall) as well as red deer (SP No. P51754), sheep (SP No. 21621), bovine (SP No. P09428), murine (SP No. P10749), rat (SP No. Q63264) and other IL-1 beta protein homologs.

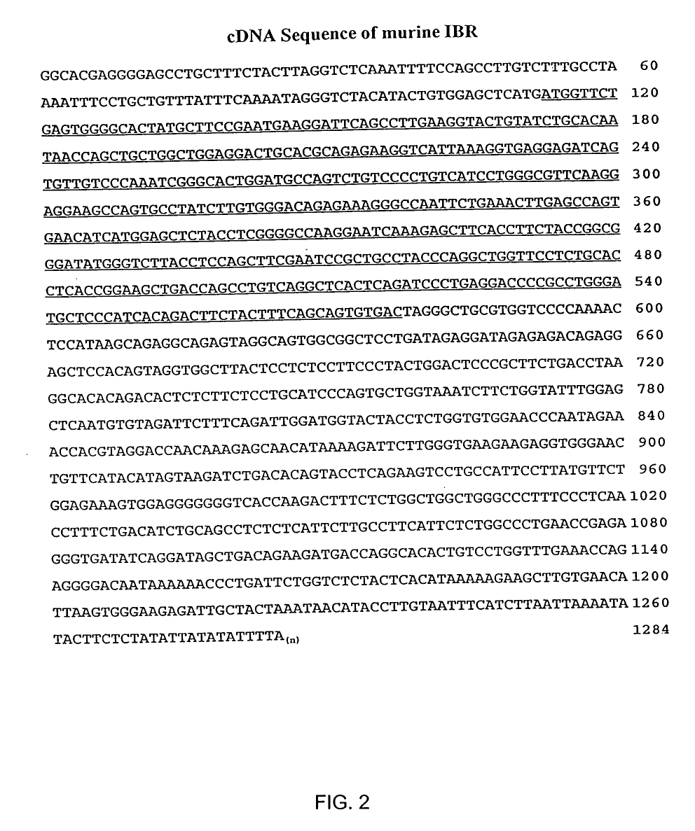

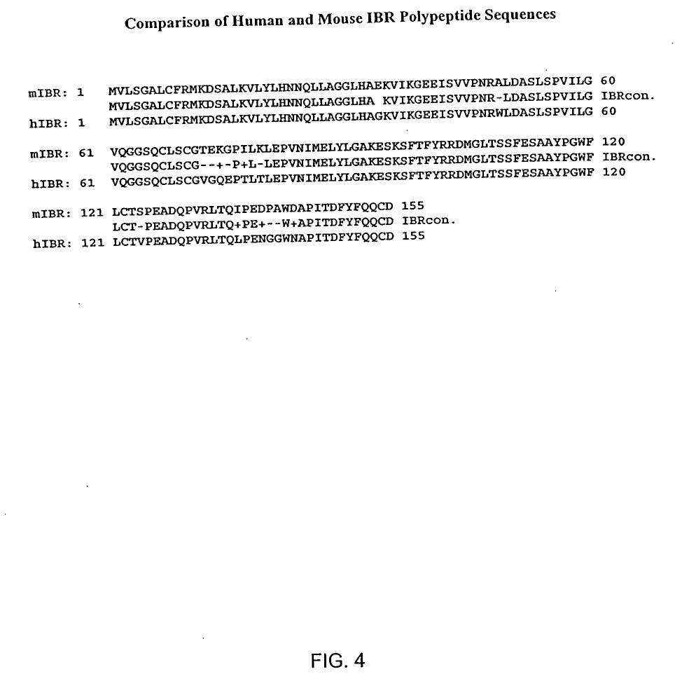

A 1.2 kb murine IL-1L1 cDNA was also obtained and sequenced (see FIG. 2). This message is predicted to encode a 155 amino acid polypeptide (FIG. 3, panel B) which is 90% identical (141 identities over 155 amino acid sequence) to that of human IL-1L1 as shown in FIG. 4. The nucleic acid sequence of the coding region of the 1.2 kb murine IL-1L1cDNA shares extensive homology with the sequence of the coding region of the 2.6 kb human IL-1L1 cDNA (84% identical to the human IL-1L1 sequence spanning nucleotides 7 to 500). The 1.2 kb murine IL-1L1 clone is polyadenylated and shares a short region of homology in its 3′ untranslated region with the human IL-1L1 3′ UTR sequence. This region of 83% sequence identity extends from nucleotide 1137 to nucleotide 1177 of the common human IL-1L1 transcript sequence and from nucleotide 1146 to nucleotide 1186 of the murine IL-1L1 sequence. A 41 bp conserved consensus sequence:

5′-ACAATNAAAANCCCNGATNCTGGTCTCTANTCNCATNAAAA-3′ (SEQ ID No. 12) found in both murine and human IL-1L1 3′UTRs contains a number of nucleic acid sequence motifs including: GATA-1 and GATA-2 binding sites (Merika and Orkin (1993) Mol.Cell.Biol. 13: 3999-4010); an Ikaros binding site (Molnar and Georgopoulos (1994) Mol. Cell. Biol. 14:8292-8303); and a retroviral polyA downstream element. Such conserved downstream nucleotide sequences may function to control: RNA polymerase initiation and/or termination, mRNA polyadenylation, mRNA stability, or IL-1L1 translation initiation or termination.

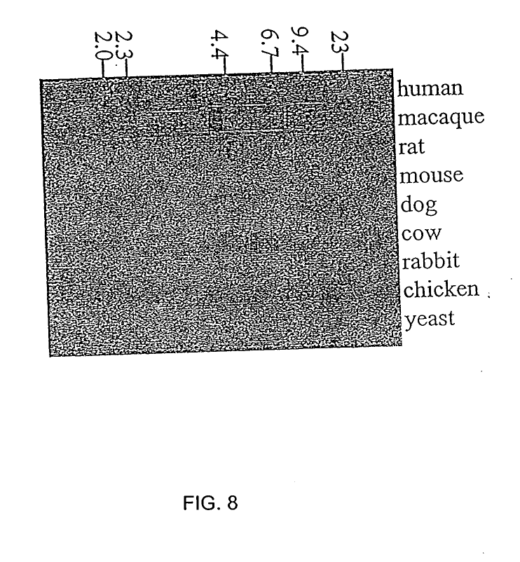

A low stringency Southern blot hybridization was performed using a human IL-1L1probe on a Southern transfer containing genomic DNA digests from human, macaque, rat, mouse, dog, cow, rabbit, chicken and yeast. This Zoo-blot hybridization revealed a single homologous band in all mammals tested (FIG. 8). Therefore the IL-1L1gene appears to have been conserved throughout mammalian evolution.

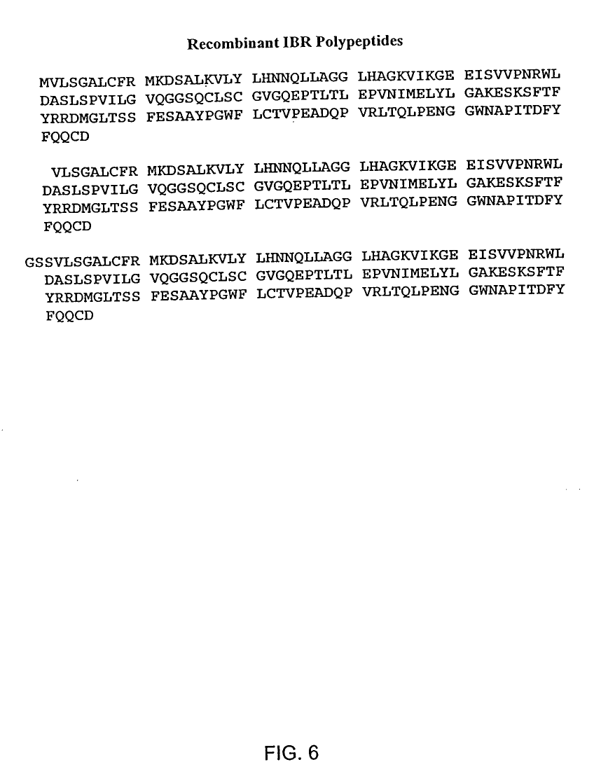

Recombinant human IL-1 receptor antagonist and IL-1L1 polypeptides have been prepared and purified by nickel chelate chromatography followed by elution with imidazole and processing with thrombin to yield the polypeptide sequences shown in FIG. 6. Nuclear magnetic resonance studies with N¹⁵ and C¹³ double-labeled IL-1L1 protein has indicated that the IL-1L1protein is folded largely as a beta-sheet structure which is consistent with the structure of human IL-1ra.

The recombinant IL-1ra has also been used to raise polyclonal antibodies in rabbits. The choriocarcinoma cell line JEG-3 has been shown to produce the IL-1L1mRNA constitutively. Metabolically labeled protein from this cell line was immunoprecipitated with the anti-IL-1L1 rabbit polyclonal antisera. The results indicate that a 17 kDa sized protein, consistent with a predicted molecular weight of IL-1L1, was immunoprecipitated.

4.2 Definitions

For convenience, the meaning of certain terms and phrases employed in the specification, examples, and appended claims are provided below.

The term “aberrant activity”, as applied to an activity of a polypeptide such as IL-1L1, refers to an activity which differs from the activity of the wild-type or native polypeptide or which differs from the activity of the polypeptide in a healthy subject. An activity of a polypeptide can be aberrant because it is stronger than the activity of its native counterpart. Alternatively, an activity can be aberrant because it is weaker or absent relative to the activity of its native counterpart. An aberrant activity can also be a change in an activity. For example an aberrant polypeptide can interact with a different target peptide. A cell can have an aberrant IL-1L1 activity due to overexpression or underexpression of the gene encoding IL-1L1.

The term “agonist”, as used herein, is meant to refer to an agent that mimics or upregulates (e.g. potentiates or supplements) an IL-1L1 bioactivity. An IL-1L1 agonist can be a wild-type IL-1L1 protein or derivative thereof having at least one bioactivity of the wild-type IL-1L1, e.g. IL-1B receptor binding activity. An IL-1L1 therapeutic can also be a compound that upregulates expression of an IL-1L1gene or which increases at least one bioactivity of an IL-1L1 protein. An agonist can also be a compound which increases the interaction of an IL-1L1 polypeptide with another molecule, e.g, an IL-1 type I or type II receptor. Such an agonist may, for example, increase the binding of IL-1L1 to an IL-1 type I receptor, thereby promoting an IL-1L1-dependent blocking of the inflammatory signaling of IL-1a and IL-1b. Alternatively, an IL-1L1agonist may increase the binding of IL-1L1to an IL-1 type II or “decoy receptor.” The type II IL-1 receptor is present on B cells and acts to attenuate IL-1a/IL-1b dependent inflammatory signaling. Accordingly an IL-1L1agonist may indirectly promote the inflammatory signaling of IL-1a and IL-1b.

The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. Frequently occurring sequence variations include transition mutations (i.e. purine to purine substitutions and pyrimidine to pyrimidine substitutions, e.g. A to G or C to T), transversion mutations (i.e. purine to pyrimidine and pyrimidine to purine substitutions, e.g. A to T or C to G), and alteration in repetitive DNA sequences (e.g. expansions and contractions of trinucleotide repeat and other tandem repeat sequences). An allele of a gene can also be a form of a gene containing a mutation. The term “allelic variant of a polymorphic region of an IL-1L1gene” refers to a region of an IL-1L1gene having one or several nucleotide sequences found in that region of the gene in other individuals.

“Antagonist” as used herein is meant to refer to an agent that downregulates (e.g. suppresses or inhibits) at least one IL-1L1 bioactivity. An IL-1L1 antagonist can be a compound which inhibits or decreases the interaction between an IL-1L1 protein and another molecule, e.g., a receptor, such as an IL-1 receptor (IL-1 R). Accordingly, a preferred antagonist is a compound which inhibits or decreases binding to an IL-1B receptor and thereby blocks subsequent activation of the IL-1B receptor. An antagonist can also be a compound that downregulates expression of an IL-1L1 gene or which reduces the amount of IL-1L1protein present. The IL-1L1antagonist can be a dominant negative form of an IL-1L1 polypeptide, e.g., a form of an IL-1L1polypeptide which is capable of interacting with a target peptide, e.g., an IL-1 type I receptor, but which is not capable of receptor activation. The IL-1L1antagonist can also be a nucleic acid encoding a dominant negative form of an IL-1L1polypeptide, an IL-1L1antisense nucleic acid, or a ribozyme capable of interacting specifically with an IL-1L1 RNA. Yet other IL-1L1 antagonists are molecules which bind to an IL-1L1 polypeptide and inhibit its action. Such molecules include peptides, e.g., forms of IL-1L1 target peptides which do not have biological activity, and which inhibit binding to IL-1L1 target molecules, such as an IL-1L1 receptor, preferably an IL-1 type I receptor. Alternatively, an IL-1L1 antagonist may act to inhibit an IL-1 type II receptor. Type II IL-1 receptors are so-called “decoy” receptors present on B cells which bind to IL-1a and IL-1b and prevent their interaction with IL-1 type I receptors—thereby antagonizing IL-1 type 1 receptor mediated activities. Certain IL-1L1 polypeptides may bind to and antagonize the IL-1 type II decoy receptor and thereby promote the inflammatory signaling of IL-1a and IL-1b. Accordingly, certain IL-1L1antagonists may act to prevent this from occurring, thereby attenuating the inflammatory signaling of IL-1a and IL-1b. Such IL-1L1antagonist polypeptides bind the active site of IL-1L1 and prevent it from interacting with target peptides, e.g., IL-1 type I and type II receptors. In yet another preferred embodiment, the IL-1L1 antagonist is a small molecule, such as a molecule capable of inhibiting the interaction between an IL-1L1polypeptide and a target IL-1L1R. Alternatively, the small molecule can be antagonized by interacting with sites other than the IL-1L1R binding site, such as a heparin sulfate proteoglycan binding site.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

A disease, disorder, or condition “associated with” or “characterized by” an aberrant expression of a nucleic acid refers to a disease, disorder, or condition in a subject which is caused by, contributed to by, or causative of an aberrant level of expression of a nucleic acid.

As used herein the term “bioactive fragment of an IL-1L1polypeptide” refers to a fragment of a full-length IL-1L1polypeptide, wherein the fragment specifically mimics or antagonizes the activity of a wild-type IL-1L1 polypeptide. The bioactive fragment preferably is a fragment capable of interacting with an interleukin type I or type II receptor.

“Biological activity” or “bioactivity” or “activity” or “biological function”, which are used interchangeably, for the purposes herein means an effector or antigenic function that is directly or indirectly performed by an IL-1L1 polypeptide (whether in its native or denatured conformation), or by any subsequence thereof. Biological activities include binding to a target peptide, e.g., an IL-1 receptor (IL-1R), preferably an IL-1 type I receptor. An IL-1L1bioactivity can be modulated by directly affecting an IL-1L1 polypeptide. Alternatively, an IL-1L1 bioactivity can be modulated by modulating the level of an IL-1L1polypeptide, such as by modulating expression of an IL-1L1gene.

The term “biomarker” refers a biological molecule, e.g., a nucleic acid, peptide, hormone, etc., whose presence or concentration can be detected and correlated with a known condition, such as a disease state.

“Cells”, “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric polypeptide” or “fusion polypeptide” is a fusion of a first amino acid sequence encoding one of the subject IL-1L1 polypeptides with a second amino acid sequence defining a domain (e.g. polypeptide portion) foreign to and not substantially homologous with any domain of an IL-1L1polypeptide. A chimeric polypeptide may present a foreign domain which is found (albeit in a different polypeptide) in an organism which also expresses the first polypeptide, or it may be an “interspecies”, “intergenic”, etc. fusion of polypeptide structures expressed by different kinds of organisms. In general, a fusion polypeptide can be represented by the general formula X-IL-1L1-Y, wherein IL-1L1 represents a portion of the polypeptide which is derived from an IL-1L1polypeptide, and X and Y are independently absent or represent amino acid sequences which are not related to an IL-1L1sequence in an organism, including naturally occurring mutants.

A “delivery complex” shall mean a targeting means (e.g. a molecule that results in higher affinity binding of a gene, protein, polypeptide or peptide to a target cell surface and/or increased cellular or nuclear uptake by a target cell). Examples of targeting means include: sterols (e.g. cholesterol), lipids (e.g. a cationic lipid, virosome or liposome), viruses (e.g. adenovirus, adeno-associated virus, and retrovirus) or target cell specific binding agents (e.g. ligands recognized by target cell specific receptors). Preferred complexes are sufficiently stable in vivo to prevent significant uncoupling prior to internalization by the target cell. However, the complex is cleavable under appropriate conditions within the cell so that the gene, protein, polypeptide or peptide is released in a functional form.

As is well known, genes may exist in single or multiple copies within the genome of an individual. Such duplicate genes may be identical or may have certain modifications, including nucleotide substitutions, additions or deletions, which all still code for polypeptides having substantially the same activity. The term “DNA sequence encoding an IL-1L1polypeptide” may thus refer to one or more genes within a particular individual. Moreover, certain differences in nucleotide sequences may exist between individual organisms, which are called alleles. Such allelic differences may or may not result in differences in amino acid sequence of the encoded polypeptide yet still encode a polypeptide with the same biological activity.

The term “equivalent” is understood to include nucleotide sequences encoding functionally equivalent polypeptides. Equivalent nucleotide sequences will include sequences that differ by one or more nucleotide substitutions, additions or deletions, such as allelic variants; and will, therefore, include sequences that differ from the nucleotide sequence of the nucleic acids shown in, for example, SEQ ID Nos. 1 and 4, due to the degeneracy of the genetic code.

The term “haplotype” as used herein is intended to refer to a set of alleles that are inherited together as a group (are in linkage disequilIL-1L1ium) at statistically significant levels (p_{corr}<0.05). As used herein, the phrase “an IL-1 haplotype” refers to a haplotype in the IL-1 loci which may include polymorphic variations of IL-1L1 gene sequences.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the IL-1L1sequences of the present invention.

As used herein, the term “IL-1L1” refers to the interleukin 1-like gene 1, described herein, which encodes the IL-1L1 protein. As used herein, the term IL-1L1 is used interchangeably with the term “IBR” gene or protein, a second name for IL-1L1 based upon its location within the IL-1 locus (the IL-1L1 gene is located in the Interval between the IL-1Beta and the IL-1 Receptor Antagonist encoding genes). Further as used herein, the IL-1L1 gene is also referred to as the IL1L1 gene.

The term “IL-1L1nucleic acid” refers to a nucleic acid encoding an IL-1L1 protein, such as nucleic acids having SEQ ID No. 10 or 11 or fragments thereof, a complement thereof, and derivatives thereof.

The terms “IL-1L1polypeptide” and “IL-1L1protein” are intended to encompass polypeptides comprising the amino acid sequence shown as SEQ ID No. 5 or SEQ ID No. 6 or fragments thereof, and homologs thereof and include agonist and antagonist polypeptides.

The term “IL-1L1receptor” or “IL-1L1R” refers to various cell membrane bound protein receptors capable of binding to and/or transducing a signal from IL-1L1 such as, for example, the IL-1 type I and type II receptors. The term “IL-1 receptor” or “IL-1R” refers to various cell membrane bound protein receptors capable of binding to and/or transducing a signal from any or all of the IL-1 related ligands—e.g. IL-1a, IL-1b, and IL-1ra (receptor antagonist). Such receptors include the IL-1 type I receptor, present on T cells, fibroblasts, and connective tissue, and the IL-1 type II receptor, present on B cells. As used herein, the term IL-1L1R further refers to the IL-1R accessory protein (or IL1R AcP), which is related by homology to the Ig superfamily within its extracellular domain, and also related by limited homology to both type I and type II IL-1 receptors. The IL-1R AcP appears to form a complex with the type I IL-1 receptor, thereby transducing the pro-inflammatory signal of either IL-1 alpha or IL-1 beta. As used herein, the term IL-1L1 receptor (or IL-1L1R) further refers to IL-1 receptor related genes such as the orphan receptors ST2/T1 and IL-1rrp2. Other IL-1 receptor-related genes within the scope of the term IL-1L1R, as used herein, include MyD88 and rsc786.

The term “IL-1L1 therapeutic” refers to various forms of IL-1L1 polypeptides, as well as peptidomimetics, nucleic acids, or small molecules, which can modulate at least one activity of an IL-1L1 polypeptide, e.g., interaction with an IL-1L1receptor interaction with and/or an IL-1L1coreceptor, by mimicking or potentiating (agonizing) or inhibiting (antagonizing) the effects of a naturally-occurring IL-1L1 polypeptide. An IL-1L1 therapeutic which mimics or potentiates the activity of a wild-type IL-1L1polypeptide is an “IL-1L1agonist”. Conversely, an IL-1L1therapeutic which inhibits the activity of a wild-type IL-1L1 polypeptide is an “IL-1L1 antagonist”.

The terms “IL-1 gene cluster” and “IL-1 loci” as used herein include all the nucleic acid at or near the 2q13 region of chromosome 2, including at least the IL-1L1, IL-1A, IL-1B and IL-1RN genes and any other linked sequences. (Nicklin et al., Genomics 19: 382-84, 1994). The terms “IL-1A”, “IL-1B”, and “IL-1RN” as used herein refer to the genes coding for IL-1, IL-1, and IL-1 receptor antagonist, respectively. The gene accession number for IL-1A, IL-1B, and IL-1RN are X03833, X04500, and X64532, respectively.

“IL-1 functional mutation” refers to a mutation within the IL-1 gene cluster that results in an altered phenotype (i.e. effects the function of an IL-1 gene or protein). Examples include: IL-1A(+4845) allele 2, IL-1B (+3954) allele 2, IL-1B (+6912) allele 2 and IL-1RN (+2018) allele 2.

“IL-1X (Z) allele Y” refers to a particular allelic form, designated Y, occurring at an IL-1 locus polymorphic site in gene X, wherein X is IL-1A, B, or RN or some other gene in the IL-1 gene loci, and positioned at or near nucleotide Z, wherein nucleotide Z is numbered relative to the major transcriptional start site, which is nucleotide +1, of the particular IL-1 gene X. As further used herein, the term “IL-1X allele (Z)” refers to all alleles of an IL-1 polymorphic site in gene X positioned at or near nucleotide Z. For example, the term “IL-1RN (+2018) allele” refers to alternative forms of the IL-1RN gene at marker +2018. “IL-1RN (+2018) allele 1” refers to a form of the IL-1RN gene which contains a cytosine (C) at position +2018 of the sense strand. Clay et al., Hum. Genet. 97:723-26, 1996. “IL-1RN (+2018) allele 2” refers to a form of the IL-1RN gene which contains a thymine (T) at position +2018 of the plus strand. When a subject has two identical IL-1RN alleles, the subject is said to be homozygous, or to have the homozygous state. When a subject has two different IL-1RN alleles, the subject is said to be heterozygous, or to have the heterozygous state. The term “IL-1RN (+2018) allele 2,2” refers to the homozygous IL-1 RN (+2018) allele 2 state. Conversely, the term “IL-1RN (+2018) allele 1,1” refers to the homozygous IL-1 RN (+2018) allele I state. The term “IL-1RN (+2018) allele 1,2” refers to the heterozygous allele 1 and 2 state.

“IL-1 related” as used herein is meant to include all genes related to the human IL-1 locus genes on human chromosome 2 (2q 12-14). These include IL-1 genes of the human IL-1 gene cluster located at chromosome 2 (2q 13-14) which include: the IL-1L1 gene, the IL-1A gene which encodes interleukin-1α, the IL-1B gene which encodes interleukin-1β, and the IL-1RN (or IL-1ra) gene which encodes the interleukin-1 receptor antagonist. Furthermore these IL-1 related genes include the type I and type II human IL-1 receptor genes located on human chromosome 2 (2q12) and their mouse homologs located on mouse chromosome 1 at position 19.5 cM. Interleukin-1α, interleukin-1β, and interleukin-1RN are related in so much as they all bind to IL-1 type I receptors, however only interleukin-1α and interleukin-1β are agonist ligands which activate IL-1 type I receptors, while interleukin-1RN is a naturally occurring antagonist ligand. Where the term “IL-1” is used in reference to a gene product or polypeptide, it is meant to refer to all gene products encoded by the interleukin-1 locus on human chromosome 2 (2q 12-14) and their corresponding homologs from other species or functional variants thereof. The term IL-1 thus includes secreted polypeptides which promote an inflammatory response, such as IL-1α and IL-1β, as well as a secreted polypeptide which antagonize inflammatory responses, such as IL-1 receptor antagonist and the IL-1 type II (decoy) receptor.

An “IL-1 receptor” or “IL-1R” refers to various cell membrane bound protein receptors capable of binding to and/or transducing a signal from IL-1 locus-encoded ligand such as IL-1a, IL-1b, IL-1ra or IL-1L1. The term applies to any of the proteins which are capable of binding interleukin-1 (IL-1) molecules and, in their native configuration as mammalian plasma membrane proteins, presumably play a role in transducing the signal provided by IL-1 to a cell. As used herein, the term includes analogs of native proteins with IL-1-binding or signal transducing activity. Examples include the human and murine IL-1 receptors such as the 80 kDa polypeptide designated IL-1 receptor type I (IL-1R type I; Sims et al. (1988) Science 241: 585-9) which is expressed on T cells, fibroblasts, and epithelial cells and the the 68 kDa polypeptide designated IL-1 receptor type II (IL-1R type II; Bomsztyk et al. (1989) Proc. Natl. Acad. Sci. U.S.A. 86: 8034-8) which is expressed on B cells, neutrophils, and macrophages, both of which are structurally related to the immunoglobuling(Ig) superfamily of proteins. IL-1 receptors of the invention include those described in described in U.S. Pat. Nos. 4,968,607, 5,081,228, and 5,350,683. The term “IL-1 nucleic acid” refers to a nucleic acid encoding an IL-1 protein.

An “IL-1 polypeptide” and “IL-1 protein” are intended to encompass polypeptides comprising the amino acid sequence encoded by the IL-1 genomic DNA, or fragments thereof, and homologs thereof and include agonist and antagonist polypeptides. These IL-1 proteins include IL-1L1, IL-1a, IL-1b and the IL-1ra polypeptides.

“Increased risk” refers to a statistically higher frequency of occurrence of the disease or condition in an individual carrying a particular polymorphic allele in comparison to the frequency of occurrence of the disease or condition in a member of a population that does not carry the particular polymorphic allele.

The term “interact” as used herein is meant to include detectable relationships or association (e.g. biochemical interactions) between molecules, such as interaction between protein-protein, protein-nucleic acid, nucleic acid-nucleic acid, and protein-small molecule or nucleic acid-small molecule in nature.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject IL-1L1 polypeptides preferably includes no more than 10 kilobases (kb) of nucleic acid sequence which naturally immediately flanks the IL-1L1gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

A “knock-in” transgenic animal refers to an animal that has had a modified gene introduced into its genome and the modified gene can be of exogenous or endogenous origin.

A “knock-out” transgenic animal refers to an animal in which there is partial or complete suppression of the expression of an endogenous gene (e.g, based on deletion of at least a portion of the gene, replacement of at least a portion of the gene with a second sequence, introduction of stop codons, the mutation of bases encoding critical amino acids, or the removal of an intron junction, etc.). In preferred embodimbents, the “knock-out” gene locus corresponding to the modified endogenous gene no longer encodes a functional polypeptide activity and is said to be a “null” allele. Accordingly, knock-out transgenic animals of the present invention include those carrying one IL-1 gene null mutation, such as IL-1L1null allele heterozygous animals, and those carrying two IL-1 gene null muations, such as IL-1L1 null allele homozygous animals.

A “knock-out construct” refers to a nucleic acid sequence that can be used to decrease or suppress expression of a protein encoded by endogenous DNA sequences in a cell. In a simple example, the knock-out construct is comprised of a gene, such as the IL-1RN gene, with a deletion in a critical portion of the gene so that active protein cannot be expressed therefrom. Alternatively, a number of termination codons can be added to the native gene to cause early termination of the protein or an intron junction can be inactivated. In a typical knock-out construct, some portion of the gene is replaced with a selectable marker (such as the neo gene) so that the gene can be represented as follows: IL-1RN 5′/neo/ IL-1RN 3′, where IL-1RN5′ and IL-1RN 3′, refer to genomic or cDNA sequences which are, respectively, upstream and downstream relative to a portion of the IL-1RN gene and where neo refers to a neomycin resistance gene. In another knock-out construct, a second selectable marker is added in a flanking position so that the gene can be represented as: IL-1RN/neo/IL-1RN/TK, where TK is a thymidine kinase gene which can be added to either the IL-1RN5′ or the IL-1RN3′ sequence of the preceding construct and which further can be selected against (i.e. is a negative selectable marker) in appropriate media. This two-marker construct allows the selection of homologous recombination events, which removes the flanking TK marker, from non-homologous recombination events which typically retain the TK sequences. The gene deletion and/or replacement can be from the exons, introns, especially intron junctions, and/or the regulatory regions such as promoters.

“Linkage disequilibrium” refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at expected frequencies are said to be in “linkage equilibrium”. The cause of linkage disequilibrium is often unclear. It can be due to selection for certain allele combinations or to recent admixture of genetically heterogeneous populations. In addition, in the case of markers that are very tightly linked to a disease gene, an association of an allele (or group of linked alleles) with the disease gene is expected if the disease mutation occurred in the recent past, so that sufficient time has not elapsed for equilibrium to be achieved through recombination events in the specific chromosomal region. When referring to allelic patterns that are comprised of more than one allele, a first allelic pattern is in linkage disequilibrium with a second allelic pattern if all the alleles that comprise the first allelic pattern are in linkage disequilibrium with at least one of the alleles of the second allelic pattern. An example of linkage disequilibrium is that which occurs between the alleles at the IL-1RN (+2018) and IL-1RN (VNTR) polymorphic sites. The two alleles at IL-1RN (+2018) are 100% in linkage disequilIbrium with the two most frequent alleles of IL-1RN (VNTR), which are allele 1 and allele 2.

The term “marker” refers to a sequence in the genome that is known to vary among individuals. For example, the IL-1RN gene has a marker that consists of a variable number of tandem repeats (VNTR).

The term “modulation” as used herein refers to both upregulation (i.e., activation or stimulation (e.g., by agonizing or potentiating)) and downregulation (i.e. inhibition or suppression (e.g., by antagonizing, decreasing or inhibiting)).

The term “mutated gene” refers to an allelic form of a gene, which is capable of altering the phenotype of a subject having the mutated gene relative to a subject which does not have the mutated gene. If a subject must be homozygous for this mutation to have an altered phenotype, the mutation is said to be recessive. If one copy of the mutated gene is sufficient to alter the genotype of the subject, the mutation is said to be dominant. If a subject has one copy of the mutated gene and has a phenotype that is intermediate between that of a homozygous and that of a heterozygous subject (for that gene), the mutation is said to be co-dominant.

The “non-human animals” of the invention include mammalians such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant gene is expressed in some but not all cells of the animal. The term “tissue-specific chimeric animal” indicates that one of the recombinant IL-1L1 genes is present and/or expressed or disrupted in some tissues but not others.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs and as applicable to the embodiment being described, single (sense or antisense) and double-stranded polynucleotides.

The term “nucleotide sequence complementary to the nucleotide sequence set forth in SEQ ID No. x” refers to the nucleotide sequence of the complementary strand of a nucleic acid strand having SEQ ID No. x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID No. x refers to the complementary strand of the strand having SEQ ID No. x or to any nucleic acid having the nucleotide sequence of the complementary strand of SEQ ID No. x. When referring to a single stranded nucleic acid having the nucleotide sequence SEQ ID No. x, the complement of this nucleic acid is a nucleic acid having a nucleotide sequence which is complementary to that of SEQ ID No. x. The nucleotide sequences and complementary sequences thereof are always given in the 5′ to 3′ direction.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Databases with individual sequences are described in Methods in Enzymology, ed. Doolittle, supra. Databases include Genbank, EMBL, and DNA Database of Japan (DDBJ).

Preferred nucleic acids have a sequence at least 70%, and more preferably 80% identical and more preferably 90% and even more preferably at least 95% identical to an nucleic acid sequence of a sequence shown in one of SEQ ID Nos: 1-850. Nucleic acids at least 90%, more preferably 95%, and most preferably at least about 98-99% identical with a nucleic sequence represented in one of SEQ ID Nos: 1-4 are of course also within the scope of the invention. In preferred embodiments, the nucleic acid is mammalian. In comparing a new nucleic acid with known sequences, several alignment tools are available. Examples include PileUp, which creates a multiple sequence alignment, and is described in Feng et al., J. Mol. Evol. (1987) 25:351-360. Another method, GAP, uses the alignment method of Needleman et al., J. Mol. Biol. (1970) 48:443-453. GAP is best suited for global alignment of sequences. A third method, BestFit, functions by inserting gaps to maximize the number of matches using the local homology algorithm of Smith and Waterman, Adv. Appl. Math. (1981) 2:482-489.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long.

A “polymorphic gene” refers to a gene having at least one polymorphic region.

As used herein, the term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

The term “propensity to disease,” also “predisposition” or “susceptibility” to disease or any similar phrase, means that certain IL-1 locus polymorphic alleles are hereby discovered to be associated with or predictive of a particular disease. The alleles are thus over-represented in frequency in individuals with disease as compared to healthy individuals. Thus, these alleles can be used to predict disease even in pre-symptomatic or pre-diseased individuals.

The terms “protein”, “polypeptide” and “peptide” are used interchangeably herein when referring to a gene product.

The term “recombinant protein” refers to a polypeptide of the present invention which is produced by recombinant DNA techniques, wherein generally, DNA encoding an IL-1L1 polypeptide is inserted into a suitable expression vector which is in turn used to transform a host cell to produce the heterologous protein. Moreover, the phrase “derived from”, with respect to a recombinant IL-1L1 gene, is meant to include within the meaning of “recombinant protein” those proteins having an amino acid sequence of a native IL-1L1polypeptide, or an amino acid sequence similar thereto which is generated by mutations including substitutions and deletions (including truncation) of a naturally occurring form of the polypeptide.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention to identify compounds that modulate an IL-1L1 bioactivity.

As used herein, the term “specifically hybridizes” or “specifically detects” refers to the ability of a nucleic acid molecule of the invention to hybridize to at least approximately 6, 12, 20, 30, 50, 100, 150, 200, 300, 350, 400 or 425 consecutive nucleotides of a vertebrate, preferably an IL-1L1 gene.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of one of the IL-1L1genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring forms of IL-1L1 polypeptide.

As used herein, the term “transfection” means the introduction of a nucleic acid, e.g., via an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of an IL-1L1polypeptide or, in the case of anti-sense expression from the transferred gene, the expression of a naturally-occurring form of the IL-1L1 polypeptide is disrupted.

As used herein, the term “transgene” means a nucleic acid sequence (encoding, e.g., one of the IL-1L1polypeptides, or an antisense transcript thereto) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the IL-1L1polypeptides, e.g. either agonistic or antagonistic forms. However, transgenic animals in which the recombinant IL-1L1gene is silent are also contemplated, as for example, the FLP or CRE recombinase dependent constructs described below. Moreover, “transgenic animal” also includes those recombinant animals in which gene disruption of one or more IL-1L1 genes is caused by human intervention, including both recombination and antisense techniques.

The term “treating” as used herein is intended to encompass curing as well as ameliorating at least one symptom of the condition or disease.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

4.3. Nucleic Acids of the Present Invention

The invention provides IL-1L1nucleic acids, homologs thereof, and portions thereof. Preferred nucleic acids have a sequence at least about 60%, 61%, 62%, 63%, 64%, 65%, 66%, 67%, 68%, 69%, 70%, 71%, 72%, 73%, 74%, 75%, 76%, 77%, 78%, 79%, 80%, and more preferably 85% homologous and more preferably 90% and more preferbly 95% and even more preferably at least 99% homologous with a nucleotide sequence of an IL-1L1 gene, e.g., such as a sequence shown in one of SEQ ID Nos: 1, 2, 3, or 4 or complement thereof of the IL-1L1 nucleic acids having ATCC Designation No. XXXXXX or No. XXXXXX. Nucleic acids at least 90%, more preferably 95%, and most preferably at least about 98-99% identical with a nucleic sequence represented in one of SEQ ID Nos. 1, 2, 3, or 4 or complement thereof are of course also within the scope of the invention. In preferred embodiments, the nucleic acid is mammalian and in particularly preferred embodiments, includes all or a portion of the nucleotide sequence corresponding to the coding region such as the nucleic acid set forth in SEQ ID No. 10 or 11 which correspond to the human and murine IL-1L1 ORF sequences contained within the IL-1L1cDNA sequences of SEQ ID Nos. 1 or 4 respectively.

The invention further provides an evolutionarily conserved nucleic acid sequence found in the 3′ UTR (untranslated region) of the IL-1L1 transcript encoded by both human and mouse homologs of the IL-1L1gene. The 41 nucleotide consensus sequence corresponding to this conserved region is 5′-ACAATNAAAANCCCNGATNCTGGTCTCTANTCNCATNAAAA-3′ (SEQ ID No. 12), which is found beginning at nucleotide 1137 of SEQ ID No. 1 (hIL-1L1) and beginning at nucleotide 1146 of SEQ ID No. 4 (mIL-1L1). Typically 5′ and 3′ UTR sequences are not well conserved through evolution because they do not encode functional polypeptide gene product which is subject to selective pressures at the organismal level. However certain such non-coding conserved sequences do occur which play an important role in for example, MRNA processing (e.g. splicing, termination or polyadenylation), mRNA stability or polypeptide translation initiation. Alternatively, such conserved sequences may reflect binding sites for trancriptional regulatory proteins, such as transcriptional activator and repressors, which bind to exonic sequences and affect upstream transcriptional initiation sites. This regulatory sequence can be inserted into heterologous expression systems to affect the level of expression or regulation of, for example, recombinant gene expression. Alternatively, this newly identified sequence may be removed from recombinant forms of the IL-1L1 gene so as to alter the level and/or pattern of IL-1L1 gene expression.

The invention also pertains to isolated nucleic acids comprising a nucleotide sequence encoding IL-1L1polypeptides, variants and/or equivalents of such nucleic acids. The term equivalent is understood to include nucleotide sequences encoding functionally equivalent IL-1L1polypeptides or functionally equivalent peptides having an activity of an IL-1L1protein such as described herein. Equivalent nucleotide sequences will include sequences that differ by one or more nucleotide substitution, addition or deletion, such as allelic variants; and will, therefore, include sequences that differ from the nucleotide sequence of the IL-1L1 gene shown in SEQ ID Nos. 1, 2, 3, or 4 due to the degeneracy of the genetic code.

Preferred nucleic acids are vertebrate IL-1L1 nucleic acids. Particularly preferred vertebrate IL-1L1nucleic acids are mammalian. Regardless of species, particularly preferred IL-1L1 nucleic acids encode polypeptides that are at least 60%, 65%, 70%, 72%, 74%, 76%, 78%, 80%, 90%, or 95% similar or identical to an amino acid sequence of a vertebrate IL-1L1 protein. In one embodiment, the nucleic acid is a cDNA encoding a polypeptide having at least one bio-activity of the subject IL-1L1 polypeptide. Preferably, the nucleic acid includes all or a portion of the nucleotide sequence corresponding to the nucleic acid of SEQ ID Nos. 1, 2, 3 or 4.

Still other preferred nucleic acids of the present invention encode an IL-1L1 polypeptide which is comprised of at least 2, 5, 10, 25, 50, 100, 150 or 200 amino acid residues. For example, such nucleic acids can comprise about 50, 60, 70, 80, 90, or 100 base pairs. Also within the scope of the invention are nucleic acid molecules for use as probes/primer or antisense molecules (i.e. noncoding nucleic acid molecules), which can comprise at least about 6, 12, 20, 30, 50, 60, 70, 80, 90 or 100 base pairs in length.

Another aspect of the invention provides a nucleic acid which hybridizes under stringent conditions to a nucleic acid represented by SEQ ID Nos. 1, 2, 3, or 4 or complement thereof or the nucleic acids having ATCC Designation No. XXXXXX or No. XXXXXX. Appropriate stringency conditions which promote DNA hybridization, for example, 6.0×sodium chloride/sodium citrate (SSC) at about 45 [symbol] C., followed by a wash of 2.0×SSC at 50 [symbol] C., are known to those skilled in the art or can be found in Current Protocols in Molecular Biology, John Wiley & Sons, N.Y. (1989), 6.3.1-6.3.6 or in Molecular Cloning: A Laboratory Manual, Cold Spring Harbor Press (1989). For example, the salt concentration in the wash step can be selected from a low stringency of about 2.0×SSC at 50 [symbol] C. to a high stringency of about 0.2×SSC at 50 [symbol] C. In addition, the temperature in the wash step can be increased from low stringency conditions at room temperature, about 22 [symbol] C., to high stringency conditions at about 65 [symbol] C. Both temperature and salt may be varied, or temperature and salt concentration may be held constant while the other variable is changed. In a preferred embodiment, an IL-1L1nucleic acid of the present invention will bind to one of SEQ ID Nos. 1, 2, 3, or 4 or complement thereof under moderately stringent conditions, for example at about 2.0×SSC and about 40o C. In a particularly preferred embodiment, an IL-1L1nucleic acid of the present invention will bind to one of SEQ ID Nos. 1, 2, 3, or 4 or complement thereof under high stringency conditions. In another particularly preferred embodiment, an IL-1L1 nucleic acid sequence of the present invention will bind to one of SEQ ID Nos. 10 or 11, which correspond to IL-1L1 ORF nucleic acid sequences, under high stringency conditions.

Nucleic acids having a sequence that differs from the nucleotide sequences shown in one of SEQ ID Nos. 1 or 4, or 10 or 11 or complement thereof due to degeneracy in the genetic code are also within the scope of the invention. Such nucleic acids encode functionally equivalent peptides (i.e., peptides having a biological activity of an IL-1L1 polypeptide) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of an IL-1L1 polypeptide. However, it is expected that DNA sequence polymorphisms that do lead to changes in the amino acid sequences of the subject IL-1L1polypeptides will exist among mammals. One skilled in the art will appreciate that these variations in one or more nucleotides (e.g., up to about 3-5% of the nucleotides) of the nucleic acids encoding polypeptides having an activity of an IL-1L1polypeptide may exist among individuals of a given species due to natural allelic variation.

4.3.1 Probes and Primers

The nucleotide sequences determined from the cloning of IL-1L1genes from mammalian organisms will further allow for the generation of probes and primers designed for use in identifying and/or cloning IL-1L1 homologs in other cell types, e.g., from other tissues, as well as IL-1L1homologs from other mammalian organisms. For instance, the present invention also provides a probe/primer comprising a substantially purified oligonucleotide, which oligonucleotide comprises a region of nucleotide sequence that hybridizes under stringent conditions to at least approximately 12, preferably 25, more preferably 40, 50 or 75 consecutive nucleotides of sense or anti-sense sequence selected from the group consisting of SEQ ID Nos. 1, 2, 3, 4, 10 or 11 or naturally occurring mutants thereof. For instance, primers based on the nucleic acid represented in SEQ ID Nos. 10 or 11 can be used in PCR reactions to clone IL-1L1 polypeptide encoding genes.

In preferred embodiments, the IL-1L1primers are designed so as to optimize specificity and avoid secondary structures which affect the efficiency of priming. Optimized PCR primers of the present invention are designed so that “upstream” and “downstream” primers have approximately equal melting temperatures such as can be estimated using the formulae: Tₘ=81.5 [symbol] C−16.6(log₁₀[Na⁺])+0.41(% G+C)−0.63 (% formamide)−(600/length); or Tₘ( [symbol] C)=2(A/T)+4(G/C). Optimized IL-1L1 primers may also be designed by using various programs, such as “Primer3” provided by the Whitehead Institute for Biomedical Research at http://www-genome.wi.mit.edu/cgi-bin/primer/primer3.cgi.

In preferred embodiments, the IL-1L1probes and primers can be used to detect human IL-1 locus polymorphisms which occur within and surrounding the IL-1L1gene sequence. Genetic variations within the IL-1 locus may be associated with the likelihood of the development of a number of human diseases and conditions, such as inflammatory and autoimmune diseases in which IL-1 encoded polypeptides play an important etiological role. Accordingly the invention provides probes and primers for IL-1 locus polymorphisms, including polymorphisms associated with the human and mouse IL-1L1 gene. PCR primers of the invention include those which flank an IL-1L1 human polymorphism and allow amplification and analysis of this region of the genome. Analysis of polymorphic allele identity may be conducted, for example, by direct sequencing or by the use of allele-specific capture probes or by the use of molecular beacon probes. Alternatively, the polymorphic allele may allow for direct detection by the creation or elimination of a restriction endonuclease recognition site(s) within the PCR product or after an appropriate sequence modification is designed into at least one of the primers such that the altered sequence of the primer, when incorporated into the PCR product resulting from amplification of a specific IL-1 polymorphic allele, creates a unique restriction site in combination with at least one allele but not with at least one other allele of that polymorphism. IL-1 polymorphisms corresponding to variable number of tandem repeat (VNTR) polymorphisms may be detected by the electrophoretic mobility and hence size of a PCR product obtained using primers which flank the VNTR. Still other IL-1 polymorphisms corresponding to restriction fragment length polymorphisms (RFLPs) may be detected directly by the mobility of bands on a Southern blot using appropriate IL-1 locus probes and genomic DNA or cDNA obtained from an appropriate sample organism such as a human or a non-human animal.

Likewise, probes based on the subject IL-1L1 sequences can be used to detect transcripts or genomic sequences encoding the same or homologous proteins, for use, e.g, in prognostic or diagnostic assays (further described below). The invention provides probes which are common to alternatively spliced variants of the IL-1L1 transcript, such as those corresponding to at least 12 consecutive nucleotides complementary to a sequence found in any of SEQ ID Nos. 1, 4, 10 or 11. In addition, the invention provides probes which hybridize specifically to alternatively spliced forms of the IL-1L1transcript. For example, probes comprising at least 12 consecutive nucleotides complementary to a sequence found in any of SEQ ID Nos. 2 or 3 can be used to detect alternatively spliced forms of the human IL-1L1 gene transcript corresponding to a form of the transcript having alternative 5′ end #1 or alternative end #2 respectively (FIG. 1). In preferred embodiments, the probe further comprises a label group attached thereto and able to be detected, e.g., the label group is selected from amongst radioisotopes, fluorescent compounds, enzymes, and enzyme co-factors.

Probes and primers can be prepared and modified, e.g., as previously described herein for other types of nucleic acids.

4.3.2 Antisense, Ribozyme and Triplex Techniques

Another aspect of the invention relates to the use of the isolated nucleic acid in “antisense” therapy. As used herein, “antisense” therapy refers to administration or in situ generation of oligonucleotide molecules or their derivatives which specifically hybridize (e.g., bind) under cellular conditions, with the cellular mRNA and/or genomic DNA encoding one or more of the subject IL-1L1proteins so as to inhibit expression of that protein, e.g., by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes an IL-1L1 protein. Alternatively, the antisense construct is an oligonucleotide probe which is generated ex vivo and which, when introduced into the cell causes inhibition of expression by hybridizing with the mRNA and/or genomic sequences of an IL-1L1gene. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668. With respect to antisense DNA, oligodeoxyribonucleotides derived from the translation initiation site, e.g., between the −10 and +10 regions of the IL-1L1 nucleotide sequence of interest, are preferred.

Antisense approaches involve the design of oligonucleotides (either DNA or RNA) that are complementary to IL-1L1 mRNA. The antisense oligonucleotides will bind to the IL-1L1 mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of an IL-1L1gene could be used in an antisense approach to inhibit translation of endogenous IL-1L1 mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the invention. Whether designed to hybridize to the 5′, 3′ or coding region of IL-1L1mRNA, antisense nucleic acids should be at least six nucleotides in length, and are preferably less than about 100 and more preferably less than about 50, 25, 17 or 10 nucleotides in length.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their ability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a fonnacetal or analog thereof.

In yet a further embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate olgonucleotides can be prepared by use of controlled pore glass polymer supports (Sarin et al., 1988, Proc. Natl. Acad. Sci. U.S.A. 85:7448-7451), etc.

While antisense nucleotides complementary to the IL-1L1coding region sequence can be used, those complementary to the transcribed untranslated region and to the region comprising the initiating methionine are most preferred.

The antisense molecules can be delivered to cells which express IL-1L1 in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous IL-1L1transcripts and thereby prevent translation of the IL-1L1mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive and can include but not be limited to: the SV40 early promoter region (Bemoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct which can be introduced directly into the tissue site. Alternatively, viral vectors can be used which selectively infect the desired tissue, in which case administration may be accomplished by another route (e.g., systematically).

Ribozyme molecules designed to catalytically cleave IL-1L1mRNA transcripts can also be used to prevent translation of IL-1L1 mRNA and expression of IL-1L1 (See, e.g., PCT International Publication WO90/11364, published Oct. 4, 1990; Sarver et al., 1990, Science 247:1222-1225 and U.S. Pat. No. 5,093,246). While ribozymes that cleave mRNA at site specific recognition sequences can be used to destroy IL-1L1mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. There are a number of potential hammerhead ribozyme cleavage sites within the nucleotide sequence of human IL-1L1cDNA (FIG. 1) and the murine IL-1L1cDNA (FIG. 2). Preferably the ribozyme is engineered so that the cleavage recognition site is located near the 5′ end of the IL-1L1mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in an IL-1L1gene.

As in the antisense approach, the ribozymes can be composed of modified oligonucleotides (e.g., for improved stability, targeting, etc.) and should be delivered to cells which express the IL-1L1gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous IL-1L1 messages and inhibit translation. Because ribozymes unlike antisense molecules, are catalytic, a lower intracellular concentration is required for efficiency.

Endogenous IL-1L1gene expression can also be reduced by inactivating or “knocking out” the IL-1L1gene or its promoter using targeted homologous recombination. (E.g., see Smithies et al., 1985, Nature 317:230-234; Thomas & Capecchi, 1987, Cell 51:503-512; Thompson et al., 1989 Cell 5:313-321; each of which is incorporated by reference herein in its entirety). For example, a mutant, non-functional IL-1L1 (or a completely unrelated DNA sequence) flanked by DNA homologous to the endogenous IL-1L1 gene (either the coding regions or regulatory regions of the IL-1L1 gene) can be used, with or without a selectable marker and/or a negative selectable marker, to transfect cells that express IL-1L1 in vivo. Insertion of the DNA construct, via targeted homologous recombination, results in inactivation of the IL-1L1gene. Such approaches are particularly suited in the agricultural field where modifications to ES (embryonic stem) cells can be used to generate animal offspring with an inactive IL-1L1 (e.g., see Thomas & Capecchi 1987 and Thompson 1989, supra). However this approach can be adapted for use in humans provided the recombinant DNA constructs are directly administered or targeted to the required site in vivo using appropriate viral vectors.

Alternatively, endogenous IL-1L1gene expression can be reduced by targeting deoxyribonucleotide sequences complementary to the regulatory region of the IL-1L1 gene (i.e., the IL-1L1 promoter and/or enhancers) to form triple helical structures that prevent transcription of the IL-1L1 gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

4.3.3. Vectors Encoding IL-1L1 Proteins and IL-1L1 Expressing Cells

The invention further provides plasmids and vectors encoding an IL-1L1 protein, which can be used to express an IL-1L1 protein in a host cell. The host cell may be any prokaryotic or eukaryotic cell. Thus, a nucleotide sequence derived from the cloning of mammalian IL-1L1 proteins, encoding all or a selected portion of the full-length protein, can be used to produce a recombinant form of an IL-1L1polypeptide via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial) cells, are standard procedures well known in the art.

Vectors that allow expression of a nucleic acid in a cell are referred to as expression vectors. Typically, expression vectors used for expressing an IL-1L1protein contain a nucleic acid encoding an IL-1L1polypeptide, operably linked to at least one transcriptional regulatory sequence. Regulatory sequences are art-recognized and are selected to direct expression of the subject IL-1L1 proteins. Transcriptional regulatory sequences are described in Goeddel; Gene Expression Technology: Methods in Enzymology 185, Academic Press, San Diego, Calif. (1990). In one embodiment, the expression vector includes a recombinant gene encoding a peptide having an agonistic activity of a subject IL-1L1 polypeptide, or alternatively, encoding a peptide which is an antagonistic form of an IL-1L1 protein.

Suitable vectors for the expression of an IL-1L1polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used. In an illustrative embodiment, an IL-1L1polypeptide is produced recombinantly utilizing an expression vector generated by sub-cloning the coding sequence of one of the IL-1L1genes represented in SEQ ID Nos. 1 or 4.

The preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2^{nd} Ed., ed. by Sambrook, Fritsch and Maniatis (Cold Spring Harbor Laboratory Press: 1989) Chapters 16 and 17.

In some instances, it may be desirable to express the recombinant IL-1L1 polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III)

When it is desirable to express only a portion of an IL-1L1protein, such as a form lacking a portion of the N-terminus, i.e. a truncation mutant which lacks the signal peptide, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al. (1987) J. Bacteriol. 169:751-757) and Salmonella typhimurium and its in vitro activity has been demonstrated on recombinant proteins (Miller et al. (1987) PNAS 84:2718-1722). Therefore, removal of an N-terminal methionine, if desired, can be achieved either in vivo by expressing IL-1L1 derived polypeptides in a host which produces MAP (e.g., E. coli or CM89 or S. cerevisiae), or in vitro by use of purified MAP (e.g., procedure of Miller et al., supra).

Moreover, the gene constructs of the present invention can also be used as part of a gene therapy protocol to deliver nucleic acids encoding either an agonistic or antagonistic form of one of the subject IL-1L1 proteins. Thus, another aspect of the invention features expression vectors for in vivo or in vitro transfection and expression of an IL-1L1 polypeptide in particular cell types so as to reconstitute the function of, or alternatively, abrogate the function of IL-1L1 in a tissue. This could be desirable, for example, when the naturally-occurring form of the protein is misexpressed or the natural protein is mutated and less active.

In addition to viral transfer methods, non-viral methods can also be employed to cause expression of a subject IL-1L1 polypeptide in the tissue of an animal. Most nonviral methods of gene transfer rely on normal mechanisms used by mammalian cells for the uptake and intracellular transport of macromolecules. In preferred embodiments, non-viral targeting means of the present invention rely on endocytic pathways for the uptake of the subject IL-1L1 polypeptide gene by the targeted cell. Exemplary targeting means of this type include liposomal derived systems, poly-lysine conjugates, and artificial viral envelopes.

In other embodiments transgenic animals, described in more detail below could be used to produce recombinant proteins.

4.4. Polypeptides of the Present Invention

The present invention makes available isolated IL-1L1 polypeptides which are isolated from, or otherwise substantially free of other cellular proteins. The term “substantially free of other cellular proteins” (also referred to herein as “contaminating proteins”) or “substantially pure or purified preparations” are defined as encompassing preparations of IL-1L1polypeptides having less than about 20% (by dry weight) contaminating protein, and preferably having less than about 5% contaminating protein. Functional forms of the subject polypeptides can be prepared, for the first time, as purified preparations by using a cloned gene as described herein.

Preferred IL-1L1proteins of the invention have an amino acid sequence which is at least about 60%, 65%, 66%, 67%, 68%, 69%, 70%, 71%, 72%, 73%, 74%, 75%, 76%, 77%, 78%, 79%, 80%, 85%, 90%, or 95% identical or homologous to an amino acid sequence of SEQ ID No. 5 or 6. Even more preferred IL-1L1proteins comprise an amino acid sequence of at least 10, 20, 30, or 50 residues which is at least about 70, 80, 90, 95, 97, 98, or 99% homologous or identical to an amino acid sequence of SEQ ID Nos. 5 or 6. Such proteins can be recombinant proteins, and can be, e.g., produced in vitro from nucleic acids comprising a nucleotide sequence set forth in SEQ ID Nos. 1, 4, 10 or 11, or homologs thereof. For example, recombinant polypeptides preferred by the present invention can be encoded by a nucleic acid, which is at least 85% homologous and more preferably 90% homologous and most preferably 95% homologous with a nucleotide sequence set forth in SEQ ID Nos. 1, 4, 10 or 11. Polypeptides which are encoded by a nucleic acid that is at least about 98-99% homologous with the sequence of SEQ ID Nos. 1, 4, 10 or 11 are also within the scope of the invention.

In a preferred embodiment, an IL-1L1protein of the present invention is a mammalian IL-1L1 protein. In a particularly preferred embodiment an IL-1L1 protein is set forth as SEQ ID No. 5 or SEQ ID No. 6. In particularly preferred embodiments, an IL-1L1 protein has an IL-1L1 bioactivity. It will be understood that certain post-translational modifications, e.g., phosphorylation and the like, can increase the apparent molecular weight of the IL-1L1 protein relative to the unmodified polypeptide chain.

The invention also features protein isoforms encoded by splice variants of the present invention. Such isoforms may have biological activities identical to or different from those possessed by the IL-1L1 proteins specified by SEQ ID Nos. 5 or 6. Such isoforms may arise, for example, by alternative splicing of one or more IL-1L1gene transcripts.

IL-1L1polypeptides preferably are capable of functioning as either an agonist or antagonist of at least one biological activity of a wild-type (“authentic”) IL-1L1 protein of the appended sequence listing. The term “evolutionarily related to”, with respect to amino acid sequences of IL-1L1 proteins, refers to both polypeptides having amino acid sequences which have arisen naturally, and also to mutational variants of human IL-1L1polypeptides which are derived, for example, by combinatorial mutagenesis.

Full length proteins or fragments corresponding to one or more particular motifs and/or domains or to arbitrary sizes, for example, at least 5, 10, 20, 25, 50, 75 and 100, amino acids in length are within the scope of the present invention.

For example, isolated IL-1L1 polypeptides can be encoded by all or a portion of a nucleic acid sequence shown in any of SEQ ID Nos. 1, 4, 10 or 11. Isolated peptidyl portions of IL-1L1 proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, an IL-1L1 polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (e.g., “authentic”) IL-1L1protein.

An IL-1L1polypeptide can be a membrane bound form or a soluble form. A preferred soluble IL-1L1 polypeptide is a polypeptide which does not contain a hydrophobic signal sequence domain. Such proteins can be created by genetic engineering by methods known in the art. The solubility of a recombinant polypeptide may be increased by deletion of hydrophobic domains, such as predicted transmembrane domains, of the wild type protein. For example, the SMART (Simple Modular Architecture Research Tool, v3.0; Schultz et al. (1998) Proc. Natl. Acad. Sci. USA 95: 5857-5864) predicts that the huIL-1L1 polypeptide sequence extending from amino acid residue 53 to amino acid residue 73 encodes a transmembrane segment having the sequence LSPVILGVQGGSQCLSCGVG. Thus, soluble IL-1L1proteins can comprise an amino acid sequence of about amino acid 74 to amino acid 155 of SEQ ID No. 5 or 6. A second IL-1L1 transmembrane segment is predicted for the sequence GLTSSFESAAYPGWFLCTVP extending from amino acid residue 105 to amino acid residue 125. Accordingly, certain soluble forms of recombinant IL-1L1include those carrying deletions of this domain such as IL-1L1polypeptides comprising amino acids 1 to 105 of SEQ ID No. 5 or 6. Alternatively, both predicted transmembrane regions may be deleted and/or sequences just distal to these domains recombined into the recombinant form. For example, soluble recombinant IL-1L1polypeptides comprising residues 1 to 52, 74 to 104 and 126 to 155 of SEQ ID Nos. 5 or 6 are contemplated. A leaderless recombinant interleukin-1 (IL-1) receptor antagonist missing its native signal sequence accumulated in an osmotically sensitive cellular compartment when expressed recombinantly in E. coli (Thostenson et al. (1997) J Bacteriol 179: 5333-9). Accordingly, analogous recombinant forms of the IL-1L1polypeptide, optimized for the recovery of soluble native polypeptide, are within the scope of the instant invention.

In general, polypeptides referred to herein as having an activity (e.g., are “bioactive”) of an IL-1L1 protein are defined as polypeptides which include an amino acid sequence encoded by all or a portion of the nucleic acid sequences shown in one of SEQ ID No. 1, 4, 10 or 11 and which mimic or antagonize all or a portion of the biological/biochemical activities of a naturally occurring IL-1L1protein. Examples of such biological activity include a region of conserved structure referred to as the IL1 domain (Dinarello (1997) Cytokine Growth Factor Rev. 8: 253-65; Patarca et al. (1997) Crit Rev Oncol 8: 142-88; Kurzrock et al. (1995) Cytokines Mol Ther 1: 177-84) which extends from amino acid residue 4 to amino acid residue 152 of SEQ ID No. 5 or 6. This domain comprises regions of particular homology to the IL-1 agonist IL-1b including the IL-1L1 agonist consensus sequences: LKXLXL extending from residue 16 to residue 21 of SEQ ID Nos. 5 and 6; FESA extending from residue 111 to 114 of SEQ ID Nos. 5 and 6; and and ITDFXXQ extending from residue 146 to residue 152 of SEQ ID Nos. 5 and 6. IL-1L1 polypeptides of the instant invention further comprise regions of conserved sequence found within the IL-1 antagonist IL-1ra polypeptide. For example, conserved IL-1 antagonist polypeptides include the IL-1L1/IL-1ra consensus sequences: YLXNNQLXAGXL extending from residue 20 to residue 31 of SEQ ID No. 5 or 6; LEXVNIXXL extending from residue 80 to residue 88 of SEQ ID No. 5 or 6; and TXSFESAAXPGWFLCTXXEADQPV extending from residue 108 to residue 131 of SEQ ID No. 5 or 6. Structural comparison of IL-1L1, agonist IL-1b, and antagonist IL-1ra polypeptides further reveal the presence of an extensive amino-terminal acid domain extending from residue 1 to residue 140 of the human interleukin-1 beta precursor (GenBank Accession No. P01584) which is absent from the IL-1ra polypeptide as well as the IL-1L1 polypeptide of SEQ ID No. 5 or 6. The absence of this IL-1 agonist domain is therefore associated with IL-1 antagonist polypeptides. Recombinant IL-1L1polypeptides of the present invention include those comprising the sequences 1 to 140 of the human interleukin-1 beta precursor (GenBank Accession No. P01584) fused to an IL-1L1 polypeptide sequence comprising one or more segments of the IL-1L1polypeptides of SEQ ID No. 5 or 6.

Still other structural features of the IL-1L1polypeptides include glycosylation consensus sequences such as the myristylation sequences: GGLHAG extending from residue 29 to residue 34 of SEQ ID No. 5; GLHAGK extending from residue 30 to residue 35 of SEQ ID No. 5; GVQGGS extending from residue 60 to residue 65 of SEQ ID No. 5; GGSQCL extending from residue 63 to residue 68 of SEQ ID No. 5; GQEPTL extending from residue 73 to residue 78 of SEQ ID No. 5; GAKESK extending from residue 91 to residue 96 of SEQ ID No. 5; and GLTSSF extending from residue 106 to residue 111 of SEQ ID No. 5. In addition, the IL-1L1polypeptides of the present invention include two casein kinase 2 phosphorylation sites: SSFE extending from residue 109 to residue 112 of SEQ ID No. 5; and TVPE extending from residue 123 to residue 126 of SEQ ID No. 5.

Other biological activities of the subject IL-1L1 proteins will be reasonably apparent to those skilled in the art. According to the present invention, a polypeptide has biological activity if it is a specific agonist or antagonist of a naturally-occurring form of an IL-1L1 protein.

Assays for determining whether a compound, e.g, a protein, such as an IL-1L1 protein or variant thereof, has one or more of the above biological activities include those assays, well known in the art, which are used for assessing IL-1 agonist and IL-1 antagonist activities. For example, the ability of recombinant IL-1L1polypeptide to activate the expression of interleukin-6 gene expression in human skin fibroblasts is indicative of an IL-1b-like IL-1 agonist activity. In contrast, the ability of recombinant IL-1L1polypeptides to interfere with IL-1a or IL-1b induced activation of interleukin-6 gene expression is indicative of IL-1ra-like IL-1 antagonist activity. Optimally, such assays may be conducted in primary human umbilical vein endothelial cells as the IL-1L1 gene is known to be highly expressed in human placenta and, accordingly, IL-1L1-specific receptors are likely present in such tissues.

Other preferred proteins of the invention are those encoded by the nucleic acids set forth in the section pertaining to nucleic acids of the invention. In particular, the invention provides fusion proteins, e.g., IL-1L1-immunoglobulin fusion proteins. Such fusion proteins can provide, e.g., enhanced stability and solubility of IL-1L1proteins and may thus be useful in therapy. Fusion proteins can also be used to produce an immunogenic fragment of an IL-1L1 protein. For example, the VP6 capsid protein of rotavirus can be used as an immunologic carrier protein for portions of the IL-1L1polypeptide, either in the monomeric form or in the form of a viral particle. The nucleic acid sequences corresponding to the portion of a subject IL-1L1protein to which antibodies are to be raised can be incorporated into a fusion gene construct which includes coding sequences for a late vaccinia virus structural protein to produce a set of recombinant viruses expressing fusion proteins comprising IL-1L1epitopes as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of an IL-1L1protein and the poliovirus capsid protein can be created to enhance immunogenicity of the set of polypeptide antigens (see, for example, EP Publication No: 0259149; and Evans et al. (1989) Nature 339:385; Huang et al. (1988) J. Virol. 62:3855; and Schlienger et al. (1992) J. Virol. 66:2).

The Multiple antigen peptide system for peptide-based immunization can also be utilized to generate an immunogen, wherein a desired portion of an IL-1L1 polypeptide is obtained directly from organo-chemical synthesis of the peptide onto an oligomeric branching lysine core (see, for example, Posnett et al. (1988) JBC 263:1719 and Nardelli et al. (1992) J. Immunol. 148:914). Antigenic determinants of IL-1L1proteins can also be expressed and presented by bacterial cells.

In addition to utilizing fusion proteins to enhance immunogenicity, it is widely appreciated that fusion proteins can also facilitate the expression of proteins, and accordingly, can be used in the expression of the IL-1L1polypeptides of the present invention. For example, IL-1L1polypeptides can be generated as glutathione-S-transferase (GST-fusion) proteins. Such GST-fusion proteins can enable easy purification of the IL-1L1polypeptide, as for example by the use of glutathione-derivatized matrices (see, for example, Current Protocols in Molecular Biology, eds. Ausubel et al. (N.Y.: John Wiley & Sons, 1991)). Additionally, fusion of IL-1L1polypeptides to small epitope tags, such as the FLAG or hemagluttinin tag sequences, can be used to simplify immunological purification of the resulting recombinant polypeptide or to facilitate immunological detection in a cell or tissue sample. Fusion to the green fluorescent protein, and recombinant versions thereof which are known in the art and available commercially, may further be used to localize IL-1L1 polypeptides within living cells and tissue.

The present invention further pertains to methods of producing the subject IL-1L1 polypeptides. For example, a host cell transfected with a nucleic acid vector directing expression of a nucleotide sequence encoding the subject polypeptides can be cultured under appropriate conditions to allow expression of the peptide to occur. Suitable media for cell culture are well known in the art. The recombinant IL-1L1polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant IL-1L1 polypeptide is a fusion protein containing a domain which facilitates its purification, such as GST fusion protein.

Moreover, it will be generally appreciated that, under certain circumstances, it may be advantageous to provide homologs of one of the subject IL-1L1polypeptides which function in a limited capacity as one of either an IL-1L1 agonist (mimetic) or an IL-1L1 antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of IL-1L1 proteins.

Homologs of each of the subject IL-1L1 proteins can be generated by mutagenesis, such as by discrete point mutation(s), or by truncation. For instance, mutation can give rise to homologs which retain substantially the same, or merely a subset, of the biological activity of the IL-1L1 polypeptide from which it was derived. Alternatively, antagonistic forms of the protein can be generated which are able to inhibit the function of the naturally occurring form of the protein, such as by competitively binding to an IL-1L1 receptor.

The recombinant IL-1L1polypeptides of the present invention also include homologs of the wildtype IL-1L1proteins, such as versions of those protein which are resistant to proteolytic cleavage, as for example, due to mutations which alter ubiquitination or other enzymatic targeting associated with the protein.

IL-1L1 polypeptides may also be chemically modified to create IL-1L1 derivatives by forming covalent or aggregate conjugates with other chemical moieties, such as glycosyl groups, lipids, phosphate, acetyl groups and the like. Covalent derivatives of IL-1L1 proteins can be prepared by linking the chemical moieties to functional groups on amino acid sidechains of the protein or at the N-terminus or at the C-terminus of the polypeptide.

Modification of the structure of the subject IL-1L1 polypeptides can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally-occurring form of the protein, or to produce specific antagonists thereof, are considered functional equivalents of the IL-1L1polypeptides described in more detail herein. Such modified peptides can be produced, for instance, by amino acid substitution, deletion, or addition. The substitutional variant may be a substituted conserved amino acid or a substituted non-conserved amino acid.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2^{nd} ed., Ed. by L. Stryer, WH Freeman and Co.: 1981). Whether a change in the amino acid sequence of a peptide results in a functional IL-1L1 homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

This invention further contemplates a method for generating sets of combinatorial mutants of the subject IL-1L1proteins as well as truncation mutants, and is especially useful for identifying potential variant sequences (e.g., homologs). The purpose of screening such combinatorial libraries is to generate, for example, novel IL-1L1homologs which can act as either agonists or antagonist, or alternatively, possess novel activities all together. Thus, combinatorially-derived homologs can be generated to have an increased potency relative to a naturally occurring form of the protein.

In one embodiment, the variegated 1IL-1L1ary of IL-1L1variants is generated by combinatorial mutagenesis at the nucleic acid level, and is encoded by a variegated gene 1IL-1L1ary. For instance, a mixture of synthetic oligonucleotides can be enzymatically ligated into gene sequences such that the degenerate set of potential IL-1L1sequences are expressible as individual polypeptides, or alternatively, as a set of larger fusion proteins (e.g., for phage display) containing the set of IL-1L1 sequences therein.

There are many ways by which such libraries of potential IL-1L1homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential IL-1L1sequences. The synthesis of degenerate oligonucleotides is well known in the art (see for example, Narang, S A (1983) Tetrahedron 39:3; Itakura et al. (1981) Recombinant DNA, Proc 3^{rd} Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Likewise, a library of coding sequence fragments can be provided for an IL-1L1 clone in order to generate a variegated population of IL-1L1fragments for screening and subsequent selection of bioactive fragments. A variety of techniques are known in the art for generating such 1, including chemical synthesis. In one embodiment, a library of coding sequence fragments can be generated by (i) treating a double stranded PCR fragment of an IL-1L1 coding sequence with a nuclease under conditions wherein nicking occurs only about once per molecule; (ii) denaturing the double stranded DNA; (iii) renaturing the DNA to form double stranded DNA which can include sense/antisense pairs from different nicked products; (iv) removing single stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of IL-1L1homologs. The most widely used techniques for screening large gene libraries typically comprises cloning the gene library into replicable expression vectors, transforming appropriate cells with the resulting libraries of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate IL-1L1sequences created by combinatorial mutagenesis techniques. Combinatorial mutagenesis has a potential to generate very large libraries of mutant proteins, e.g., in the order of 1026 molecules. Combinatorial libraries of this size may be technically challenging to screen even with high throughput screening assays. To overcome this problem, a new technique has been developed recently, recrusive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (Arkin and Yourvan, 1992, PNAS USA 89:7811-7815; Yourvan et al., 1992, Parallel Problem Solving from Nature, 2., In Maenner and Manderick, eds., Elsevir Publishing Co., Amsterdam, pp. 401-410; Delgrave et al., 1993, Protein Engineering 6(3):327-331).

The invention also provides for reduction of the IL-1L1proteins to generate mimetics, e.g., peptide or non-peptide agents, such as small molecules, which are able to disrupt binding of an IL-1L1 polypeptide of the present invention with a molecule, e.g. target peptide. Thus, such mutagenic techniques as described above are also useful to map the determinants of the IL-1L1 proteins which participate in protein-protein interactions involved in, for example, binding of the subject IL-1L1 polypeptide to a target peptide. To illustrate, the critical residues of a subject IL-1L1 polypeptide which are involved in molecular recognition of its receptor can be determined and used to generate IL-1L1 derived peptidomimetics or small molecules which competitively inhibit binding of the authentic IL-1L1 protein with that moiety. By employing, for example, scanning mutagenesis to map the amino acid residues of the subject IL-1L1 proteins which are involved in binding other proteins, peptidomimetic compounds can be generated which mimic those residues of the IL-1L1 protein which facilitate the interaction. Such mimetics may then be used to interfere with the normal function of an IL-1L1 protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9^{th} American Peptide Symposium) Pierce Chemical Co. Rockland, Ill., 1985), b-turn dipeptide cores (Nagai et al. (1985) Tetrahedron Lett 26:647; and Sato et al. (1986) J Chem Soc Perkin Trans 1:1231), and b-aminoalcohols (Gordon et al. (1985) Biochem Biophys Res Commun126:419; and Dann et al. (1986) Biochem Biophys Res Commun 134:71).

4.5. Anti-IL-1L1 Antibodies and Uses Therefor

Another aspect of the invention pertains to an antibody specifically reactive with a mammalian IL-1L1 protein, e.g., a wild-type or mutated IL-1L1 protein. For example, by using immunogens derived from an IL-1L1 protein, e.g., based on the cDNA sequences, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a mammalian IL-1L1 polypeptide or an antigenic fragment which is capable of eliciting an antibody response, or a fusion protein as described above). Techniques for conferring immunogenicity on a protein or peptide include conjugation to carriers or other techniques well known in the art. An immunogenic portion of an IL-1L1protein can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of an IL-1L1 protein of a mammal, e.g., antigenic determinants of a protein set forth in SEQ ID No. 5 or 6 or closely related homologs (e.g., at least 90% homologous, and more preferably at least 94% homologous).

Following immunization of an animal with an antigenic preparation of an IL-1L1 polypeptide, anti-IL-1L1antisera can be obtained and, if desired, polyclonal anti-IL-1L1 antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique originally developed by Kohler and Milstein ((1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian IL-1L1polypeptide of the present invention and monoclonal antibodies isolated from a culture comprising such hybridoma cells. In one embodiment anti-human IL-1L1antibodies specifically react with the protein encoded by a nucleic acid having SEQ ID No. 1, 4, 10 or 11.

The term antibody as used herein is intended to include fragments thereof which are also specifically reactive with one of the subject mammalian IL-1L1polypeptides. Antibodies can be fragmented using conventional techniques and the fragments screened for utility in the same manner as described above for whole antibodies. For example, F(ab)2 fragments can be generated by treating antibody with pepsin. The resulting F(ab)2 fragment can be treated to reduce disulfide bridges to produce Fab fragments. The antibody of the present invention is further intended to include bispecific, single-chain, and chimeric and humanized molecules having affinity for an IL-1L1protein conferred by at least one CDR region of the antibody. In preferred embodiments, the antibody further comprises a label attached thereto and able to be detected, (e.g., the label can be a radioisotope, fluorescent compound, enzyme or enzyme co-factor).

Anti-IL-1L1 antibodies can be used, e.g., to monitor IL-1L1protein levels in an individual for determining, e.g., whether a subject has a disease or condition associated with an aberrant IL-1L1protein level, or allowing determination of the efficacy of a given treatment regimen for an individual afflicted with such a disorder. The level of IL-1L1 polypeptides may be measured from cells in bodily fluid, such as in blood samples.

Another application of anti-IL-1L1 antibodies of the present invention is in the immunological screening of cDNA libraries constructed in expression vectors such as λgt11, λgt18-23, λZAP, and λORF8. Messenger libraries of this type, having coding sequences inserted in the correct reading frame and orientation, can produce fusion proteins. For instance, λgt11 will produce fusion proteins whose amino termini consist of β-galactosidase amino acid sequences and whose carboxy termini consist of a foreign polypeptide. Antigenic epitopes of an IL-1L1protein, e.g., other orthologs of a particular IL-1L1 protein or other paralogs from the same species, can then be detected with antibodies, as, for example, reacting nitrocellulose filters lifted from infected plates with anti-IL-1L1 antibodies. Positive phage detected by this assay can then be isolated from the infected plate. Thus, the presence of IL-1L1 homologs can be detected and cloned from other animals, as can alternate isoforms (including splice variants) from humans.

4.6. Transgenic Animals

The invention further provides for transgenic animals, which can be used for a variety of purposes, e.g., to identify IL-1L1 therapeutics. Transgenic animals of the invention include non-human animals containing a heterologous IL-1L1 gene or fragment thereof under the control of an IL-1L1promoter or under the control of a heterologous promoter. Accordingly, the transgenic animals of the invention can be animals expressing a transgene encoding a wild-type IL-1L1protein or fragment thereof or variants thereof, including mutants and polymorphic variants thereof. Such animals can be used, e.g., to determine the effect of a difference in amino acid sequence of an IL-1L1 protein from the sequence set forth in SEQ ID Nos. 5 or 6, such as a polymorphic difference. These animals can also be used to determine the effect of expression of an IL-1L1 protein in a specific site or for identifying IL-1L1 therapeutics or confirming their activity in vivo.

In one aspect, the invention provides transgenic non-human organisms and cell lines for use in the in vivo screening and evaluation of drugs or other therapeutic regimens useful in the treatment of inflammatory disorders. In one embodiment, the invention is a transgenic animal with a targeted disruption in an interleukin-1 gene. In particular, the gene is the IL-1L1gene. The animal may be chimeric, heterozygotic or homozygotic for the disrupted gene. Homozygotic knock-out IL-1L1mammals provide a model for studying inflammatory conditions, such as rheumatoid arthritis, inflammatory bowel disorder, Type I diabetes, psoriasis, osteoporosis, nephropathy in diabetes mellitus, alopecia areata, Graves disease, systemic lupus erythematosus, lichen sclerosis, ulcerative colitis, coronary artery disease, arteritic disorders, diabetic retinopathy, low birth weight, pregnancy complications, severe periodontal disease, psoriasis and insulin dependent diabetes, but is particularly characterized by arteritic lesions. The targeted disruption may be anywhere in the gene, subject only to the requirement that it inhibit production of functional IL-1L1protein. In a preferred embodiment, the disruption removes the entire IL-1L1coding sequence such as that set forth by SEQ ID No. 11. The transgenic animal may be of any species (except human), but is preferably a mammal. In a preferred embodiment, the non-human animal comprising a targeted disruption in the IL-1L1gene, wherein said targeted disruption inhibits production of wild-type IL-1L1 polypeptide so that the phenotype of a non-human mammal homozygous for the targeted disruption is characterized by an altered inflammatory response.

In another aspect, the invention features a cell or cell line, which contains a targeted disruption in the IL-1L1gene. In a preferred embodiment, the cell or cell line is an undifferentiated cell, for example, a stem cell, embryonic stem cell, oocyte or embryonic cell.

Yet in a further aspect, the invention features a method of producing a non-human mammal with a targeted disruption in an interleukin-1 gene. For example, an IL-1L1 knock-out construct can be created with a portion of the IL-1L1gene having an internal portion of said IL-1L1gene replaced by a marker. The knock-out construct can then be transfected into a population of embryonic stem m(ES) cells. Transfected cells can then be selected as expressing the marker. The transfected ES cells can then be introduced into an embryo of an ancestor of said mammal. The embryo can be allowed to develop to term to produce a chimeric mammal with the knock-out construct in its germline. Breeding said chimeric mammal will produce a heterozygous mammal with a targeted disruption in the IL-1L1 gene. Homozygotes can be generated by crossing heterozygotes.

In another aspect, the invention features IL-1L1knock-out constructs, which can be used to generate the animals described above. In one embodiment, the IL-1L1 construct can comprise a portion of the IL-1L1gene, wherein an internal portion of said IL-1L1 gene is replaced by a selectable marker. Preferably, the marker is the neo gene and the portion of the IL-1L1gene is at least 2.5 kb long or 7.0 or 9.5 kb long (including the replaced portion and any IL-1L1flanking sequences). The internal portion preferably covers at least a portion of an exon and in some embodiments it covers all of the exons which encode an IL-1L1 polypeptide.

In still another aspect, the invention features methods for testing agents for effectiveness in treating and/or preventing an inflammatory condition. In one embodiment, the method can employ the transgenic animal or cell lines, as described above. For example, a test agent can be administered to the transgenic animal and the ability of the agent to ameliorate the inflammatory condition can be scored as having effectiveness against said inflammatory condition. Any inflammatory condition with an IL-1 component can be tested using these mammals, but in particular, conditions characterized by arteritic lesions are studied. The method may also be used to test agents that are effective against the IL-1 inflammatory proteins and their downstream components.

The transgenic animals can also be animals containing a transgene, such as reporter gene, under the control of an IL-1L1promoter or fragment thereof. These animals are useful, e.g., for identifying IL-1L1drugs that modulate production of IL-1L1, such as by modulating IL-1L1 gene expression. An IL-1L1 gene promoter can be isolated, e.g., by screening of a genomic library with an IL-1L1cDNA fragment and characterized according to methods known in the art. In a preferred embodiment of the present invention, the transgenic animal containing said IL-1L1reporter gene is used to screen a class of bioactive molecules known as steroid hormones for their ability to modulate IL-1L1 expression. In a more preferred embodiment of the invention, the steroid hormones screened for IL-1L1 expression modulating activity belong to the group known as androgens. In a still more preferred embodiment of the invention, the steroid hormone is testosterone or a testosterone analog. Yet other non-human animals within the scope of the invention include those in which the expression of the endogenous IL-1L1gene has been mutated or “knocked out”. A “knock out” animal is one carrying a homozygous or heterozygous deletion of a particular gene or genes. These animals could be useful to determine whether the absence of IL-1L1 will result in a specific phenotype, in particular whether these mice have or are likely to develop a specific disease, such as high susceptibility to heart disease or cancer. Furthermore these animals are useful in screens for drugs which alleviate or attenuate the disease condition resulting from the mutation of the IL-1L1gene as outlined below. These animals are also useful for determining the effect of a specific amino acid difference, or allelic variation, in an IL-1L1 gene. That is, the IL-1L1 knock out animals can be crossed with transgenic animals expressing, e.g., a mutated form or allelic variant of IL-1L1, thus resulting in an animal which expresses only the mutated protein and not the wild-type IL-1L1protein.

In a preferred embodiment of this aspect of the invention, a transgenic IL-1L1 knock-out mouse, carrying the mutated IL-1L1locus on one or both of its chromosomes, is used as a model system for transgenic or drug treatment of the condition resulting from loss of IL-1L1expression.

Methods for obtaining transgenic and knockout non-human animals are well known in the art. Knock out mice are generated by homologous integration of a “knock out” construct into a mouse embryonic stem cell chromosome which encodes the gene to be knocked out. In one embodiment, gene targeting, which is a method of using homologous recombination to modify an animal's genome, can be used to introduce changes into cultured embryonic stem cells. By targeting a IL-1L1gene of interest in ES cells, these changes can be introduced into the germlines of animals to generate chimeras. The gene targeting procedure is accomplished by introducing into tissue culture cells a DNA targeting construct that includes a segment homologous to a target IL-1L1locus, and which also includes an intended sequence modification to the IL-1L1 genomic sequence (e.g., insertion, deletion, point mutation). The treated cells are then screened for accurate targeting to identify and isolate those which have been properly targeted.

Gene targeting in embryonic stem cells is in fact a scheme contemplated by the present invention as a means for disrupting a IL-1L1 gene function through the use of a targeting transgene construct designed to undergo homologous recombination with one or more IL-1L1genomic sequences. The targeting construct can be arranged so that, upon recombination with an element of a IL-1L1gene, a positive selection marker is inserted into (or replaces) coding sequences of the gene. The inserted sequence functionally disrupts the IL-1L1 gene, while also providing a positive selection trait. Exemplary IL-1L1 targeting constructs are described in more detail below.

Generally, the embryonic stem cells (ES cells) used to produce the knockout animals will be of the same species as the knockout animal to be generated. Thus for example, mouse embryonic stem cells will usually be used for generation of knockout mice.

Embryonic stem cells are generated and maintained using methods well known to the skilled artisan such as those described by Doetschman et al. (1985) J. Embryol. Exp. MoIL-1L1hol. 87:27-45). Any line of ES cells can be used, however, the line chosen is typically selected for the ability of the cells to integrate into and become part of the germ line of a developing embryo so as to create germ line transmission of the knockout construct. Thus, any ES cell line that is believed to have this capability is suitable for use herein. One mouse strain that is typically used for production of ES cells, is the 129J strain. Another ES cell line is murine cell line D3 (American Type Culture Collection, catalog no. CKL 1934) Still another preferred ES cell line is the WW6 cell line (loffe et al. (1995) PNAS 92:7357-7361). The cells are cultured and prepared for knockout construct insertion using methods well known to the skilled artisan, such as those set forth by Robertson in: Teratocarcinomas and Embryonic Stem Cells: A Practical Approach, E. J. Robertson, ed. IRL Press, Washington, D.C. [1987]); by Bradley et al. (1986) Current Topics in Devel. Biol. 20:357-371); and by Hogan et al. (Manipulating the Mouse Embryo: A Laboratory Manual, Cold Spring Harbor Laboratory Press, Cold Spring Harbor, N.Y. [1986]) .

A knock out construct refers to a uniquely configured fragment of nucleic acid which is introduced into a stem cell line and allowed to recombine with the genome at the chromosomal locus of the gene of interest to be mutated. Thus a given knock out construct is specific for a given gene to be targeted for disruption. Nonetheless, many common elements exist among these constructs and these elements are well known in the art. A typical knock out construct contains nucleic acid fragments of not less than about 0.5 kb nor more than about 10.0 kb from both the 5′ and the 3′ ends of the genomic locus which encodes the gene to be mutated. These two fragments are separated by an intervening fragment of nucleic acid which encodes a positive selectable marker, such as the neomycin resistance gene (neo^{R}). The resulting nucleic acid fragment, consisting of a nucleic acid from the extreme 5′ end of the genomic locus linked to a nucleic acid encoding a positive selectable marker which is in turn linked to a nucleic acid from the extreme 3′ end of the genomic locus of interest, omits most of the coding sequence for IL-1L1or other gene of interest to be knocked out. When the resulting construct recombines homologously with the chromosome at this locus, it results in the loss of the omitted coding sequence, otherwise known as the structural gene, from the genomic locus. A stem cell in which such a rare homologous recombination event has taken place can be selected for by virtue of the stable integration into the genome of the nucleic acid of the gene encoding the positive selectable marker and subsequent selection for cells expressing this marker gene in the presence of an appropriate drug (neomycin in this example).

Variations on this basic technique also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targetted gene, resulting in a transgenic animal which expresses a polypeptide of the targetted gene which is defective in one function, while retaining the function of other domains of the encoded polypeptide. This type of “knock-in” mutant frequently has the characteristic of a so-called “dominant negative” mutant because, especially in the case of proteins which homomultimerize, it can specifically block the action of (or “poison”) the polypeptide product of the wild-type gene from which it was derived. In a variation of the knock-in technique, a marker gene is integrated at the genomic locus of interest such that expression of the marker gene comes under the control of the transcriptional regulatory elements of the targeted gene. A marker gene is one that encodes an enzyme whose activity can be detected (e.g., b-galactosidase), the enzyme substrate can be added to the cells under suitable conditions, and the enzymatic activity can be analyzed. One skilled in the art will be familiar with other useful markers and the means for detecting their presence in a given cell. All such markers are contemplated as being included within the scope of the teaching of this invention.

As mentioned above, the homologous recombination of the above described “knock out” and “knock in” constructs is very rare and frequently such a construct inserts nonhomologously into a random region of the genome where it has no effect on the gene which has been targeted for deletion, and where it can potentially recombine so as to disrupt another gene which was otherwise not intended to be altered. Such nonhomologous recombination events can be selected against by modifying the abovementioned knock out and knock in constructs so that they are flanked by negative selectable markers at either end (particularly through the use of two allelic variants of the thymidine kinase gene, the polypeptide product of which can be selected against in expressing cell lines in an appropriate tissue culture medium well known in the art—i.e. one containing a drug such as 5-bromodeoxyuridine). Thus a preferred embodiment of such a knock out or knock in construct of the invention consist of a nucleic acid encoding a negative selectable marker linked to a nucleic acid encoding a 5′ end of a genomic locus linked to a nucleic acid of a positive selectable marker which in turn is linked to a nucleic acid encoding a 3′ end of the same genomic locus which in turn is linked to a second nucleic acid encoding a negative selectable marker Nonhomologous recombination between the resulting knock out construct and the genome will usually result in the stable integration of one or both of these negative selectable marker genes and hence cells which have undergone nonhomologous recombination can be selected against by growth in the appropriate selective media (e.g. media containing a drug such as 5-bromodeoxyuridine for example). Simultaneous selection for the positive selectable marker and against the negative selectable marker will result in a vast enrichment for clones in which the knock out construct has recombined homologously at the locus of the gene intended to be mutated. The presence of the predicted chromosomal alteration at the targeted gene locus in the resulting knock out stem cell line can be confirmed by means of Southern blot analytical techniques which are well known to those familiar in the art. Alternatively, PCR can be used.

Each knockout construct to be inserted into the cell must first be in the linear form. Therefore, if the knockout construct has been inserted into a vector (described infra), linearization is accomplished by digesting the DNA with a suitable restriction endonuclease selected to cut only within the vector sequence and not within the knockout construct sequence.

For insertion, the knockout construct is added to the ES cells under appropriate conditions for the insertion method chosen, as is known to the skilled artisan. For example, if the ES cells are to be electroporated, the ES cells and knockout construct DNA are exposed to an electric pulse using an electroporation machine and following the manufacturer's guidelines for use. After electroporation, the ES cells are typically allowed to recover under suitable incubation conditions. The cells are then screened for the presence of the knock out construct as explained above. Where more than one construct is to be introduced into the ES cell, each knockout construct can be introduced simultaneously or one at a time.

After suitable ES cells containing the knockout construct in the proper location have been identified by the selection techniques outlined above, the cells can be inserted into an embryo. Insertion may be accomplished in a variety of ways known to the skilled artisan, however a preferred method is by microinjection. For microinjection, about 10-30 cells are collected into a micropipet and injected into embryos that are at the proper stage of development to permit integration of the foreign ES cell containing the knockout construct into the developing embryo. For instance, the transformed ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan, and are set forth by, e.g., Bradley et al. (supra).

While any embryo of the right stage of development is suitable for use, preferred embryos are male. In mice, the preferred embryos also have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy (as described above, and in the appended examples) has been employed. In addition, or as an alternative, DNA from tail tissue of the offspring may be screened for the presence of the knockout construct using Southern blots and/or PCR as described above. Offspring that appear to be mosaics may then be crossed to each other, if they are believed to carry the knockout construct in their germ line, in order to generate homozygous knockout animals. Homozygotes may be identified by Southern blotting of equivalent amounts of genomic DNA from mice that are the product of this cross, as well as mice that are known heterozygotes and wild type mice.

Other means of identifying and characterizing the knockout offspring are available. For example, Northern blots can be used to probe the mRNA for the presence or absence of transcripts encoding either the gene knocked out, the marker gene, or both. In addition, Western blots can be used to assess the level of expression of the IL-1L1 gene knocked out in various tissues of the offspring by probing the Western blot with an antibody against the particular IL-1L1protein, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

Yet other methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert target sequences, such that tissue specific and/or temporal control of inactivation of a IL-1L1-gene can be controlled by recombinase sequences (described infra).

Animals containing more than one knockout construct and/or more than one transgene expression construct are prepared in any of several ways. The preferred manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s).

A IL-1L1 transgene can encode the wild-type form of the protein, or can encode homologs thereof, including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of a IL-1L1protein can be essential for many forms of lineage analysis and can additionally provide a means to assess the effects of, for example, lack of IL-1L1expression which might grossly alter development in small patches of tissue within an otherwise normal embryo. Toward this and, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques, which allow for the expression of transgenes can be regulated via site-specific genetic manipulation in vivo, are known to those skilled in the art. For instance, genetic systems are available which allow for the regulated expression of a recombinase that catalyzes the genetic recombination of a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject IL-1L1 proteins. For example, excision of a target sequence which interferes with the expression of a recombinant IL-1L1gene, such as one which encodes an antagonistic homolog or an antisense transcript, can be designed to activate expression of that gene. This interference with expression of the protein can result from a variety of mechanisms, such as spatial separation of the IL-1L1gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

The transgenic animals of the present invention all include within a plurality of their cells a transgene of the present invention, which transgene alters the phenotype of the “host cell” with respect to regulation of cell growth, death and/or differentiation. Since it is possible to produce transgenic organisms of the invention utilizing one or more of the transgene constructs described herein, a general description will be given of the production of transgenic organisms by referring generally to exogenous genetic material. This general description can be adapted by those skilled in the art in order to incorporate specific transgene sequences into organisms utilizing the methods and materials described below.

In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of a recombinant IL-1L1 protein can be regulated via control of recombinase expression.

Use of the cre/loxP recombinase system to regulate expression of a recombinant IL-1L1protein requires the construction of a transgenic animal containing transgenes encoding both the Cre recombinase and the subject protein. Animals containing both the Cre recombinase and a recombinant IL-1L1gene can be provided through the construction of “double” transgenic animals. A convenient method for providing such animals is to mate two transgenic animals each containing a transgene, e.g., a IL-1L1 gene and recombinase gene.

One advantage derived from initially constructing transgenic animals containing a IL-1L1transgene in a recombinase-mediated expressible format derives from the likelihood that the subject protein, whether agonistic or antagonistic, can be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues and/or a desired temporal pattern. Thus, the creation of a founder population in which, for example, an antagonistic IL-1L1transgene is silent will allow the study of progeny from that founder in which disruption of IL-1L1mediated induction in a particular tissue or at certain developmental stages would result in, for example, a lethal phenotype.

Similar conditional transgenes can be provided using prokaryotic promoter sequences which require prokaryotic proteins to be simultaneous expressed in order to facilitate expression of the IL-1L1transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the trans-activating protein, e.g. a recombinase or a prokaryotic protein, is delivered to the tissue and caused to be expressed, such as in a cell-type specific manner. By this method, an IL-1L1A transgene could remain silent into adulthood until “turned on” by the introduction of the trans-activator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2b, H-2d or H-2q haplotypes such as C57BL/6 or DBA/1. The line(s) used to practice this invention may themselves be transgenics, and/or may be knockouts (i.e., obtained from animals which have one or more genes partially or completely suppressed). In one embodiment, the transgene construct is introduced into a single stage embryo. The zygote is the best target for micro-injection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will, in certain embodiments, be a DNA sequence which results in the production of a IL-1L1 protein (either agonistic or antagonistic), and antisense transcript, or a IL-1L1mutant. Further, in such embodiments the sequence will be attached to a transcriptional control element, e.g., a promoter, which preferably allows the expression of the transgene product in a specific type of cell.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

4.7. Screening Assays for IL-1L1 Therapeutics

The invention further provides screening methods for identifying IL-1L1 therapeutics, e.g., for treating and/or preventing the development of diseases or conditions caused by, or contributed to by an abnormal IL-1L1activity or which can benefit from a modulation of an IL-1L1activity or protein level. Examples of such diseases, conditions or disorders such as those involving the inflammatory response inluding without limitation: rheumatoid arthritis, inflammatory bowel disorder, Type I diabetes, psoriasis, osteoporosis, nephropathy in diabetes mellitus, alopecia areata, Graves disease, systemic lupus erythematosus, lichen sclerosis, ulcerative colitis, coronary artery disease, arteritic disorders, diabetic retinopathy, low birth weight, pregnancy complications, severe periodontal disease, psoriasis and insulin dependent diabetes, but is particularly characterized by arteritic lesions, cancer e.g., cancers involving the growth of steroid hormone-responsive tumors (e.g. breast, prostate, or testicular cancer), vascular diseases or disorders (e.g. thrombotic stroke, ischemic stroke, as well as peripheral vascular disease resulting from atherosclerotic and thrombotic processes), cardiac disorders (e.g., myocardial infarction, congestive heart failure, unstable angina and ishemic heart disease); and cardiovascular system diseases and disorders (e.g. those resulting from hypertension, hypotension, cardiomyocyte hypertrophy and congestive heart failure) or other diseases conditions or disorders which result from aberrations or alterations of IL-1L1-dependent processes.

An IL-1L1therapeutic can be any type of compound, including a protein, a peptide, peptidomimetic, small molecule, and nucleic acid. A nucleic acid can be, e.g., a gene, an antisense nucleic acid, a ribozyme, or a triplex molecule. An IL-1L1therapeutic of the invention can be an agonist or an antagonist. Preferred IL-1L1agonists include IL-1L1 proteins or derivatives thereof which mimic at least one IL-1L1 activity, e.g., fibroblast growth factor receptor binding or heparin sulfate binding. Other preferred agonists include compounds which are capable of increasing the production of an IL-1L1 protein in a cell, e.g., compounds capable of up-regulating the expression of an IL-1L1gene, and compounds which are capable of enhancing an IL-1L1activity and/or the interaction of an IL-1L1protein with another molecule, such as a target peptide. Preferred IL-1L1 antagonists include IL-1L1 proteins which are dominant negative proteins, which, e.g., are capable of binding to fibroblast growth factor receptors, but not heparin sulfate. Other preferred antagonists include compounds which decrease or inhibit the production of an IL-1L1protein in a cell and compounds which are capable of downregulating expression of an IL-1L1gene, and compounds which are capable of downregulating an IL-1L1activity and/or interaction of an IL-1L1 protein with another molecule. In another preferred embodiment, an IL-1L1 antagonist is a modified form of a target peptide, which is capable of interacting with the FGFR binding domain of an IL-1L1 protein, but which does not have biological activity, e.g., which is not itself a cell surface receptor.

The invention also provides screening methods for identifying IL-1L1 therapeutics which are capable of binding to an IL-1L1 protein, e.g., a wild-type IL-1L1 protein or a mutated form of an IL-1L1protein, and thereby modulate the growth factor activity of IL-1L1or otherwise cause the degradation of IL-1L1. For example, such an IL-1L1 therapeutic can be an antibody or derivative thereof which interacts specifically with an IL-1L1 protein (either wild-type or mutated).

Thus, the invention provides screening methods for identifying IL-1L1 agonist and antagonist compounds, comprising selecting compounds which are capable of interacting with an IL-1L1 protein or with a molecule capable of interacting with an IL-1L1 protein such as an FGF receptor and/or heparin sulfate and/or a compound which is capable of modulating the interaction of an IL-1L1 protein with another molecule, such as a receptor and/or heparin sulfate. In general, a molecule which is capable of interacting with an IL-1L1protein is referred to herein as “IL-1L1 binding partner”.

The compounds of the invention can be identified using various assays depending on the type of compound and activity of the compound that is desired. In addition, as described herein, the test compounds can be further tested in animal models. Set forth below are at least some assays that can be used for identifying IL-1L1 therapeutics. It is within the skill of the art to design additional assays for identifying IL-1L1therapeutics.

4.7.1. Cell-Free Assays

Cell-free assays can be used to identify compounds which are capable of interacting with an IL-1L1protein or binding partner, to thereby modify the activity of the IL-1L1protein or binding partner. Such a compound can, e.g., modify the structure of an IL-1L1 protein or binding partner and thereby effect its activity. Cell-free assays can also be used to identify compounds which modulate the interaction between an IL-1L1protein and an IL-1L1binding partner, such as a target peptide. In a preferred embodiment, cell-free assays for identifying such compounds consist essentially in a reaction mixture containing an IL-1L1 protein and a test compound or a library of test compounds in the presence or absence of a binding partner. A test compound can be, e.g., a derivative of an IL-1L1 binding partner, e.g., a biologically inactive target peptide, or a small molecule.

Accordingly, one exemplary screening assay of the present invention includes the steps of contacting an IL-1L1protein or functional fragment thereof or an IL-1L1binding partner with a test compound or library of test compounds and detecting the formation of complexes. For detection purposes, the molecule can be labeled with a specific marker and the test compound or library of test compounds labeled with a different marker. Interaction of a test compound with an IL-1L1 protein or fragment thereof or IL-1L1 binding partner can then be detected by determining the level of the two labels after an incubation step and a washing step. The presence of two labels after the washing step is indicative of an interaction.

An interaction between molecules can also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds can be immobilized on a sensor surface, e.g., which forms one wall of a micro-flow cell. A solution containing the IL-1L1 protein, functional fragment thereof, IL-1L1 analog or IL-1L1 binding partner is then flown continuously over the sensor surface. A change in the resonance angle as shown on a signal recording, indicates that an interaction has occurred. This technique is further described, e.g., in BIAtechnology Handbook by Pharmacia.

Another exemplary screening assay of the present invention includes the steps of (a) forming a reaction mixture including: (i) an IL-1L1 polypeptide, (ii) an IL-1L1binding partner, and (iii) a test compound; and (b) detecting interaction of the IL-1L1 and the IL-1 L1 binding protein. The IL-1L1polypeptide and IL-1L1binding partner can be produced recombinantly, purified from a source, e.g., plasma, or chemically synthesized, as described herein. A statistically significant change (potentiation or inhibition) in the interaction of the IL-1L1and IL-1L1binding protein in the presence of the test compound, relative to the interaction in the absence of the test compound, indicates a potential agonist (mimetic or potentiator) or antagonist (inhibitor) of IL-1L1bioactivity for the test compound. The compounds of this assay can be contacted simultaneously. Alternatively, an IL-1L1protein can first be contacted with a test compound for an appropriate amount of time, following which the IL-1L1 binding partner is added to the reaction mixture. The efficacy of the compound can be assessed by generating dose response curves from data obtained using various concentrations of the test compound. Moreover, a control assay can also be performed to provide a baseline for comparison. In the control assay, isolated and purified IL-1L1 polypeptide or binding partner is added to a composition containing the IL-1L1 binding partner or IL-1L1polypeptide, and the formation of a complex is quantitated in the absence of the test compound.

Complex formation between an IL-1L1protein and an IL-1L1binding partner may be detected by a variety of techniques. Modulation of the formation of complexes can be quantitated using, for example, detectably labeled proteins such as radiolabeled, fluorescently labeled, or enzymatically labeled IL-1L1proteins or IL-1L1binding partners, by immunoassay, or by chromatographic detection.

Typically, it will be desirable to immobilize either IL-1L1or its binding partner to facilitate separation of complexes from uncomplexed forms of one or both of the proteins, as well as to accommodate automation of the assay. Binding of IL-1L1to an IL-1L1 binding partner, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/IL-1L1 (GST/IL-1L1) fusion proteins can be adsorbed onto glutathione sepharose beads (Sigma Chemical, St. Louis, Mo.) or glutathione derivatized microtitre plates, which are then combined with the IL-1L1 binding partner, e.g. an 35S-labeled IL-1L1binding partner, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of IL-1L1protein or IL-1L1binding partner found in the bead fraction quantitated from the gel using standard electrophoretic techniques such as described in the appended examples.

Other techniques for immobilizing proteins on matrices are also available for use in the subject assay. For instance, either IL-1L1or its cognate binding partner can be immobilized utilizing conjugation of biotin and streptavidin. For instance, biotinylated IL-1L1 molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with IL-1L1 can be derivatized to the wells of the plate, and IL-1L1trapped in the wells by antibody conjugation. As above, preparations of an IL-1L1 binding protein and a test compound are incubated in the IL-1L1presenting wells of the plate, and the amount of complex trapped in the well can be quantitated. Exemplary methods for detecting such complexes, in addition to those described above for the GST-immobilized complexes, include immunodetection of complexes using antibodies reactive with the IL-1L1 binding partner, or which are reactive with IL-1L1 protein and compete with the binding partner; as well as enzyme-linked assays which rely on detecting an enzymatic activity associated with the binding partner, either intrinsic or extrinsic activity. In the instance of the latter, the enzyme can be chemically conjugated or provided as a fusion protein with the IL-1L1binding partner. To illustrate, the IL-1L1binding partner can be chemically cross-linked or genetically fused with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating one of the proteins trapped in the complex, antibodies against the protein, such as anti-IL-1L1 antibodies, can be used. Alternatively, the protein to be detected in the complex can be “epitope tagged” in the form of a fusion protein which includes, in addition to the IL-1L1 sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharmacia, N.J.).

Cell-free assays can also be used to identify compounds which interact with an IL-1L1protein and modulate an activity of an IL-1L1protein. Accordingly, in one embodiment, an IL-1L1protein is contacted with a test compound and the catalytic activity of IL-1L1is monitored. In one embodiment, the abililty of IL-1L1to bind a target molecule is determined. The binding affinity of IL-1L1 to a target molecule can be determined according to methods known in the art.

4.7.2. Cell Based Assays

In addition to cell-free assays, such as described above, IL-1L1 proteins as provided by the present invention, facilitate the generation of cell-based assays, e.g., for identifying small molecule agonists or antagonists. In one embodiment, a cell expressing an IL-1L1receptor protein on the outer surface of its cellular membrane is incubated in the presence of a test compound alone or in the presence of a test compound and an IL-1L1 protein and the interaction between the test compound and the IL-1L1receptor protein or between the IL-1L1 protein (preferably a tagged IL-1L1 protein) and the IL-1L1 receptor is detected, e.g., by using a microphysiometer (McConnell et al. (1992) Science 257:1906). An interaction between the IL-1L1receptor protein and either the test compound or the IL-1L1 protein is detected by the microphysiometer as a change in the acidification of the medium. This assay system thus provides a means of identifying molecular antagonists which, for example, function by interfering with IL-1L1-IL-1L1 receptor interactions, as well as molecular agonist which, for example, function by activating an IL-1L1 receptor.

Cell based assays can also be used to identify compounds which modulate expression of an IL-1L1gene, modulate translation of an IL-1L1mRNA, or which modulate the stability of an IL-1L1 mRNA or protein. Accordingly, in one embodiment, a cell which is capable of producing IL-1L1, e.g., a choriocarcinoma cell line such as JEG-3, is incubated with a test compound and the amount of IL-1L1 produced in the cell medium is measured and compared to that produced from a cell which has not been contacted with the test compound. The specificity of the compound vis a vis IL-1L1 can be confirmed by various control analysis, e.g., measuring the expression of one or more control genes. Compounds which can be tested include small molecules, proteins, and nucleic acids. In particular, this assay can be used to determine the efficacy of IL-1L1 antisense molecules or ribozymes.

In another embodiment, the effect of a test compound on transcription of an IL-1L1gene is determined by transfection experiments using a reporter gene operatively linked to at least a portion of the promoter of an IL-1L1gene. A promoter region of a gene can be isolated, e.g., from a genomic library according to methods known in the art. The reporter gene can be any gene encoding a protein which is readily quantifiable, e.g, the luciferase or CAT gene. Such reporter gene are well known in the art.

In preferred embodiments, the invention provides cell-based assays employing the choriocarcinoma cell line JEG-3. Analysis of this cell line has shown that it produces a 17 kDa IL-1L1polypeptide which is immunoprecipitated with rabbit anti-IL-1L1 polyclonal antiserum. Accordingly, this cell line can be adapted for screening assays for agents which up-regulate or down-regulate the expression of the IL-1L1gene or otherwise affect the steady-state level of an IL-1L1polypeptide(s) or the efficiency of an IL-1L1 polypeptide post-translational activity such as an IL-1L1 proteolytic processing event, IL-1L1 glycosylation, IL-1L1 phosphorylation or IL-1L1 secretion.

In other preferred embodiments, assays for interleukin-1 agonist and antagonist activities are employed. For example, human skin fibroblasts express interleukin-6 in response to IL-1-like activators such as IL-1a and IL-1b. Accordingly, this cell line can be employed in assays for IL-1 agonist activities. Furthermore, the same cell line provides an assay for IL-1 antagonists by screening for compounds which inhibit or reduce an IL-1a or IL-1b-dependent activation of the interleukin-6 gene. Still further, this cell line can be employed in conjunction with the IL-1 receptor antagonist polypeptide to identify IL-1L1 polypeptides and IL-1L1 polypeptide agonists and antagonists which affect an IL-1 receptor antagonist activity, such as the ability of the IL-1ra to block IL-1b -dependent activation of the interleukin-6 gene.

Other cell-based screening assays which can be employed in the method of the present invention are known or would be apparent to one of skill in the art. For example, IL-1 agonists and antagonists may be identified by their ability to affect downstream IL-1 dependent activation of the NF-kB (Reddy et al. (1997) Mol Cell Biol 19: 4798-805). IL-1 dependent activation occurs by multiple mechanisms, including the activation of IkappaB kinases, particularly the beta subunit (Li et al. (1999) J Exp Med. 189: 1839-45) which leads to phosphorylation, and subsequent proteolytic degradation by the proteosome, of the IkB inhibitor of NF-kB which otherwise inhibits nuclear localization of the to NF-kB transcriptional activator. IL-1 also effects activation of NF-kB through activation of phosphatidylinositol 3-kinase which transduces a signal resulting in phosphorylation of the p65/RelA subunit thereby effecting NF-kB-dependent transcriptional activation of downstream genes (see e.g. Sizemore, et al. (1999) Mol Cell Biol 19: 4798-805). Accordingly IL-1 inducible inflammatory response gene reporters may be used to assay IL-1 agonist and antagonist activities by the method of the present invention.

In addition, IL-1b is known to induce the nitric oxide synthase (iNos) and cyclooxygenase-2 genes in cardiac myocytes (LaPointe et al. (1999) Hypertension 33: 276-82). Furthermore, of particular significance in light of the expression of the IL-1L1gene in human placenta are the numerous genes induced in human umbilical arterial endothelial cells (HUAEC) by interleukin-1 (Ko et al. (1 999) Mol Cell Probes 13: 203-11). These include the basic fibroblast growth factor (bFGF) and vascular endothelial growth factor (VEGF) genes, which regulate angioneogenesis and affect the development of atherosclerosis, as well as the granulocyte-colony stimulating factor (G-CSF), granulocyte macrophage-colony stimulating factor (GM-CSF) and stem cell factor (SCF) genes which are important growth factor effectors of haematopoiesis. Still other affects of IL-1 on the activation or repression of specific genes may be exploited for the development of cell based assays for IL-1L1and IL-1L1 agonist and antagonist activities (see e.g Rohn et al. (1999) J Immunol 162: 886-96 (IL-1 repression of class II MHC expression); Pellacani et al. (1999) J Biol Chem 274: 1525-32 (IL-1 activation of nonhistone chromosomal high mobility group (HMG) gene expression); and Borghaei et al. (1998) Biochem Biophys Res Commun 251: 334-8 (IL-1 induction of mitogen-inducible nuclear orphan receptor (MINoR) gene expression).

This invention further pertains to novel agents identified by the above-described screening assays and uses thereof for treatments as described herein.

4.8. Predictive Medicine

The invention further features predictive medicines, which are based, at least in part, on the identity of the novel IL-1L1genes and alterations in the genes and related pathway genes, which affect the expression level and/or function of the encoded IL-1L1 protein in a subject.

For example, information obtained using the diagnostic assays described herein (alone or in conjunction with information on another genetic defect, which contributes to the same disease) is useful for diagnosing or confirming that a symptomatic subject (e.g. a subject symptomatic for inflammatory rheumatoid arthritis), has a genetic defect (e.g. in an IL-1L1gene or in a gene that regulates the expression of an IL-1L1gene), which causes or contributes to the particular disease or disorder. Alternatively, the information (alone or in conjunction with information on another genetic defect, which contributes to the same disease) can be used prognostically for predicting whether a non-symptomatic subject is likely to develop a disease or condition, which is caused by or contributed to by an abnormal IL-1L1 activity or protein level in a subject. Based on the prognostic information, a doctor can recommend a regimen (e.g. diet or exercise) or therapeutic protocol, useful for preventing or prolonging onset of the particular disease or condition in the individual.

In addition, knowledge of the particular alteration or alterations, resulting in defective or deficient IL-1L1genes or proteins in an individual (the IL-1L1genetic profile), alone or in conjunction with information on other genetic defects contributing to the same disease (the genetic profile of the particular disease) allows customization of therapy for a particular disease to the individual's genetic profile, the goal of “pharmacogenomics”. For example, an individual's IL-1L1 genetic profile or the genetic profile of a disease or condition, to which IL-1L1 genetic alterations cause or contribute, can enable a doctor to 1) more effectively prescribe a drug that will address the molecular basis of the disease or condition; and 2) better determine the appropriate dosage of a particular drug. For example, the expression level of IL-1L1proteins, alone or in conjunction with the expression level of other genes, known to contribute to the same disease, can be measured in many patients at various stages of the disease to generate a transcriptional or expression profile of the disease. Expression patterns of individual patients can then be compared to the expression profile of the disease to determine the appropriate drug and dose to administer to the patient.

The ability to target populations expected to show the highest clinical benefit, based on the IL-1L1or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since the use of IL-1L1as a marker is useful for optimizing effective dose).

These and other methods are described in further detail in the following sections.

4.8.1. Prognostic and Diagnostic Assays

The present methods provide means for determining if a subject has (diagnostic) or is at risk of developing (prognostic) a disease, condition or disorder that is associated with an aberrant IL-1L1activity, e.g., an aberrant level of IL-1L1protein or an aberrant IL-1L1 bioactivity. Examples of such diseases, conditions or disorders include without limitation: inflammatory diseases including rheumatoid arthritis, inflammatory bowel disorder, psoriasis, lupus erythematosus, ulcerative colitis and alopecia areata as well as diabetic nephropathy; cancers involving the growth factor cytokines or steroid hormone-responsive tumors (e.g. breast, prostate, or testicular cancer); vascular diseases or disorders (e.g. thrombotic stroke, ischemic stroke, as well as peripheral vascular disease resulting from atherosclerotic and thrombotic processes); cardiac disorders (e.g. myocardial infarction, unstable angina and ishemic heart disease); cardiovascular system diseases and disorders (e.g. those resulting from hypertension, hypotension, cardiomyocyte hypertrophy and congestive heart failure) wound healing; limb regeneration; neurological damage or disease (e.g. that associated with Alzheimer's disease, Parkinson's disease, AIDS-related complex, or cerebral palsy); or other diseases conditions or disorders which result from aberrations or alterations of IL-1L1-dependent processes including: collateral growth and remodeling of cardiac blood vessels, angiogenesis, cellular transformation through autocrine or paracrine mechanisms, chemotactic stimulation of cells (e.g. endothelial), neurite outgrowth of neuronal precursor cell types (e.g. PC12 phaeochromoctoma), maintenance of neural physiology of mature neurons, proliferation of embryonic mesenchyme and limb-bud precursor tissue, mesoderm induction and other developmental processes, stimulation of collagenase and plasminogen activator secretion, tumor vascularization, as well as tumor invasion and metastasis.

Accordingly, the invention provides methods for determining whether a subject has or is likely to develop, a disease or condition that is caused by or contributed to by an abnormal IL-1L1 level or bioactivity, for example, comprising determining the level of an IL-1L1 gene or protein, an IL-1L1 bioactivity and/or the presence of a mutation or particular polymorphic variant in the IL-1L1 gene.

In one embodiment, the method comprises determining whether a subject has an abnormal mRNA and/or protein level of IL-1L1, such as by Northern blot analysis, reverse transcription-polymerase chain reaction (RT-PCR), in situ hybridization, immunoprecipitation, Western blot hybridization, or immunohistochemistry. According to the method, cells are obtained from a subject and the IL-1L1protein or mRNA level is determined and compared to the level of IL-1L1protein or mRNA level in a healthy subject. An abnormal level of IL-1L1 polypeptide or mRNA level is likely to be indicative of an aberrant IL-1L1 activity.

In another embodiment, the method comprises measuring at least one activity of IL-1L1. For example, the affinity of IL-1L1 for heparin, can be determined, e.g., as described herein. Similarly, the constant of affinity of an IL-1L1protein of a subject with a binding partner (e.g. an IL-1 type I or type II receptor) can be determined. Comparison of the results obtained with results from similar analysis performed on IL-1L1proteins from healthy subjects is indicative of whether a subject has an abnormal IL-1L1 activity.

In preferred embodiments, the methods for determining whether a subject has or is at risk for developing a disease, which is caused by or contributed to by an aberrant IL-1L1 activity is characterized as comprising detecting, in a sample of cells from the subject, the presence or absence of a genetic alteration characterized by at least one of (i) an alteration affecting the integrity of a gene encoding an IL-1L1 polypeptide, or (ii) the mis-expression of the IL-1L1gene. For example, such genetic alterations can be detected by ascertaining the existence of at least one of (i) a deletion of one or more nucleotides from an IL-1L1 gene, (ii) an addition of one or more nucleotides to an IL-1L1 gene, (iii) a substitution of one or more nucleotides of an IL-1L1gene, (iv) a gross chromosomal rearrangement of an IL-1L1gene, (v) a gross alteration in the level of a messenger RNA transcript of an IL-1L1 gene, (vii) aberrant modification of an IL-1L1gene, such as of the methylation pattern of the genomic DNA, (vii) the presence of a non-wild type splicing pattern of a messenger RNA transcript of an IL-1L1gene, (viii) a non-wild type level of an IL-1L1polypeptide, (ix) allelic loss of an IL-1L1gene, and/or (x) inappropriate post-translational modification of an IL-1L1 polypeptide. As set out below, the present invention provides a large number of assay techniques for detecting alterations in an IL-1L1 gene. These methods include, but are not limited to, methods involving sequence analysis, Southern blot hybridization, restriction enzyme site mapping, and methods involving detection of absence of nucleotide pairing between the nucleic acid to be analyzed and a probe. These and other methods are further described infra.

Specific diseases or disorders, e.g., genetic diseases or disorders, are associated with specific allelic variants of polymorphic regions of certain genes, which do not necessarily encode a mutated protein. Thus, the presence of a specific allelic variant of a polymorphic region of a gene, such as a single nucleotide polymorphism (“SNP”), in a subject can render the subject susceptible to developing a specific disease or disorder. Polymorphic regions in genes, e.g, IL-1L1 genes, can be identified, by determining the nucleotide sequence of genes in populations of individuals. If a polymorphic region, e.g., SNP is identified, then the link with a specific disease can be determined by studying specific populations of individuals, e.g, individuals which developed a specific disease, such as congestive heart failure, hypertension, hypotension, or a cancer (e.g. a cancer involving growth of a steroid responsive tumor or tumors). A polymorphic region can be located in any region of a gene, e.g., exons, in coding or non coding regions of exons, introns, and promoter region.

It is likely that IL-1L1genes comprise polymorphic regions, specific alleles of which may be associated with specific diseases or conditions or with an increased likelihood of developing such diseases or conditions. Thus, the invention provides methods for determining the identity of the allele or allelic variant of a polymorphic region of an IL-1L1 gene in a subject, to thereby determine whether the subject has or is at risk of developing a disease or disorder associated with a specific allelic variant of a polymorphic region.

In an exemplary embodiment, there is provided a nucleic acid composition comprising a nucleic acid probe including a region of nucleotide sequence which is capable of hybridizing to a sense or antisense sequence of an IL-1L1gene or naturally occurring mutants thereof, or 5′ or 3′ flanking sequences or intronic sequences naturally associated with the subject IL-1L1genes or naturally occurring mutants thereof. The nucleic acid of a cell is rendered accessible for hybridization, the probe is contacted with the nucleic acid of the sample, and the hybridization of the probe to the sample nucleic acid is detected. Such techniques can be used to detect alterations or allelic variants at either the genomic or mRNA level, including deletions, substitutions, etc., as well as to determine mRNA transcript levels.

A preferred detection method is allele specific hybridization using probes overlapping the mutation or polymorphic site and having about 5, 10, 20, 25, or 30 nucleotides around the mutation or polymorphic region. In a preferred embodiment of the invention, several probes capable of hybridizing specifically to allelic variants, such as single nucleotide polymorphisms, are attached to a solid phase support, e.g., a “chip”. Oligonucleotides can be bound to a solid support by a variety of processes, including lithography. For example a chip can hold up to 250,000 oligonucleotides. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

In certain embodiments, detection of the alteration comprises utilizing the probe/primer in a polymerase chain reaction (PCR) (see, e.g. U.S. Pat. Nos. 4,683,195 and 4,683,202), such as anchor PCR or RACE PCR, or, alternatively, in a ligase chain reaction (LCR) (see, e.g., Landegran et al. (1988) Science 241:1077-1080; and Nakazawa et al. (1994) PNAS 91:360-364), the latter of which can be particularly useful for detecting point mutations in the IL-1L1 gene (see Abravaya et al. (1995) Nuc Acid Res 23:675-682). In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize to an IL-1L1gene under conditions such that hybridization and amplification of the IL-1L1 gene (if present) occurs, and (iv) detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al., 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In a preferred embodiment of the subject assay, mutations in, or allelic variants, of an IL-1L1 gene from a sample cell are identified by alterations in restriction enzyme cleavage patterns. For example, sample and control DNA is isolated, amplified (optionally), digested with one or more restriction endonucleases, and fragment length sizes are determined by gel electrophoresis. Moreover, the use of sequence specific ribozymes (see, for example, U.S. Pat. No. 5,498,531) can be used to score for the presence of specific mutations by development or loss of a ribozyme cleavage site.

In yet another embodiment, any of a variety of sequencing reactions known in the art can be used to directly sequence the IL-1L1gene and detect mutations by comparing the sequence of the sample IL-1L1 with the corresponding wild-type (control) sequence. Exemplary sequencing reactions include those based on techniques developed by Maxim and Gilbert (Proc. Natl Acad Sci USA (1977) 74:560) or Sanger (Sanger et al (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labelled) RNA or DNA containing the wild-type IL-1L1 sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in IL-1L1 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an IL-1L1 sequence, e.g., a wild-type IL-1L1 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations or the identity of the allelic variant of a polymorphic region in IL-1L1 genes. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control IL-1L1nucleic acids are denatured and allowed to renature. The secondary structure of single-stranded nucleic acids varies according to sequence, the resulting alteration in electrophoretic mobility enables the detection of even a single base change. The DNA fragments may be labelled or detected with labelled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting point mutations or the identity of the allelic variant of a polymorphic region include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238. In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al., Science 241:1077-1080 (1988). The OLA protocol uses two oligonucleotides which are designed to be capable of hybridizing to abutting sequences of a single strand of a target. One of the oligonucleotides is linked to a separation marker, e.g,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA, which is then detected using OLA.

Several techniques based on this OLA method have been developed and can be used to detect specific allelic variants of a polymorphic region of an IL-1L1gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide polymorphisms in an IL-1L1 gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A. -C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. -C., et al., Amer.J. Hum. Genet. 52:46-59 (1993)).

For mutations that produce premature termination of protein translation, the protein truncation test (PTT) offers an efficient diagnostic approach (Roest, et. al., (1993) Hum. Mol. Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

The methods described herein may be performed, for example, by utilizing pre-packaged diagnostic kits comprising at least one probe nucleic acid, primer set; and/or antibody reagent described herein, which may be conveniently used, e.g., in clinical settings to diagnose patients exhibiting symptoms or family history of a disease or illness involving an IL-1L1polypeptide.

Any cell type or tissue may be utilized in the diagnostics described below. In a preferred embodiment a bodily fluid, e.g., blood, is obtained from the subject to determine the presence of a mutation or the identity of the allelic variant of a polymorphic region of an IL-1L1gene. A bodily fluid, e.g, blood, can be obtained by known techniques (e.g. venipuncture). Alternatively, nucleic acid tests can be performed on dry samples (e.g. hair or skin). For prenatal diagnosis, fetal nucleic acid samples can be obtained from maternal blood as described in International Patent Application No. WO91/07660 to Bianchi. Alternatively, amniocytes or chorionic villi may be obtained for performing prenatal testing.

When using RNA or protein to determine the presence of a mutation or of a specific allelic variant of a polymorphic region of an IL-1L1gene, the cells or tissues that may be utilized must express the IL-1L1 gene. Preferred cells for use in these methods include cardiac cells (see Examples). Alternative cells or tissues that can be used, can be identified by determining the expression pattern of the specific IL-1L1gene in a subject, such as by Northern blot analysis.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

Antibodies directed against wild type or mutant IL-1L1polypeptides or allelic variants thereof, which are discussed above, may also be used in disease diagnostics and prognostics. Such diagnostic methods, may be used to detect abnormalities in the level of IL-1L1 polypeptide expression, or abnormalities in the structure and/or tissue, cellular, or subcellular location of an IL-1L1 polypeptide. Structural differences may include, for example, differences in the size, electronegativity, or antigenicity of the mutant IL-1L1 polypeptide relative to the normal IL-1L1polypeptide. Protein from the tissue or cell type to be analyzed may easily be detected or isolated using techniques which are well known to one of skill in the art, including but not limited to western blot analysis. For a detailed explanation of methods for carrying out Western blot analysis, see Sambrook et al, 1989, supra, at Chapter 18. The protein detection and isolation methods employed herein may also be such as those described in Harlow and Lane, for example, (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of IL-1L1polypeptides. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the IL-1L1 polypeptide, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labeling an anti-IL-1L1 polypeptide specific antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as 152 Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Moreover, it will be understood that any of the above methods for detecting alterations in a gene or gene product or polymorphic variants can be used to monitor the course of treatment or therapy.

4.8.2. Pharmacogenomics

Knowledge of the particular alteration or alterations, resulting in defective or deficient IL-1L1 genes or proteins in an individual (the IL-1L1 genetic profile), alone or in conjunction with information on other genetic defects contributing to the same disease (the genetic profile of the particular disease) allows a customization of the therapy for a particular disease to the individual's genetic profile, the goal of “pharmacogenomics”. For example, subjects having a specific allele of an IL-1L1gene may or may not exhibit symptoms of a particular disease or be predisposed of developing symptoms of a particular disease. Further, if those subjects are symptomatic, they may or may not respond to a certain drug, e.g., a specific IL-1L1 therapeutic, but may respond to another. Thus, generation of an IL-1L1 genetic profile, (e.g., categorization of alterations in IL-1L1genes which are associated with the development of a particular disease), from a population of subjects, who are symptomatic for a disease or condition that is caused by or contributed to by a defective and/or deficient IL-1L1gene and/or protein (an IL-1L1genetic population profile) and comparison of an individual's IL-1L1 profile to the population profile, permits the selection or design of drugs that are expected to be safe and efficacious for a particular patient or patient population (i.e., a group of patients having the same genetic alteration).

For example, an IL-1L1 population profile can be performed, by determining the IL-1L1 profile, e.g., the identity of IL-1L1 genes, in a patient population having a disease, which is caused by or contributed to by a defective or deficient IL-1L1 gene. Optionally, the IL-1L1population profile can further include information relating to the response of the population to an IL-1L1 therapeutic, using any of a variety of methods, including, monitoring: 1) the severity of symptoms associated with the IL-1L1 related disease, 2) IL-1L1 gene expression level, 3) IL-1L1 mRNA level, and/or 4) IL-1L1 protein level and (iii) dividing or categorizing the population based on the particular genetic alteration or alterations present in its IL-1L1gene or an IL-1L1pathway gene. The IL-1L1genetic population profile can also, optionally, indicate those particular alterations in which the patient was either responsive or non-responsive to a particular therapeutic. This information or population profile, is then useful for predicting which individuals should respond to particular drugs, based on their individual IL-1L1 profile.

In a preferred embodiment, the IL-1L1 profile is a transcriptional or expression level profile and step (i) is comprised of determining the expression level of IL-1L1 proteins, alone or in conjunction with the expression level of other genes, known to contribute to the same disease. The IL-1L1 profile can be measured in many patients at various stages of the disease.

Pharmacogenomic studies can also be performed using transgenic animals. For example, one can produce transgenic mice, e.g., as described herein, which contain a specific allelic variant of an IL-1L1gene. These mice can be created, e.g, by replacing their wild-type IL-1L1gene with an allele of the human IL-1L1 gene. The response of these mice to specific IL-1L1 therapeutics can then be determined.

4.8.3. Monitoring of Effects of IL-1L1Therapeutics During Clinical Trials

The ability to target populations expected to show the highest clinical benefit, based on the IL-1L1or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since the use of IL-1L1as a marker is useful for optimizing effective dose).

The treatment of an individual with an IL-1L1therapeutic can be monitored by determining IL-1L1 characteristics, such as IL-1L1 protein level or activity, IL-1L1 mRNA level, and/or IL-1L1transcriptional level. This measurements will indicate whether the treatment is effective or whether it should be adjusted or optimized. Thus, IL-1L1 can be used as a marker for the efficacy of a drug during clinical trials.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a preadministration sample from a subject prior to administration of the agent; (ii) detecting the level of expression of an IL-1L1 protein, mRNA, or genomic DNA in the preadministration sample; (iii) obtaining one or more post-administration samples from the subject; (iv) detecting the level of expression or activity of the IL-1L1protein, mRNA, or genomic DNA in the post-administration samples; (v) comparing the level of expression or activity of the IL-1L1 protein, mRNA, or genomic DNA in the preadministration sample with the IL-1L1 protein, mRNA, or genomic DNA in the post administration sample or samples; and (vi) altering the administration of the agent to the subject accordingly. For example, increased administration of the agent may be desirable to increase the expression or activity of IL-1L1 to higher levels than detected, i.e., to increase the effectiveness of the agent. Alternatively, decreased administration of the agent may be desirable to decrease expression or activity of IL-1L1to lower levels than detected, i.e., to decrease the effectiveness of the agent.

Cells of a subject may also be obtained before and after administration of an IL-1L1 therapeutic to detect the level of expression of genes other than IL-1L1, to verify that the IL-1L1 therapeutic does not increase or decrease the expression of genes which could be deleterious. This can be done, e.g., by using the method of transcriptional profiling. Thus, mRNA from cells exposed in vivo to an IL-1L1 therapeutic and mRNA from the same type of cells that were not exposed to the IL-1L1 therapeutic could be reverse transcribed and hybridized to a chip containing DNA from numerous genes, to thereby compare the expression of genes in cells treated and not treated with an IL-1L1-therapeutic. If, for example an IL-1L1therapeutic turns on the expression of a proto-oncogene in an individual, use of this particular IL-1L1 therapeutic may be undesirable.

4.9. IL-1L1 Therapeutics and Methods of Treatment

The present invention provides for both prophylactic and therapeutic methods of treating a subject having or likely to develop a disorder associated with aberrant interleukin-1 expression or activity, e.g., inflammation or autoimmune disorders. The cytokines interleukin-1 (IL-1) and tumour necrosis factor (TNF) are important mediators of inflammatory responses, and appear to play a central role in the pathogenesis of many chronic inflammatory diseases. It is now well documented that their biological activities in vivo are sufficient to reproduce local inflammation and matrix catabolism by attracting and activating white blood cells to tissues, and stimulating their secretion of other lymphocytotropic cytokines and catabolic enzymes. Higher production of these cytokines have also been associated with response to infection, where local induction of IL-1 and TNF facilitates the elimination of the microbial invasion. Classic studies however also report that in some infectious conditions very high levels of monocytic cytokines are produced, which activate a cascade of concomitant events such as tissue catabolism, vascular reactivity and hyper-coagulation with damaging effects on the host.

For example, cytokines function throughout development and may be of particular importance in the development and function of the human placenta (reviewed in Jokhi et al. (1997) Cytokine 9: 126-37). A variety of cytokines have been demonstrated at the placental-uterine interface, but the exact cellular sources of production have not yet been identified due to the complex tissue topography of the implantation site. The expression of the cytokines EGF, interleukin 1 beta (IL-1 beta), IL-2, IL-3, interferon alpha (IFN-alpha), IFN-gamma, tumour necrosis factor alpha (TNF-alpha) and transforming growth factor beta I (TGF-beta 1) have been assayed from cells isolated from the placenta and decidua. Furthermore, the expression of the cytokine receptors IGF-1r, PDGF-r alpha/beta, IL-1rII, IL-6r, IL-7r, IFN-gamma r, TNF-rp80 and endoglin by placental and uterine cells has been assessed by both immunohistological and flow cytometric methods. These studies reveal a complex array of cytokine activities at the human placental-uterine interface.

The pro-inflammatory cytokines IL-1, IL-6 and tumor necrosis factor-alpha (TNFa) appear to function in the link between prenatal intrauterine infection (IUI) and neonatal brain damage. Furthermore, maternal IUI increases the risk of preterm delivery, which in turn is associated with an increased risk of intraventricular hemorrhage, neonatal white matter damage, and subsequent cerebal palsy (Dammann et al. (1997) Pediatr Res 42: 1-8). IL-1, IL-6, and TNFa have been found associated with IUI, preterm birth, neonatal infections, and neonatal brain damage. The presence of such cytokines in the three relevant maternal/fetal compartments (uterus, fetal circulation, and fetal brain) and their potential ability of the cytokines to cross boundaries (both placental and the blood-brain barrier) between these compartments suggests their potential role in intraventricular hemorrhage, neonatal white matter damage during prenatal maternal infection. Therefore interrupting the proinflammatory cytokine cascade mediated by IL-1 might prevent later disability in those born near the end of the second trimester.

Interleukin-1 beta (IL-1 β) is present in normal amniotic fluid and is produced by human placental macrophages. The amount of IL-1 β detected in the second trimester amniotic fluid has been shown to exhibit a threefold increase with the onset of labor. IL-1 β is a potent stimulator of the synthesis of prostaglandins by decidua and by amnion. High levels of the prostaglandins PGE₂ and PGF 2α in the amniotic fluid have been associated with preterm labor and intraamniotic infection. This may be explained by the fact that amnion from women with preterm labor and histologic chorioamnionitis produced more PGE₂ than amnion from women without placental inflammation. Such elevated levels of PGE₂ have been associated with premature low birth weight (PBLW) even in the absence of clinical or subclinical genitourinary tract infection and indeed the majority of PLBW deliveries may be caused by an infection of unknown origin. IL-1 was the first cytokine implicated in the onset of labor in the presence of infection. IL-1 is produced in vitro by human decidua in response to bacterial products. In patients with preterm labor and bacteria in the amniotic cavity, amniotic fluid IL-1 bioactivity and concentrations are elevated. Placental necrosis and fetal resorption can be induced in rats by the injection of recombinant human IL-1 β on day 12 of gestation. Furthermore, both the amniotic fluid IL-1 β concentration and bioactivity are elevated during labor compared to controls. In addition, IL-1 β is known to stimulate prostaglandin production by amnion and decidua in vitro.

Accordingly, IL-1L1 therapeutics of the present invention include those which antagonize interleukin-1 dependent disorders of the human placental including intraventricular hemorrhage, neonatal white matter damage and subsequent cerebal palsy, and the occurrence of premature low birth weight deliveries.

4.9.1. Prophylactic Methods

In one aspect, the invention provides a method for preventing in a subject, a disease or condition associated with an aberrant IL-1L1 expression or activity by administering to the subject an agent which modulates IL-1L1expression or at least one IL-1L1 activity. Subjects at risk for such a disease can be identified by a diagnostic or prognostic assay, e.g., as described herein. Administration of a prophylactic agent can occur prior to the manifestation of symptoms characteristic of the IL-1L1aberrancy, such that a disease or disorder is prevented or, alternatively, delayed in its progression. Depending on the type of IL-1L1aberrancy, for example, a IL-1L1 agonist or IL-1L1antagonist agent can be used for treating the subject prophylactically. The prophylactic methods are similar to therapeutic methods of the present invention and are further discussed in the following subsections.

4.9.2. Therapeutic Methods

In general, the invention provides methods for treating a disease or condition which is caused by or contributed to by an aberrant IL-1L1 activity comprising administering to the subject an effective amount of a compound which is capable of modulating an IL-1L1 activity. Among the approaches which may be used to ameliorate disease symptoms involving an aberrant IL-1L1 activity are, for example, antisense, ribozyme, and triple helix molecules described above. Examples of suitable compounds include the antagonists, agonists or homologues described in detail herein.

4.9.3. Effective Dose

Toxicity and therapeutic efficacy of such compounds can be determined by standard pharmaceutical procedures in cell cultures or experimental animals, e.g., for determining The Ld50 (The Dose Lethal To 50% Of The Population) And The Ed50 (the dose therapeutically effective in 50% of the population). The dose ratio between toxic and therapeutic effects is the therapeutic index and it can be expressed as the ratio LD50/ED50. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

4.9.4. Formulation and Use

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres which offer the possiblity of local noninvasive delivery of drugs over an extended period of time. This technology utilizes microspheres of precapillary size which can be injected via a coronary chatheter into any selected part of the e.g. heart or other organs without causing inflammation or ischemia. The administered therapeutic is slowly released from these microspheres and taken up by surrounding tissue cells (e.g. endothelial cells).

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

In clinical settings, a gene delivery system for the therapeutic IL-1L1 gene can be introduced into a patient by any of a number of methods, each of which is familiar in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g., Chen et al. (1994) PNAS 91: 3054-3057). An IL-1L1gene, such as any one of the sequences represented in the group consisting of SEQ ID Nos. 1, or 4 or the IL-1L1 alternative 5′ ends represented by SEQ ID Nos. 2 or 3 or a sequence homologous thereto can be delivered in a gene therapy construct by electroporation using techniques described, for example, by Dev et al. ((1994) Cancer Treat Rev 20:105-115).

The pharmaceutical preparation of the gene therapy construct or compound of the invention can consist essentially of the gene delivery system in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical preparation can comprise one or more cells which produce the gene delivery system.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

4.10. Kits

The invention further provides kits for use in diagnostics or prognostic methods or for treating a disease or condition associated with an aberrant IL-1L1 protein. The invention also provides kits for determining which IL-1L1therapeutic should be administered to a subject. The invention encompasses kits for detecting the presence of IL-1L1 mRNA or protein in a biological sample or for determining the presence of mutations or the identity of polymorphic regions in an IL-1L1 gene. For example, the kit can comprise a labeled compound or agent capable of detecting IL-1L1 protein or mRNA in a biological sample; means for determining the amount of IL-1L1 in the sample; and means for comparing the amount of IL-1L1 in the sample with a standard. The compound or agent can be packaged in a suitable container. The kit can further comprise instructions for using the kit to detect IL-1L1 mRNA or protein.

In one embodiment, the kit comprises a pharmaceutical composition containing an effective amount of an IL-1L1 antagonist therapeutic and instruction for use in treating or preventing hypertension. In another embodiment, the kit comprises a pharmaceutical composition comprising an effective amount of an IL-1L1 agonist therapeutic and instructions for use in treating insect bites. Generally, the kit comprises a pharmaceutical composition comprising an effective amount of an IL-1L1agonist or antagonist therapeutic and instructions for use as an analgesic. For example, the kit can comprise a pharmaceutical composition comprising an effective amount of an IL-1L1agonist therapeutic and instructions for use as an analgesic.

Yet other kits can be used to determine whether a subject has or is likely to develop a disease or condition associated with an aberrant IL-1L1 activity. Such a kit can comprise, e.g., one or more nucleic acid probes capable of hybridizing specifically to at least a portion of an IL-1L1 gene or allelic variant thereof, or mutated form thereof.

4.11. Additional Uses for IL-1L1 Proteins and Nucleic Acids

The IL-1L1 nucleic acids of the invention can further be used in the following assays. In one embodiment, the human IL-1L1 nucleic acid having SEQ ID No. 1 or a portion thereof, or a nucleic acid which hybridizes thereto can be used to determine the precise chromosomal localization of an IL-1L1gene within the IL-1 locus. Furthermore, the IL-1L1 gene can also be used as a chromosomal marker in genetic linkage studies involving genes other than IL-1L1.

Chromosomal localization of a gene can be performed by several methods well known in the art. For example, Southern blot hybridization or PCR mapping of somatic cell hybrids can be used for determining on which chromosome or chromosome fragment a specific gene is located. Other mapping strategies that can similarly be used to localize a gene to a chromosome or chromosomal region include in situ hybridization, prescreening with labeled flow-sorted chromosomes and preselection by hybridization to construct chromosome specific-cDNA libraries.

Furthermore, fluorescence in situ hybridization (FISH) of a nucleic acid, e.g., an IL-1L1 nucleic acid, to a metaphase chromosomal spread is a one step method that provides a precise chromosomal location of the nucleic acid. This technique can be used with nucleic acids as short as 500 or 600 bases; however, clones larger than 2,000 bp have a higher likelihood of binding to a unique chromosomal location with sufficient signal intensity for simple detection. Such techniques are described, e.g, in Verma et al., Human Chromosomes: a Manual of Basic Techniques, Pergamon Press, New York (1988). Using such techniques, a gene can be localized to a chromosomal region containing from about 50 to about 500 genes.

If the IL-1L1gene is shown to be localized in a chromosomal region which cosegregates, i.e., which is associated, with a specific disease, the differences in the cDNA or genomic sequence between affected and unaffected individuals are determined. The presence of a mutation in some or all of the affected individuals but not in any normal individuals, will be indicative that the mutation is likely to be causing or contributing to the disease.

The present invention is further illustrated by the following examples which should not be construed as limiting in any way. The contents of all cited references (including literature references, issued patents, published patent applications as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization(B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### 5. EXAMPLES

5.1. Cloning and Analysis of Human and Murine IL-1L1

In order to find new genes within the interleukin-1 gene cluster (Nicklin et al., 1994) with possible functions in human inflammatory responses, we obtained yeast bearing the 680 kb CEPH yeast artificial chromosome clone 766E12, which contains the IL-1 cluster (Nothwang et al., 1996) and isolated the chromosome by pulsed field gel electrophoresis. We used it as a driver in the selection method for cDNA described by Morgan et al. (1992). cDNA was derived from human peripheral blood mononuclear cells which were cultured for 6 hr in the presence of 10 ng/ml phorbol myristate acetate and 100 ng/ml E coli lipopolysaccharide. cDNA was also amplified as described and cDNA fragments were cloned into pNEB193 after two rounds of affinity selection and amplification. A random sample of one hundred were sequenced entirely. Sequences were assembled into overlapping units. The IL-1L1expressed sequence tag (EST) corresponded to one of these overlapping sequences, derived from three overlapping fragments (e031 and e049 in my notes) making a contiguous sequence of 530 bp. It also overlapped two reported EST clones, R70089 and R00741. On testing, it was found to hybridise specifically with P1 artificial chromosome 131J6 from the Gingrich human PAC library, which maps between the IL1B and IL1RN genes adjacent to ICRF 700G1305 from the ICRF human P1 library (Nothwang et al., 1996). This latter clone contains the IL-1ra gene (IL1RN). No open reading frame was present either in the database EST sequences nor in our own novel EST sequences.

EST DNA was used as a probe to isolate a cDNA from the H9 placenta cDNA plasmid library from HGMP. The clone was only 1.6 kb, but was polyadenylylated, but again contained no coding sequence. We sequenced the entire clone and used the 5′ end sequence to perform a primer dependent amplification of the 5′ end of the IL-1L1cDNA from placenta. The product was approximately 1.1 kb and overlapped the sequence of the cDNA clone. We have since used a more 5′ set of primers for the amplification reactions and have obtained >5 separate isolates which seem to contain two different non-coding 5′ ends (at least two isolates of each), which are shown in FIG. 1. The total predicted length of the mRNA is now 2.6 kb. The coding sequence occupies less than the first 600 nucleotides. With the exception of a likely cloning error, all cDNA sequences (of which we have entirely sequenced three) have encoded the same protein, which appears to have no leader sequence. Certain, human cDNAs have been reported that are identical to the sequences that we report here (Mulero, et al. (1999) Biochem Biophys Res Commun. 263: 702-706; Smith, et al. (2000) J. Biol. Chem. 275: 1169-1175; Busfield, et al. (2000) Genomics 66: 213-216)

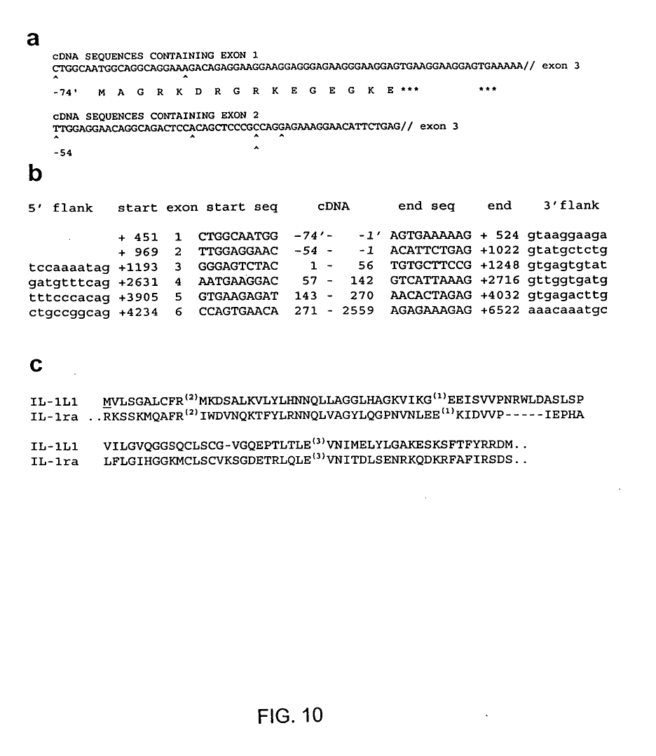

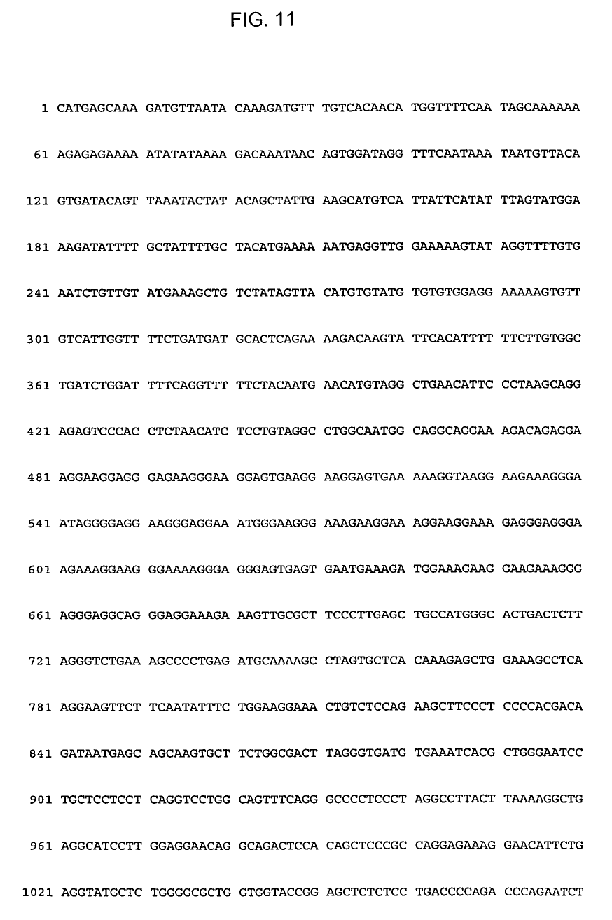

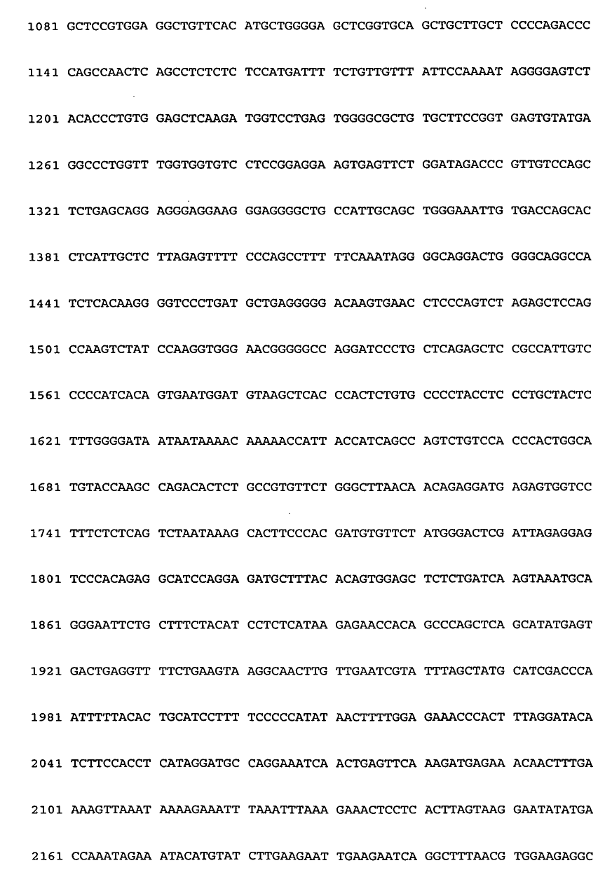

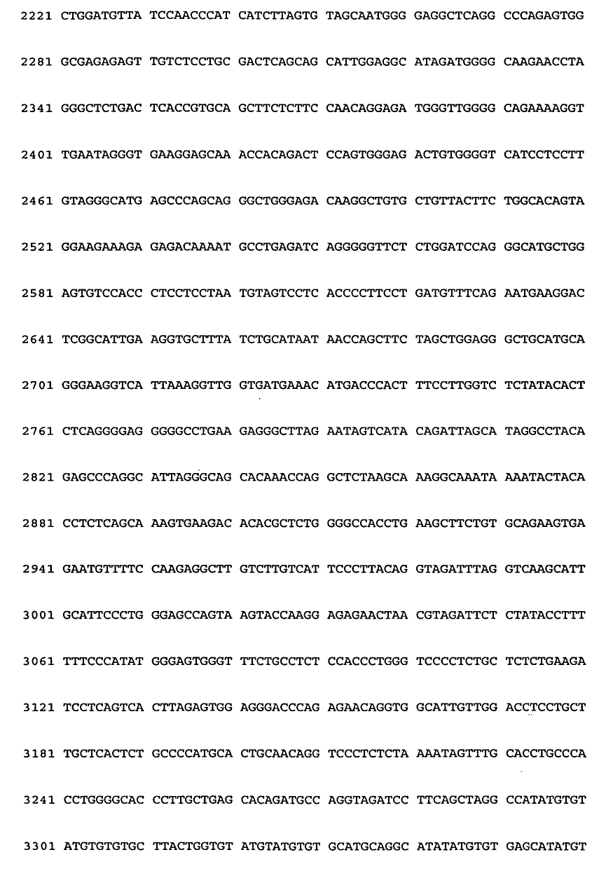

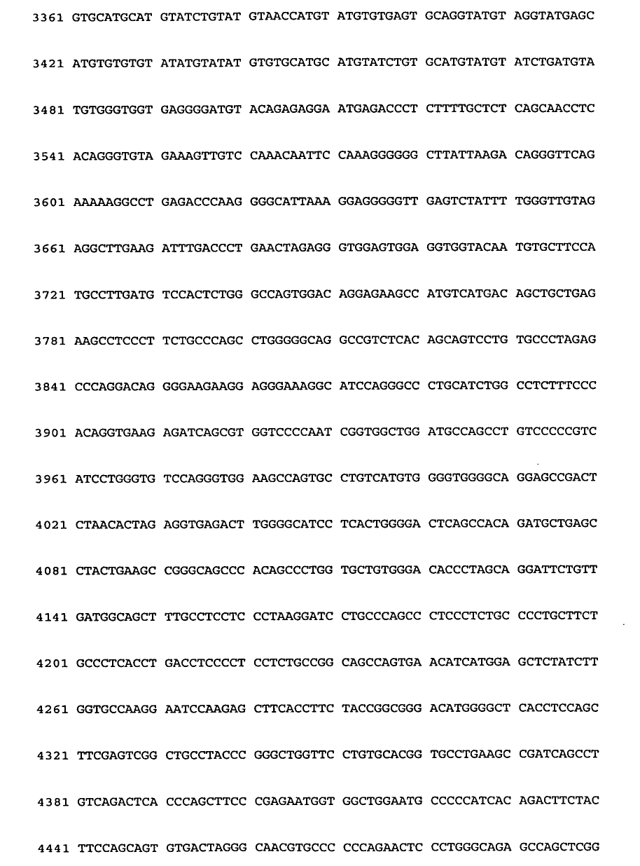

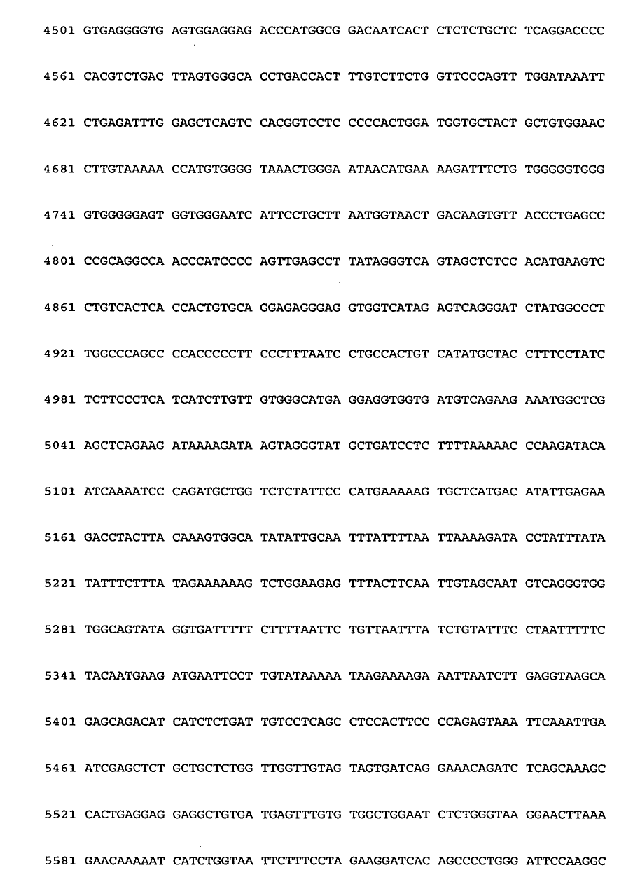

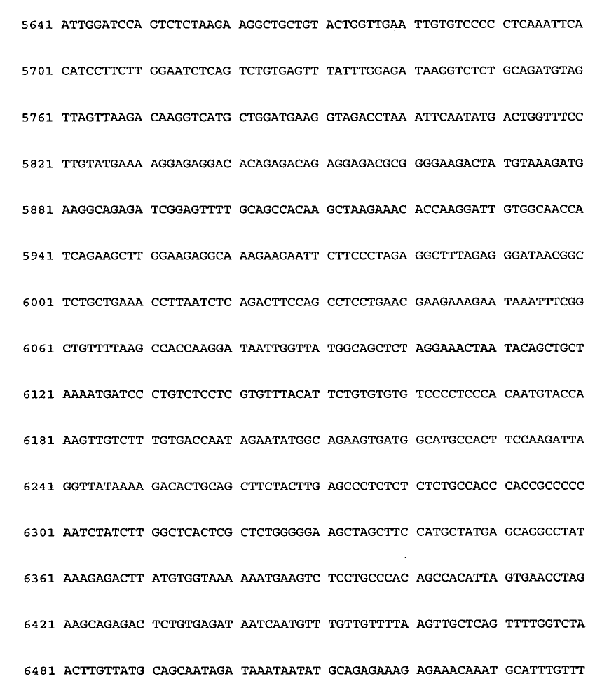

The genomic Sequence and Mapping of IL1L1. Subclones of PAC 131J6 were generated from Xba I and EcoR I-digested DNA, and were sequenced by primer walking. Good sequence was obtained at least once from both strands. The IL1L1 gene spans 6072 nucleotides and is illustrated in FIG. 11. The positions of the intron-exon boundaries are shown in FIG. 10(b) and correspond closely with those of IL1RN, as has been previously described for other members of the IL-1 structural family. The total sequenced segment from PAC 131J6 (6540 nucleotides, accession AJ271338) contains six exons, from 450 nucleotides upstream of exon 1 to 18 nucleotides downstream of the polyadenylylation site. A High Throughput Genomic Sequence of PAC RPII-339F22 (accession AC016724) has recently also been deposited on the EMBL database. Our genomic sequence deviates from it at 44 positions (0.67%) after rechecking our primary data for discrepancies. PAC RP11-339F22 contains a 195 kb genomic insert that at present has been sequenced in 12 non-overlapping fragments. We placed 11 of 12 fragments in order using the positions of previously established markers (Hildebrandt, et al. (1996) Cytogenet. Cell Genet. 73: 235-239) and the deposited sequence of the IL1RN locus. By restriction mapping and hybridization with 5′ and 3′ probes from IL1L1, we were able to place the remaining fragment and orientate the others. IL1L1 is therefore placed between STS markers 10H8S and 131P6S, and is transcribed towards 131P6S. Assuming that any omitted sequence from AC016724 is small (as suggested by the three gaps in IL1RN) then we conclude that the polyadenylylation signal of IL1L1 lies ˜53 kb 5′ of the first exon of IL1RN and that the two genes are in the same orientation. From our previous mapping studies, this would imply a separation of ˜270 kb between IL1L1 and IL1B.

Because a break has occurred in the IL-1 cluster between human and mouse (Zahedi, et al. (1991) J. Immunol. 146: 4228-4233), we considered whether the difference in IL-1L1 transcripts between the species might result from a disruption within exon 6 of mouse Il1l1. We screened mouse PAC clones from the 129/Sv RPCI21 library (Ioannou, et al. (1996) In Dracopoli et al (Eds.) Current Protocols in Human Genetics. Wiley New York, 5.15.1-5.15.24) with a mouse IL-1L1 cDNA probe, and obtained three plasmids, two of which also contained Il1rn ORFs, providing clear evidence that Il1l1 and hIm are neighbors, and that the syntenic break has occurred between Il1b and Il1l1 rather than Il1l1 and Il1rn. Low stringency hybridization of a “Zoo” blot (Clontech Inc.) provided evidence that the IL-1L1 gene is present in all eutherian species (FIG. 8).

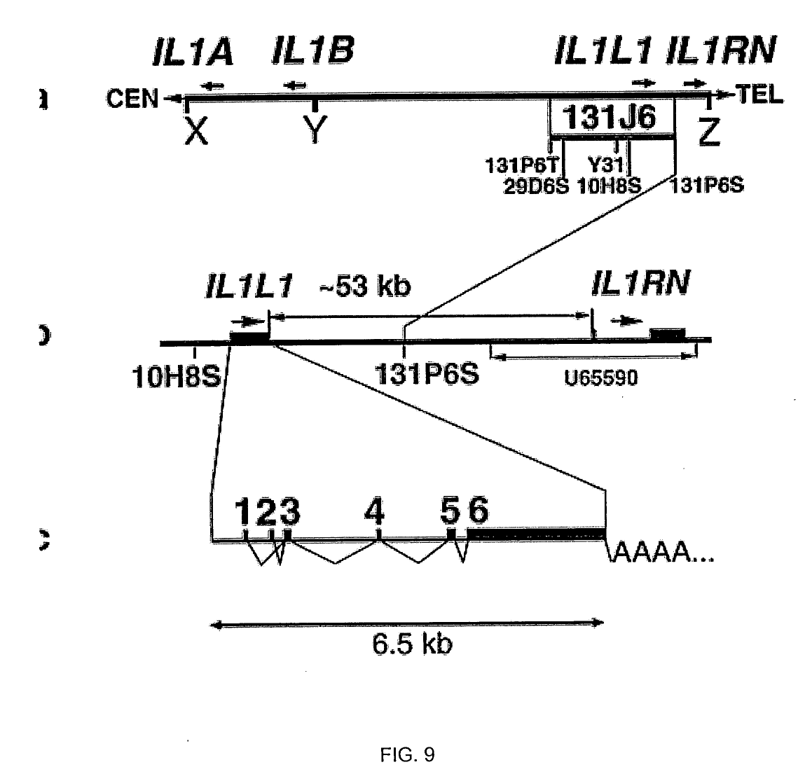

FIG. 9 shows the mapping of the IL1L1 locus within the IL-1 Gene Cluster. FIG. 9(a) illustrates the approximate position of the IL1L1 in relation to the three mapped genes IL1A, IL1B and IL1RN. The overall length of the cluster was from restriction mapping in relation to the CpG islands X, Y and Z. The positions of the markers and of PAC 131J6 are indicated. FIG. 9(b) shows more detailed mapping of the first exon of IL1L1 in relation to the intracellular-form first exon of IL1RN (IL1Rnex1′) and the flanking markers 10H8S and 131P6S. The extent of known sequence from the IL1RN locus is indicated (accession U65590). (c) Exon distribution within the IL1L1 gene derived from sequencing the gene. Bold numbers indicate the positions of the respective exons.

Alternative 5′ splicing of IL-1L1 mRNA was assessed. Six out of seven 5′ RACE products that we examined were clearly cloned independently because of differences in their first residues (FIG. 10(a)). The set of seven fell into groups of five and two which had entirely different stretches at their 5′ ends and appear to originate from alternative first exons (exons 1 and 2, see FIG. 9 and FIG. 10(c)). We presume that the two exons have alternative promoters, both lacking TATAA boxes. Exon 1 contains an out-of-frame upstream ORF of 16 codons (shown in FIG. 10(a)) followed by a stop codon; an arrangement which might be relevant to the regulation of translation of IL-1L1.

FIG. 10 shows certain details of the splicing of IL-1L1Transcripts. (a) Use of two apparent first exons (exons 1 and 2) in mRNA synthesis. Seven independent cDNA cloning events from 5′ RACE of placental mRNA were sequenced. The longest cDNA sequence found is shown for both the rarer (exon 1 initiated) and commoner (exon 2 initiated) mRNA forms. Arrows indicate start points of cDNA sequences. (Note that real transcripts must commence with an A or G). A short upstream out-of-frame ORF with AUG in good context is implied in exon 1 (and is shown translated as single letter code). (b) Intron-exon boundaries of the IL1L1 gene. “+1” in the cDNA sequence is defined as the beginning of the coding exon 3. In the absence of a fixed transcriptional start, genomic sequence positions are arbitrarily defined in relation to the genomic sequence presented in database submission AJ271338. “5′ flank” and “3′ flank” indicate the respective flanking sequences. “Start” and “end” indicate the first and last bases of the exon. “Exon” indicates the exon number. “Start seq.” and “end seq.” indicate the sequences at the start and end of the exon. “cDNA” gives the cDNA sequence encoded within the exon. (c) Illustration of the conservation of splice sites between human IL-1L1and its closest homolog, IL-1ra, in relation to the aligned translation products of the two genes. Positions of splice sites are indicated by parenthetized superscripted numbers representing the nucleotide within the triplet of the previous codon that acts as the splice donor.

FIG. 11 shows the complete genomic sequence of the human IL1L1 gene. Full-length IL-1L1cDNA sequences have EMBL accession numbers AJ242737 and AJ242738. The full sequence IMAGE cDNA clone #332733, encoding mouse IL-1L1is accession AJ250429. The human IL1L1 genomic clone shown in FIG. 11 is accession AJ271338.

5.2. Tissue Distribution of IL-1L1mRNA

The expression of IL-1L1gene was assessed by Northern blotting. Commercial blots of poly(A)+ tissue mRNA were surveyed with a probe corresponding to the IL-1L1ORF. Only placenta gave a strong signal, consisting of 2.7 kb and much fainter 1.2 kb bands (FIG. 7(a)). A small amount of mRNA appeared to be present in thymus. The initial EST which we used to identify IL-1L1 was derived from endotoxin and PHA-stimulated PBMC, so we sought to define the possible source. Leukocyte classes were fractionated and total RNA was hybridized with the IL-1L1ORF. IL-1L1mRNA of the expected size was detected clearly in endotoxin-stimulated monocytes and in vitro-differentiated macrophages. It appeared to be absent from NK cells, lymphocytes and from in vitro-differentiated dendritic cells (FIG. 7(b)). Much stronger expression of IL-1ra is seen in all of these leukocyte fractions. IL-1L1 mRNA is relatively strongly expressed in differentiated macrophages. Using RT-PCR, we have also detected expression of the ORF of IL-1L1 in human skin, brain, heart, kidney, leukocytes, placenta and spleen but not in liver.

A Northern blot was hybridized with the IL-1L1 probe and revealed a corresponding mRNA of an approximately 2.7 kb transcript in placenta. FIG. 7 shows the detection of IL-1L1 mRNA in human tissues. (a) Northern blot hybridization of the IL-1L1 ORF with 5 μg/lane polyA+ RNA (Clontech Inc.) isolated from the following tissues. Pa, pancreas; Ki, kidney; Mu, skeletal muscle; Li, liver; Lu, lung; Pl, placenta; Br, brain; He, heart; Le, peripheral blood leukocytes; Co, colon; In, intestine; Ov, ovary; Te, testis; Pr, prostate; Th, thymus; Sp, spleen. An arrow on the left of the main panel indicates the positions of the 2.6 kb IL-1L1mRNA. The positions of RNA molecular size markers are indicated on the right. The top panel is an exposure of the same blot after hybridization with a control b-actin probe. (b) The same IL-1L1 probe as above was hybridized to a Northern blot containing total mRNA isolated from the following cells: Ly+, lymphocytes stimulated for 48 hr with 5 μg/ml PHA; Mc−, unstimulated monocytes; Mc+, monocytes activated with 100 ng/ml LPS; Pm−, unstimulated PMN; Pm+, PMN stimulated with LPS (100 ng/ml) for 4 hr; MF, macrophages cultivated for 6 days in autologous serum; NK−, unstimulated NK cells; NK+, NK cells activated with IL-2 for 48 hr; DC-, immature dendritic cells; DC+, mature dendritic cells; HE−, unstimulated HUVEC; HEI, HUVEC treated with 6 nM IL-1b for 4 hr; HEL, HUVEC treated with 100 ng/ml LPS for 4 hr. The image shown is of a ten day exposure of the blot. The blot was subsequently probed with the ORF of IL-1ra, a control gene that responds to inflammation, and b-actin as a loading control. The positions of the ribosomal RNAs are indicated on the main panel.

5.3. Analysis of IL-1L1 Polypeptide Sequence and Structure

The protein product of the IL-1L1gene is predicted to share 47% identical residues with the human interleukin-1 receptor antagonist protein (see FIG. 5). A further sequence homology search revealed a partial cDNA sequence from a mouse clone in the IMAGE database (332733) which encoded a partial polypeptide sequence which appeared to be the mouse isologue of the human IL-1L1protein sequence. We obtained the clone and sequenced its insert entirely. A final comparison of the putative mouse and human IL-1L1 proteins, and the human IL-1L1 and IL-1ra proteins is shown (see FIG. 4). The IMAGE cDNA is also polyadenylylated, but is grossly divergent from the human IL-1L1cDNA sequence in its 3′ non-coding region and the mRNA is presumably only 1.2 kb, which is far shorter than the human mRNA. So far, we have been unable to detect IL-1L1 mRNA on a Northern blot of mouse tissues. In the human gene, the divergent sequence occurs within the final very long exon 4. Hybridisation with a human IL-1 L1-specific probe on a Southern blot of a number of genomic DNA digests (mainly mammals) at reduced stringency revealed a single clear homologous band in all mammals tested, but none in yeast and no clear signal in chicken (see FIG. 8). The intron-exon structure resembles that of the other members of the IL-1 family.

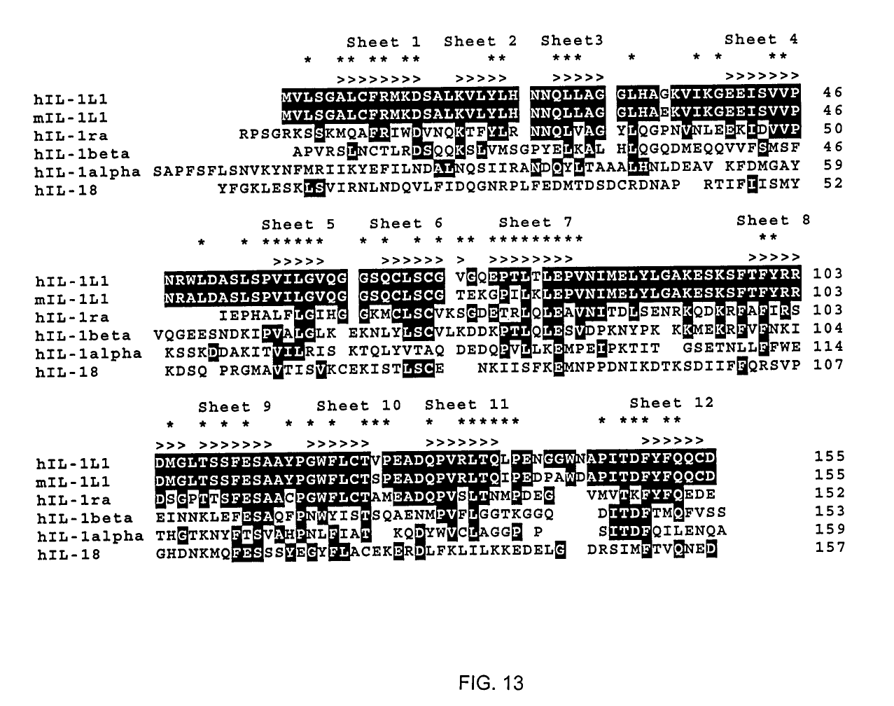

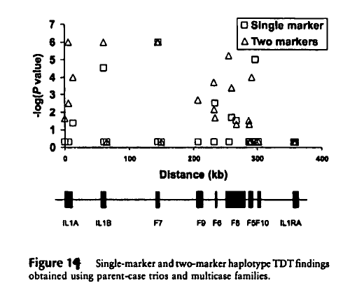

Comparisons of the novel open reading frame of human IL1L1 reveals that human IL-1L1 shares 73/155 residues (47%) with the processed secreted form of human IL-1ra, and 42/155 (27%) residues with human IL-1b (FIG. 13). BLAST (Altschul, et al. (1990) J. Mol. Biol. 215: 403-410) searching also revealed a fragmentary open reading frame, accession W08205, corresponding to IMAGE clone #332733, which clearly contained an ORF of the mouse isolog of IL-1L1. We sequenced the clone completely. The mouse ORF starts with two AUG codons. By context and analogy with the human sequence, it is likely that the second AUG would be preferred for initiation. Mouse and human IL-1L1 ORFs would be 90% identical (FIG. 13). However, although the clone #332733 contains both the ORF and is polyadenylylated, it is only 1.2 kb in length, and its sequences diverge entirely from the 2.7 kb human cDNA 34 nt after translational termination. Though we have detected expression of the mouse IL-1L1ORF by reverse transcription and PCR of mRNA from RAW264.7 mouse macrophages, we have not yet confirmed its length by Northern blot hybridization.

We have prepared human IL-1 receptor antagonist protein and IL-1L1 precursors with the following predicted sequences ( see FIG. 6) and have isolated them by nickel chelate chromatography followed by elution with imidazole and processing with thrombin to yield the following putative polypeptides. Preliminary nuclear magnetic resonance studies on ¹⁵N+¹³C double-labeled IL-1L1 protein indicated that the protein is folded largely as b-sheet, and analysis has indicated that the structure is consistent with a close physical resemblance to human IL-1ra.

The folding of recombinant IL-1 protein was further assessed by detailed NMR structural studies. In part, we have investigated the folding of rhIL-1L1, in order to ensure that the protein was apparently biologically inactive because it was misfolded. Single dimension ¹H NMR spectra of Forms A and B indicated well folded monomeric proteins. We found that Form A was insufficiently soluble for multidimensional NMR methods unless the artificial N-terminal extension was retained. However, the 1-D spectrum of uncleaved Form A precursor IL-1L1 overlapped well with cleaved Form B (which was used in the full set of biological experiments) indicating an identical conformation. We prepared ¹³C, ¹⁵N double-labeled, uncleaved Form A precursor (LH, MN and John Waltho, unpublished data). The shifts of alpha ¹³C-carbons were determined. Blocks of alpha carbons with up-field shifts >0.7 ppm indicate the presence of b-sheets (Wishart, et al. (1994) NMR, 4: 171-180) and are shown by asterisks in FIG. 13. These results correlate well with the positions of b-sheet residues in IL-1ra, which are indicated by chevrons in FIG. 13. We conclude that processed Form B rhIL-1L1and uncleaved Form A are folded in a closely similar way and that the folding pattern is essentially the same as that of its nearest protein homolog, IL-1ra.

FIG. 13 shows the alignment of human and mouse IL-1L1sequences with those of the other known members of the IL-1 family. Alignments were selected to retain homology blocks and conserve structural features present in IL-1b and IL-1ra. The core regions of the conserved beta sheets of IL-1ra are indicated above the alignment with chevrons. Asterisks indicate residues of IL-1L1 whose ¹³C-labelled alpha carbons have up-field shifts >0.7 ppm. Concentrations of such residues indicate b-sheet structure. Shaded residues are conserved, in this alignment, between human IL-1L1 and the target sequence.

5.4. IL-1L1 Antibodies and Immunoprecipation Assays

The recombinant IL-1L1 protein described above was also used to raise antibodies in rabbits.

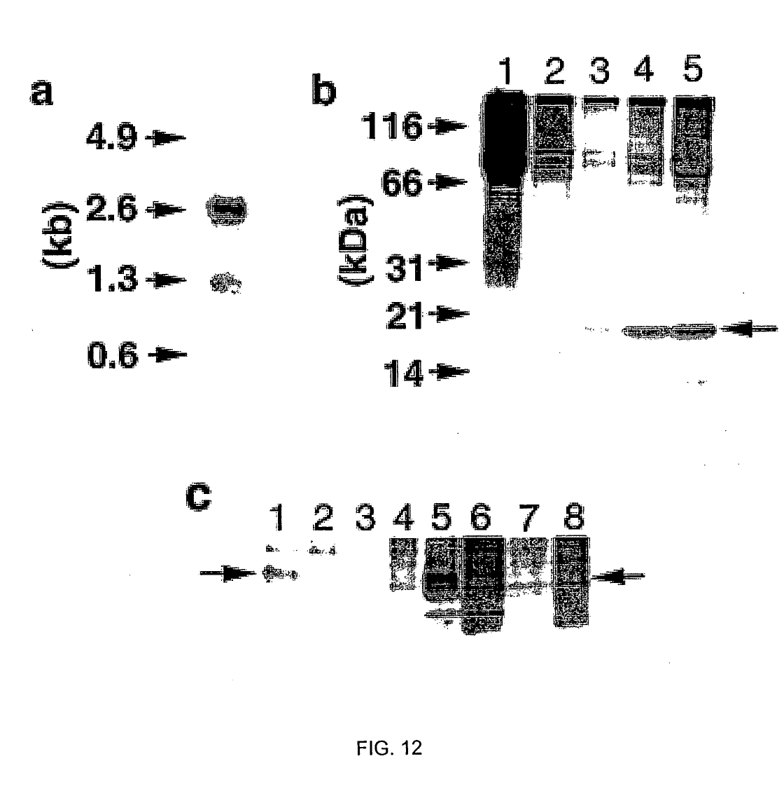

We showed that the choriocarcinoma cell line JEG-3 produces IL-1L1 mRNA constitutively. We labeled the protein metabolically and showed that a 17 kDa protein could be immunoprecipitated specifically with the rabbit anti-IL-1L1polyclonal antiserum. Expression of the IL-1L1protein was detected by immunoprecipitation. The trophoblastic cell line JEG-3 (Kohler, et al. (1971) J Clin Endocrinol. 32: 683-687) appeared to contain the IL-1L1 2.7 kb mRNA constitutively (FIG. 12(a)), as might be expected if the major cellular component of the placenta, the trophoblasts, express IL-1L1. Specific polyclonal serum to recombinant human (rh)IL-1L1 was raised in rabbits. JEG-3 were metabolically labeled and cell lysates were prepared. Immunoprecipitation revealed a specific radioactive band with a mobility corresponding to a protein of 17 kDa (FIG. 12(b) lanes 3-5); a control whole serum did not immunoprecipitate the same product. (FIG. 12(b) lane 1). Pre-competition of the antibody with rhIL-1L1 prevents immunoprecipitation of the 17 kDa band, further suggesting that this represents the endogenous IL-1L1protein (compare FIG. 12(c) lanes 1 with 2 and 5 with 6). Unlike the IL-1ra protein, IL-1L1 lacks N-glycosylation signals, and the electrophoretic mobility of the endogenous protein in comparison to the material prepared in E. coli appears identical, confirming a lack of modification.

FIG. 12 depicts the immunprecipitation of native IL-1L1 protein from JEG-3 cells. (a) Detection of constitutive IL-1L1 mRNA in JEG-3 cells. A Northern blot of PolyA+ RNA selected from 2×107 JEG-3 cells was probed with an IL-1L1 ORF cDNA probe. The arrow indicates the 2.7 kb mRNA. The position of RNA size-markers are shown to the left. (b) Extracts of metabolically radiolabelled cells were immunoprecipitated with, lane 1, 20 μl of an irrelevant antiserum; lane 2, no serum; lane 3, 5 μl; lane 4, 10 μl, and lane 5, 20 μl of anti-IL-1L1 antiserum. The mobility of protein molecular mass standards are indicated on the left. The arrow on the right of the picture indicates the likely band of IL-1L1 protein. Immunoprecipitation of this band was not observed after blocking the antibody with rhIL-1L1. (c) Evidence for secretion of IL-1L1from JEG-3 cells. Cell lysates (lanes 1-4) and supernatants (lanes 5-8) were immunoprecipitated with, lanes 1 and 5, 40 μl anti-IL-1L1 antiserum; lanes 2 and 6, with 20 μl anti IL-1L1antiserum pre-blocked with 20 μg rhIL-1L1; lanes 3 and 7, but with an irrelevant serum; and lanes 4 and 8, with no serum. Part of the gel is shown. The arrows mark the position of migration of rhIL-1L1. The supernatant contained only 15% of the amount of cell-associated total TCA-precipitable radioactivity.

5.5. IL-1L1 Cell Based Assays for IL-1 Activity

The possible influence of IL-1L1on IL-1-like signaling was investigated with a simple assay to determine its effect on production of IL-6, a cytokine that sensitively and directly responds to IL-1 (Zilberstein, et al. (1986) EMBO J. 5: 2529-2537). Three forms of IL-1L1 were generated as recombinant 6His-tagged fusion proteins in E. coli which were digested with thrombin (Smith, et al. (1988) Gene 67: 31-40) to release the required protein. Form A omits the initiator Met- codon and replaces it with Gly-Ser-Ser- (see FIG. 6). In case the precise nature of the N-terminal was important, we also generated a Form B that contained Met1 and Form C that began with Val2 (see FIG. 6). For comparison, IL-1ra was prepared and isolated identically and was also modified by removal of the first two post-cleavage residues, Pro-Lys- and addition of Gly-Ser-Ser. All three forms of IL-1L1 were tested in preliminary experiments for their IL-1-like activity in stimulating primary fibroblasts to secrete IL-6, which was detected by an ELISA. We performed a detailed study of the effect of Form B IL-1L1 on fibroblasts and endothelial cells. At up to 0.1 μM IL-1L1 had <1% of the activity of 0.1 nM IL-1b. These data place an upper limit of <1/105 on the IL-1-like activity of IL-1L1.

The activity of IL-1L1 was also compared with IL-1ra in the same systems. Initial dose response experiments (data not shown) demonstrated that 10 nM IL-1ra protein caused >95% reduction in IL-6 production from fibroblasts in response to 0.1 nM IL-1b. Again, preliminary experiments were done with all three forms. We then used IL-1ra or IL-1L1 at 0.1 μM in competition with 10 nM IL-1a or IL-1b (R&D Systems Inc.). The detailed study was again made with Form B of IL-1L1in comparison with IL-1ra. IL-1ra completely eliminated the production of detectable IL-6, but IL-1L1had no strong reproducible effect on the activity of either form of IL-1. A minor and non-significant reduction in the IL-6 output from fibroblasts was not increased in pilot experiments done with 1 μM IL-1L1. Before concluding that IL-1L1 was not a receptor antagonist, we used supernatants from COS-7 cells transfected with the IL-1L1 expression vector described above, as a possible source of a more naturally processed protein. In duplicate experiments, no effect of ˜20 nM COS-generated IL-1L1 (determined from Western blots of the cell conditioned medium) upon signaling by 0.1 nM IL-1b could be detected on fibroblasts, neither could any agonistic effect be observed. Conditioned medium from cells transfected with empty vector was used in control tests. IL-1ra at 20 nM was found to eliminate the effect of IL-1b. We therefore concluded that IL-1L1 had no direct effect on the IL-1 system.

The activity of rhIL-1L1 in the IL-18 system was tested in the human myelomonocytic cell line KG-1, which has been reported to produce IFNg in response to IL-18 stimulation (Konishi, et al. (1997) J Immunol Methods 209: 187-19)). We used a modified assay in which cells are stimulated with PHA for two hours prior to IL-18 stimulation. We tested Form B rhIL-1L1 as both an IL-18-like agonist and as an antagonist. The presence of both PHA and IL-18 resulted in a 3-5 fold increase in the production of IFNg from the cells, compared to PHA alone. rhIL-1L1 was clearly unable to substitute for IL-18 in this assay. Furthermore rhIL-1L1was unable to block the effect of IL-18 in this system when compared with a monoclonal antibody against IL-18. We do note a small reduction in the IFNg output from cells stimulated with PHA alone on addition of 0.1 μM IL-1L1. Although IL-18 assays are relatively insensitive and variable compared with those for IL-1, our results do suggest that IL-1L1 also has no direct role in the IL-18 system.

The occurrence of IL-1L1receptors on fibroblasts was next assessed. IL-1 receptors are present on fibroblasts at very low abundance. To test whether the absence of a biological response to IL-1L1was likely to result from the absence of a receptor, we needed to develop an extremely sensitive binding assay for IL-1L1and IL-1ra, as a control. Both IL-1L1and IL-1ra were prepared with the N-terminal extension (after thrombin cleavage) GSSGLRRA*SLGSS (where the asterisk indicates a substrate serine residue for protein kinase A). The two extended proteins were named KA-IL-1ra and KA-IL-1L1. KA-IL-1ra lacked the first two residues of the processed secreted protein (as did the functional rhIL-1ra used in our biological experiments). KA-IL-1L1lacked only the initiator methionine. The proteins were phosphorylated with protein kinase A (New England BioLabs) to high specific activity (1-2×10¹⁷ Bqmol-1). In a series of triplicate experiments on fibroblasts, we were able to demonstrate binding of KA-IL-1ra that could be competed by 0.1 μM IL-1ra. Typically, specific binding was ˜100 Bq/4×10⁴ cells, or ˜2000 receptors per cell (in line with previous estimates). By comparison, there were no detectable (<200/cell) receptors for KA-IL-1L1on fibroblasts (data not shown).

5.6. Expression of Recombinant IL-1L1in COS Cells

We produced a recombinant human IL-1L1 in mammalian cells, using the original translational start of the IL-1L1mRNA. The 1.4 kb IL-1L1 5′ RACE cDNA was fused downstream of a SV40 promoter and synthetic intron and was followed by an SV40 polyadenylation signal (pSG5, Stratagene Inc.). COS7 cells transfected with this construct yielded a protein of the expected size. Western blot analysis (data not shown) of cell lysates and supernatants with the same anti-IL-1L1 antiserum showed a specific 17 kDa protein (which was not present in untransfected samples) that was ˜3-fold more abundant in the supernatant than in the cytoplasmic fraction. This suggested that IL-1L1, like other members of the IL-1 family, might have an unconventional secretion pathway. We confirmed this finding by detecting the endogenous IL-1L1 in the cell-bound and supernatant fractions of JEG-3 cell in culture. Cells were metabolically labelled, as before and supernatants and cytoplasmic preparations were compared by immunoprecipitation. From FIG. 12(c), it can be seen that despite the presence of less than 15% of total TCA-precipitable radioactivity in the JEG-3 supernatant, compared with a cell lysate, there is substantially more IL-1L1in the supernatant, demonstrating that IL-1L1 is selectively released from cells, which suggests a signal-independent secretion pathway.

In general, an expression construct containing a nucleic acid encoding a full length human IL-1L1 protein, or a soluble IL-1L1 protein which is devoid of the signal sequence can be constructed as follows. A nucleic acid encoding the full length human IL-1L1 protein or a soluble form of IL-1L1 protein described above is obtained by reverse transcription (RT-PCR) of mRNA extracted from human cells The PCR primers further contain appropriate restriction sites for introduction into the expression plasmid. The amplified nucleic acid is then inserted in a eukaryotic expression plasmid such as pcDNAI/Amp (InVitrogen) containing: 1) SV40 origin of replication, 2) ampicillin resistance gens, 3) E. coli replication origin, 4) CMV promoter followed by a polylinker region, a SV40 intron and polyadenylation site. A DNA fragment encoding the full length human IL-1L1 and a HA or myc tag fused in frame to its 3′ end is then cloned into the polylinker region of the. The HA tag corresponds to an epitope derived from the influenza hemagglutinin protein as previously described (I. Wilson, H. Niman, R. Heighten, A Cherenson, M. Connolly, and R. Lemer, 1984, Cell 37, 767). The infusion of HA tag to IL-1L1allows easy detection of the recombinant protein with an antibody that recognizes the HA epitope.

For expression of the recombinant IL-1L1, COS cells are transfected with the expression vector by DEAE-DEXTRAN method. (J. Sambrook, E. Fritsch, T. Maniatis, Molecular Cloning: A Laboratory Manual, Cold Spring Laboratory Press, (1989)). The expression of the IL-1L1 -HA protein can be detected by radiolabelling and immunoprecipitation with an anti-HA antibody. (E. Harlow, D. Lane, Antibodies: A Laboratory Manual, Cold Spring Harbor Laboratory Press, (1988)). For this, transfected cells are labeled with ³⁵S-cysteine two days post transfection. The cells, or alternatively the culture media (e.g., for the soluble IL-1L1) is then collected and the IL-1L1 protein immunoprecipitated with an HA specific monoclonal antibody. To determine whether full length IL-1L1 is a membrane protein, and/or a secreted protein, the cells transfected with a vector encoding the full length IL-1L1 protein can be lysed with detergent (RIPA buffer (150 mM NaCl 1% NP-40, 0.1% SDS, 1% NP-40, 0.5% DOC, 50 mM Tris, pH 7.5). (Wilson, I. et al., Id. 37:767 (1984)). Proteins precipitated can then be analyzed on SDS-PAGE gel. Thus, the presence of IL-1L1in the cell will be indicative that the full length IL-1L1 can be membrane bound and the presence of IL-1L1in the supernatant will be indicative that the protein can also be in a soluble form, whether produced as a secreted protein or released by leakage from the cell.

5.7. Materials and Methods

Identification of a novel transcript. PBMC were stimulated for either 6 hr or 18 hr with 100 ng/ml E coli lipopolysaccharide and 100 μg/ml PHA. RNA was prepared after both treatments and mixed. cDNA was selected by two rounds of solution phase hybridization essentially as described (Morgan, et al. (1992) Nucleic Acids Res. 20: 5173-5179; Futreal, et al. (1994) Hum. Mol. Genet. 3: 1359-1364) with the 680 kb YAC 766E12 (obtained from CEPH, Paris) (Chumakov, et al. (1995) Nature 377 (Suppl):175) as a driver. 100 clones were sequenced individually. Sequences containing high copy-number elements were rejected.

cDNA and Genomic DNA Libraries. Human genomic PAC clone 131J6 has been described (Nothwang, et al. (1997) Genomics 41: 370-378). Human placental cDNA library H9, a primary ligation mixture in pCDM8, was obtained from the Resource Centre HGMP (Cambridge, UK). Mouse PAC clones were obtained from the Resource Centre HGMP (Cambridge, UK) after screening a gridded mouse 129 PAC library (RPCI21) with a 1.2 kb mouse IL-1L1 cDNA probe from IMAGE clone #332733 (Lennon, et al. (1996) Genomics 33: 151-152).

Isolation of a cDNA clone. Approximately 5×105 colonies from the human H9 cDNA library were screened by standard procedures with a 474 bp probe from an EST clone, which maps to nucleotides 5902 to 6369 of the genomic sequence (FIG. 2) in exon 6. Two clones were identified, screened to homogeneity and found to contain similar sized inserts.

5′-Extension of the cDNA. First-strand cDNA was prepared from placental mRNA with MMLV-reverse transcriptase and an oligo(dT) primer. Second-strand synthesis was done with a cocktail of E.coli DNA polymerase I, RNase H, and E. coli DNA ligase and cDNA adapters were ligated (according to the manufacturers instructions. Marathon RACE; Clontech Inc.). 5′ RACE was performed with an adapter specific primer and a gene specific primer complementary to 5295-5270 of the genomic sequence (30 cycles at 680 C), followed by nested PCR with the adapter nested primer and a primer corresponding to nt 5273-5244 (20 cycles at 600 C). The 1.4 kb 5′ RACE product was cloned directly into pCRII vector (Invitrogen) and sequenced with specific oligonucleotide primers.

Northern blot Analysis. Human multiple tissue blots were purchased from Clontech Laboratories, Inc. and contained S pig of mRNA per lane. To isolate mRNA from JEG-3 cells, 2×107 cells were homogenized into RNAzol B (Biogenesis) and extracted according to the manufacturer's instructions. JEG-3 poly(A)+ RNA was isolated from total RNA by affinity chromatography on oligo(dT)-cellulose type 3 (Promega). Leukocytes were fractionated by standard density-centrifugation protocols (Colotta, et al. (1984) J. Immunol. 132: 936-944; Bertani, et al. (1989) Blood 74: 1811-1816). NK cells were obtained from PBMCs by depletion of CD3+ and CD14+ cells. Dendritic cells were obtained by treatment of monocytes with GM-CSF and IL-4 for 7 days (Sozzani, et al. (1997) J. Immunol. 159: 1993-2000). Functional maturation was achieved by stimulation with TNF, IL-1b or LPS for 3-4 hours. Total RNA from leukocytes was prepared by the guanidine thiocyanate method. RNA was resolved by electrophoresis in 1.2% formaldehyde/agarose gels, and transferred to charged nylon membranes (Zeta-Probe, Biorad Inc.). IL-1L1 mRNA was visualized by hybridization with a cDNA probe corresponding to the IL-1L1 ORF.

Preparation of recombinant proteins. Forms A, B and C of rhIL-1L1, rhIL-1ra, KA-IL-1L1and KA-IL-1ra were expressed as 6His-tagged recombinant proteins in BL21(DE3) cells from a modified pET plasmid which added a 6His tag to the proteins' N-termini, a flexible linker and a thrombin cleavage site. Bacteria were cultured as described (Studier, et al. (1990) Methods Enzymol. 185: 60-89), chilled before being induced with 0.5 mM isopropyl-b-D-thiogalactopyranoside (IPTG) and shaken 18 hr at 25 [symbol] C. Harvested bacteria were resuspended in 100 mM NaCl/20 mM Tris/HCl pH 7.3/10% glycerol (Buffer A) and sonicated in the presence of 1 mM phenylmethanesulfonylfluoride. Lysates were cleared at 600000× g for 20 min and the His6-tagged protein from a 1000 ml culture was affinity purified on a 5 ml Ni-NTA agarose column (Qiagen). The column was washed successively with 50 ml Buffer A, 50 ml Buffer A/20 mM imidazole and the tagged protein was eluted in the minimum volume of Buffer A/100 mM imidazole. The proteins were digested with thrombin (3 units/mg fusion protein) for 4 hours at 30 [symbol] C, and then separated from the N-terminal fragment by gel filtration on Sephadex G-75 in Buffer A/2 mM dithiothreitol. Initially, possible contamination by LPS was removed from protein samples by affinity with polymyxin B-agarose column (Sigma). Later, because no significant effect that could be due to LPS was detected, protein was used without this last step. Purity was determined by gel electrophoresis and concentration was assessed from A280 measurement adjusted for the amino-acid composition of the proteins. The purified proteins appeared homogenous with the exception of a trace of undigested material in the preparation of rhIL-1L1 Form C, presumably because of steric hindrance from the b-branched Val residue, and signs of some further C-terminal processing products in KA-IL1L1. Generation of anti-IL-1L1 sera. Dwarf lop-eared rabbits were injected five times at four-week intervals with 150 μg of rhIL-1L1Form A in Freund's adjuvant. Serum samples were tested by enzyme-linked immunosorbent assay (ELISA) with IL-1L1.

Metabolic labeling of JEG-3 cells and immunoprecipitation. Cells (80% confluent) in a 75 cm2 tissue culture bottle were washed with isotonic saline and placed in cysteine/methionine free MEM (Life Technologies)/1 mM glutamine/1 mM sodium pyruvate/0.5% bovine serum albumin, for 1 hour at 37 [symbol] C. Following this, fresh medium, 5 ml, supplemented with 50 mCi/ml of (³⁵S)cysteine/methionine (Promix, NEN Life Sciences) was added and the cells incubated for 4 hours. A further 50 mCi/ml (³⁵S)cysteine/methionine was added and incubation continued for 20 hours. The medium was collected. In FIG. 12(b), cells were washed and lysed in 1.0 ml of RIPA buffer (100 mM NaCU/50 mM Tris/HCl pH 8.0/1% Nonidet P-40/0.5% sodium deoxycholate/0.1% sodium dodecylsulfate) containing a protease inhibitor cocktail (Boehringer Mannheim), and centrifuged at 13000 g at 4 [symbol] C. to remove particulate debris. Lysate was pre-cleared for 1 hour at 4 [symbol] C. in 0.5 ml samples with 20 μl (packed volume) Protein A-Sepharose beads (Sigma) and incubated with test serum at 4 [symbol] C. with gentle agitation for 24 hours, then 20 μl Protein A-Sepharose was added for a further 4 hour incubation. The solid matrix was collected and washed four times with RIPA buffer before resuspending it in 60 μl of sample buffer and heating to 100 [symbol] C. for 2 min. A 30 μl sample was resolved by gel electrophoresis. In FIG. 12(c) the method was essentially the same but we used 2.5 ml of cell supernatant or cells lysed with 2.5 ml RIPA buffer.

Investigation of possible IL-1, IL-1ra and IL-18-like activities of IL-1L1in human cells. Human gingival fibroblasts were plated in 24-well tissue culture plates (2×104 per well) and grown for 24 hours in DMEM/10% fetal bovine serum. Human umbilical vein endothelial cells (TCS Biologicals) were plated on 24 well tissue culture plates and grown until 80% confluent as described by the suppliers. Cells were stimulated for 24 hours with fresh medium that contained where appropriate, IL-1a, IL-1b (R&D Systems); rhIL-1ra or rhIL-1L1 (prepared as described). Previous experiments (not shown) indicated that 0.1 nM of either IL-1 was adequate for a robust activation of IL-6 secretion, while 10 nM IL-1ra was sufficient to reduce the response to 0.1 nM IL-1 by >95%. IL-6 production was measured by ELISA as described in the manufacturers instructions (R&D Systems) within the linear range of the assay. Day-to day variation in IL-6 output from similar experiments, possibly because of variation in cell density or small changes in the activity of the IL-1 species, was corrected by normalizing each set of data against the mean output from the panel of IL-1-treated wells that were produced in the same experiment. Observed variation, over a number of experiments, in IL-6 output from single well cultures of fibroblasts treated with IL-1 alone was 7-30 ng/ml for both IL-1a and IL-1b, and in endothelial cells, 1.3-7.0 ng/ml.

KG-1 cells (ATCC CCL-246) were plated on 24 well tissue culture plates at 1×106/ml in Iscove's modified Dulbecco's medium (Life Technologies), 20% fetal bovine serum with or without 50 mg/ml PHA (Sigma) for 2 hours. Cells were stimulated for 48 hours with PHA alone or together with IL-18 (3 nM) and/or IL-1L1(0.1 μM). To demonstrate that enhanced IFNg production was dependent upon IL-18, neutralizing anti-IL-18 antibody (50 μg/ml, R&D Systems) was added to some tests. Supernatants were centrifuged at the end of the stimulation period at 900× g for 5 min to remove cells. IFNg production was determined by ELISA (Biosource). Sets of data were standardized against the mean output of four wells, in the same experiment, that had been stimulated with both PHA and IL-18. The range of IFNg output in PHA/IL-18 stimulated cells was 2.3-10.8 i.u./ml in two experiments of four wells.

Association of IL-1L1 with Susceptibility to Ankylosing Spondylitis

Ankylosing spondylitis (AS [MIM 106300]) affects 1-9 in 1,000 British white individuals, making it one of the most common causes of inflammatory arthritis after rheumatoid arthritis (van der Linden et al. 1983; Braun et al. 1998). The condition affects predominantly the axial skeleton, including the spine and sacroiliac joints, and causes pain, stiffness, and, eventually, bony ankylosis. Joints and tendon insertions (entheses) elsewhere are also commonly involved, and approximately one-third of patients develop acute anterior uveitis. Familial clustering has implicated genetic factors in the disease etiology; heritability, as assessed by twin studies, is >90% (Brown et al. 1997), and the sibling recurrence risk ratio is 53-82 (Brown et al. 2000b). The major susceptibility gene in AS is HLA-B27 (Brewerton et al. 1973). HLA-B27 carriage is present in >95% of white individuals with AS, yet only 1%-5% of HLA-B27 carriers develop AS, and HLA-B27 carriage alone does not explain the pattern of disease recurrence in families (Brown et al. 2000b). Since familial AS is in nearly all cases restricted to those carrying HLA-B27, it has been postulated that HLA-B27 is almost essential but not adequate to cause AS and that other genes interact with HLA-B27 to cause the condition (Brown et al. 2002). Although several areas of suggestive or significant linkage have been reported from whole-genome scans, only one non-MHC gene, CYP2D6 (MIM 124030) on chromosome 22, has had replicated association reported with AS (Beyeler et al. 1996; Brown et al. 2000a).

The chromosome 2q13 region has been linked to AS in a whole-genome screen (Laval et al. 2001), with a maximum LOD score of 2.5 at a position 132 cM from the p-telomere. The magnitude of the sibling recurrence risk due to this locus is A=1.7 (95% CI 1.3-2.3), equivalent to 12% of the familiality of AS. The interleukin 1 (IL-1) family gene cluster lies 123-126 cM from the p-telomere. Association of alleles of the IL-1RN (MIM 147679) variable number of tandem repeats (VNTR) with AS has been reported in some studies (McGarry et al. 2001; van der Paardt et al. 2002) but not others (Djouadi et al. 2001). Maksymowych et al. (2003) demonstrated association between AS and SNPs and SNP haplotypes within IL-1RN. The prototypic members of the IL-1 family gene cluster are the genes IL-1A (MIM 147760), IL-1B (MIM 147720), and IL-1 RN. IL-1 A and -B encode proinflammatory cytokines involved in host defense against infection. The IL-1 receptor antagonist IL-1RA, encoded by the gene IL-1 RN, is an anti-inflammatory nonsignalling molecule that competes for receptor binding with IL-1 a and IL-1β. Six genes with structural homology to IL-1A/B or IL-1RN lie between IL-1A and IL-1RN. They are named IL-1FS (MIM 605507), IL-1F6 (MIM 605509), IL-1F7 (MIM 605510), IL-1F8 (MIM 605508), IL-1F9 (MIM 605542), and IL-1F10; IL-1A, IL-1B, and IL-1 RN have been renamed IL-1 F1, IL-1F2, and IL-U3, respectively (Sims et al. 2001). The genes are ordered, from centromere to telomere, as IL-1A, IL-1B, IL-1F7, IL-1F9, IL-1F6, IL-1F8, IL-1F5, IL-U10, IL-1 RN, in a cluster within an -360-kb region (Nicklin et al. 2002).

Given these findings and the prominent role of proteins encoded by genes lying within this complex in inflammation and in host defense against infection, the association of IL-1 complex genes with AS was investigated. (See Timms et al., Am. J. Human Genetics 75:587-95 (2004) (“Timms”), incorporated herein by reference in its entirety. In Timms, SNPs were identified in all nine IL-1 cluster gene members and, along with a VNTR in IL-1 RN, were genotyped in families and unrelated controls. Timms demonstrated strong transmission disequilibrium of alleles and haplotypes of these markers in two separate family collections and demonstrate association by comparing cases from these families and unrelated healthy controls. These findings indicate that the IL-1 gene cluster contains a major susceptibility locus for AS.

Subjects, Material, and Methods

Families and Control Individuals

The Timms study included 227 white British families containing affected sibling pairs and 317 parent-case trios with AS, as well as 200 healthy blood donors. AS was defined by the modified New York diagnostic criteria (van der Linden et al. 1984). The diagnosis of AS was confirmed, in all cases, by a qualified rheumatologist and was followed by either examination or telephone interview. In cases with an atypical history or no previous radiographic evidence, pelvic and lumbosacral spine radiographs were obtained and attending general practitioners were contacted to confirm diagnosis. All cases were HLA-1127 positive. There were 930 cases (66% male), who were, on average, 43.9 years of age and had been affected for 21.6 years. The study was approved by the Multicentre Research Ethics Committee, and all participants gave informed written consent.

SNP Identification and Selection

Timms identified SNPs in the IL-1 gene cluster by using a combination of literature searches and public SNP databases. IL-1A, IL-1B, and IL-1RN have previously been extensively studied for polymorphisms and were therefore not sequenced further. For the remaining six genes, SNPs in exons, in exon-intron boundaries, or in the 5′ UTR of each gene were identified from SNP databases and then were verified by direct sequencing in 48 healthy controls. An EST lying between IL-1F7 and IL-IF9 (locus 129359) was also studied. Because of strong linkage disequilibrium (D′>0.9) with other IL-1 RN markers, the SNPs IL-1RN8061 and IL-1 RN9S89 were not analyzed further. Twenty-five SNPs with minor-allele frequencies ≧5% were used for the subsequent studies, in addition to the IL-1 RN VNTR.

Genotyping Methods

SNPs were genotyped by Timms either by Sequenom MassArray (Sequenom) or PCR/restriction digestion. The SNPs genotyped and the respective genotyping methods are given in Timms table 1, below. Alleles of the IL-1 RN 46-bp VNTR were characterized by PCR and separation by electrophoresis on 2% agarose gels.

Statistical Analysis

Timms indicated that all markers were genotyped in duplicate, and only consistent genotypes were accepted. In the multicase and single-case families, Mendelian inheritance was checked using the program GAS version 2 (A. Young, unpublished software). MERLIN (Abecasis et al. 2002) was used to identify unlikely recombination events suggestive of genotyping error; such genotypes were repeated and rejected if the experimental data were still equivocal. Genotype and allele frequencies were compared in the probands of the multicase and single-case families. Where these differed by >10%, the marker concerned was regenotyped.

Analysis consisted of examination of within-family association of single markers and two-marker haplotypes of contiguous polymorphisms in parent-case and affected-sibling-pair families, using TRANSMIT (Clayton and Jones 1999). P values were determined by bootstrap simulation, as implemented within TRANS-MIT, to account for the linkage component when using multicase families; significance levels therefore reflect association rather than linkage (Clayton and Jones 1999). For each analysis, 10⁶ simulations were performed, so that the minimum P value reported is P<10⁻⁶, which we have recorded as P=0. The TRANSMIT bootstrap simulation method is described at the TRANSMIT Web site.

A case-control analysis was also performed by Timms, comparing allele and genotype frequencies in probands from the affected sibling-pair families and parent-case trio families with findings in unrelated healthy control individuals. In this analysis, all markers were tested individually; specific two-marker haplotypes were also assessed, as outlined in the “Results” section. Haplotypes in unrelated control samples were determined using the program PHASE (Stephens and Donnelly 2003), a Bayesian haplotype re-construction method; haplotypes were included in further analysis if they were assigned >90% certainty. For case-control haplotype comparisons, haplotypes were determined in the parent-case trio and affected-sibling-pair families through use of the program GENE-HUNTER and proband haplotypes (i.e., one per family) used for the cases (Kruglyak et at. 1996). Pairwise linkage disequilibrium was calculated using the program GOLD, through use of haplotype data from the healthy controls (Abecasis and Cookson 2000).

Timms also employed stepwise conditional logistic regression (Cordell and Clayton 2002) and the conditional extended transmission/disequilibrium test (CETDT) (Koeleman et al. 2000) to determine whether one or more SNPs could be identified as the primary associated variant(s). According to Timms, this analysis was performed using all members of the parent-case trio and affected-sibling-pair families. These methods use a conditional logistic regression approach to investigate the interdependence of associated physically linked markers. Markers are entered or deleted from the conditional logistic regression equation in a stepwise manner, to determine whether a marker is important once the effects of other markers have already been accounted for. Phase-known genotype and haplotype relative risks (HRRs) can also be estimated without bias when this approach is used (Cordell and Clayton 2002). The CETDT can be considered to be a special case of the conditional logistic regression in which the effect of alleles is assumed to be multiplicative. The CETDT has the advantage of allowing for missing parental genotypes via an EM algorithm, but this utility can be prohibitively slow to implement when more than a few markers are being considered simultaneously. Also, the CETDT does not correct for any nonindependence between multiple affected offspring from the same family, which is accounted for within the stepwise conditional logistic regression method by use of robust Huber-White information sandwich variance estimation, allowing all affected individuals to be studied within multicase families.

Timms et al.: IL-1 Genes in Ankylosing Spondylitis

TABLE 1
Markers Studied and the Genotyping Methods Employed

| | | GENOTYPING | PRIMER^{a} | | ENZYME/EXTENSION |
|---|---|---|---|---|---|
| MARKER | SNP ID | METHOD | Forward | Reverse | PRIMER |
| IL-1A559 | 3783559 | Digest | tggtctctcatttgtctcatc | tgttgcttgctcagtgttttg | MseI |
| IL-1A376 | 2071376 | Digest | ccaagatgaagaccaaccag | aggtcaggcagggaaaggaa | BstYI |
| IL-1A889 | 1800587 | Digest | ctttaataatagtaaccaggcaaga | gcccaaggtgtgtcttctgt | DpnII |
| IL-1B3953 | 1143634 | Digest | gtaaataggctgaaaggggaaa | gtaaataggctgaaaggggaaa | TaqI |
| IL-1B5810 | 1143633 | Digest | gtatatgctcaggtgtcctc | catggagaattagcaagctg | Fnu4HI |
| IL-1B-511 | 16944 | Digest | acttaagttaggaatcttcccact | gagcttatctccagggttgc | AvaI |
| IL-1F7-1 | 2723187 | Sequenom | acgttggatggcattagcctcatccttgag | acgttggatgaaactcccctttagagaccc | ctctgcggagaaaggaagtc |
| IL-1F7-2 | 2708947 | Sequenom | acgttggatgcttttattagggctcaggtg | acgttggatgaattgcaggaggtgcagatg | gggctcaggtgggctcc |
| LOCUS 129359 | 2708954 | Digest | agggctcactcgaatgactt | atggggtaggaggagtgacc | BspHI |
| IL-1F9-1 | 2121334 | Sequenom | acgttggatgagccagattgcaaagactag | acgttggatgggtgaagtgaacattccctg | tgcatggacatctacaaatggc |
| IL-1F6-1 | 1446512 | Sequenom | acgttggatgtctagagtccattgaggtgg | acgttggatgatgctgcttctccatggaag | gagctggctttgctacagtaa |
| IL-1F6-2 | 1446511 | Sequenom | acgttggatgagcttctcagagccactgtg | acgttggatgtcaggagctggctttgctac | gcatgctgcttctccatgg |
| IL-1F6-3 | 895497 | Sequenom | acgttggatcacacccgatgattgatatc | acgttggatgtgactttctcaacagcattg | tgatatcctgaatgctcccc |
| IL-1F8-1 | 1562304 | Sequenom | acgttggatggtggaaaagaggcttgttag | acgttggatgctctcctgttacacactcag | gaggcttgttagagaggagg |
| IL-1F8-2 | 2197578 | Digest | ggactgacagtggagcaaca | ggcctaacatcagatgcaaa | BsmI |
| IL-1F8-3 | 1900287 | Sequenom | acgttggatgctacactgtgcaacttcagc | acgttggatggggaaaatcatctgcatggg | cttcagcctgcccacctta |
| IL-1F5-1 | 990524 | Sequenom | acgttggatgaagaaggaagaaagggaggg | acgttggatgtcagaccctaagagtcagtg | gaggcagggaggaaagaaagt |
| IL-1F5-2 | 2441375 | Digest | ggaaattgtgaccagcacct | gagctctagactgggaggttcaatt | MfeI |
| IL-1F5-3 | 1530548 | Digest | gtaagctcacccactctgtgcccgt | acatgccagtgggtggac | RsaI |
| IL-1F5-4 | 1374286 | Sequenom | acgttggatggtgggcaggtgcaaactatt | acgttggatgattgttggacctcctgcttg | tgcaaactattttagagagggac |
| IL-1F10-1 | 996878 | Sequenom | acgttggatggtctctttataggcctgctc | acgttggatgcccaatctatcttggctcac | aggcctgctcatagcatgg |
| IL-1F10-2 | 1446522 | Sequenom | acgttggatggaatctctggatcttgctcc | acgttggatgccttcatcattctcctgcac | tctggatcttgctccacagaa |
| IL-1F10-3 | 3811058 | Digest | caaggtggtgattcagagca | gggaggaaagggaagaagag | BsmAI |
| IL-1RN8006 | 419598 | Digest | ttctatctgaggaacaaccaactagtagc | caccagacttgacacaggacaggca | HpaII |
| IL-1RN8061^{b} | 423904 | Digest | acacaggaaggtgccaagca | tgcagacagacgggcaaagt | MwoI |
| IL-1RNVNTR | | Digest | ctcagcaacactcctat | tcctggtctgcaggtaa | 46-bp repeat |
| IL-1RN9589^{b} | 454078 | Digest | ttgtggggaccaggggagat | agcctggcactctgctgaat | SspI |
| IL-1RN11100 | 315952 | Digest | agggaggcagcacaggactt | agtccctgcagtccttgcca | MspAI |

^{a}Underlined bases are deliberate mismatches.
^{b}Studied in AS family probands only; not assessed further because of strong linkage disequilibrium with other IL-1RN markers (D′ > 0.9).

TABLE 2
Within-Family TDT Results when Individual Markers and Two-
Marker Haplotypes Are Used

| Marker | One-Marker P Value | Two-Marker P Value^{a} |
|---|---|---|
| IL-1A559 | NS | .022 |
| IL-1A376 | NS | .003 |
| IL-1A889 | NS | 0 |
| IL-1B3953 | .04 | .0001 |
| IL-1B5810 | NS | 0 |
| IL-1B-511 | .00003 | 0 |
| IL-1F7-1 | NS | NS |
| IL-1F7-2 | NS | .0003 |
| LOCUS 129359 | NS | NS |
| IL-1F9-1 | NS | NS |
| IL-1F6-1 | NS | .002 |
| IL-1F6-2 | NS | .0002 |
| IL-1F6-3 | NS | .007 |
| IL-1F8-1 | .003 | .02 |
| IL-1F8-2 | NS | .000006 |
| IL-1F8-3 | .02 | .0004 |
| IL-IF5-1 | .03 | .05 |
| IL-1F5-2 | NS | .03 |
| IL-1F5-3 | NS | NS |
| IL-IF5-4 | NS | .05 |
| IL-1F10-1 | NS | NS |
| IL-1F10-2 | NS | .007 |
| IL-1F10-3 | .003 | NS |
| IL-1RN8006 | NS | NS |
| IL-1RNVNTR | NS | NS |
| IL-1RN11100 | NS | NS |

NOTE
NS = not significant.
^{a}Two-marker haplotypes consist of contiguous markers; P values are given with the 5′ marker of the two markers concerned.

Within-family studies demonstrated significant association of both single-marker and two-marker haplotypes for several markers spread across the IL-1 gene cluster (see Timms table 2). Seven markers achieved significance levels of P<0.05 by within-family association. Two markers were sufficiently significant to remain so once Bonferonni correction was made for multiple (i.e., 26) comparisons (IL-1B-511, P=0.00003; IL-1F10-3, P=0.00001). Two-marker haplotypes gave the strongest associations, with 18 markers achieving P<0.05, 6 of which achieved P<10-6 (see Timms FIG. 1 and Timms table 2).

Case-control analysis by Timms confirmed either genotypic or allelic association for 10 markers (see table 3), all of which had been associated with AS in the within-family analysis, either as individual markers or as part of a two-marker haplotype.

TABLE 3
Genotype and Allele Frequencies in AS Cases
(Parent-Case Trios and Multicase Families) and Controls

| | | | | | | NO. (%) OF | |
|---|---|---|---|---|---|---|---|
| | | | | | | HEALTHY | |
| | NO. (%) OF CASES WITH | | | | | CONTROLS WITH | |
| | Genotype | | Allele | | | Genotype | |
| MARKER | 11 | 12 | 22 | 1 | 2 | 11 | 12 |
| IL-1A559 | 211 (53.7) | 142 (36.1) | 40 (10.2) | 564 (71.8) | 222 (28.2) | 112 (65.1) | 52 (30.2) |
| IL-1A376 | 29 (6.6) | 207 (46.9) | 205 (46.5) | 265 (30) | 617 (70) | 19 (11) | 77 (44.8) |
| IL-1A889 | 151 (41.6) | 174 (47.9) | 38 (10.5) | 476 (65.6) | 250 (34.4) | 87 (51.5) | 71 (42) |
| IL-1B3953 | 262 (54.6) | 193 (40.2) | 25 (5.2) | 717 (74.7) | 243 (25.3) | 98 (52.1) | 82 (43.6) |
| IL-1B5810 | 48 (9.8) | 241 (49.4) | 199 (40.8) | 337 (34.5) | 639 (65.5) | 9 (5) | 82 (45.8) |
| IL-1B-511 | 211 (41.3) | 256 (50.1) | 44 (8.6) | 678 (66.3) | 344 (33.7) | 76 (38.8) | 86 (43.9) |
| IL-1F7-1 | 449 (86.7) | 66 (12.7) | 3 (0.6) | 964 (93.1) | 72 (6.9) | 147 (88.6) | 18 (10.8) |
| IL-1F7-2 | 447 (84.5) | 77 (14.6) | 5 (0.9) | 971 (91.8) | 87 (8.2) | 161 (83) | 32 (16.5) |
| LOCUS 129359 | 415 (85.4) | 67 (13.8) | 4 (0.8) | 897 (92.3) | 75 (7.7) | 164 (85.9) | 26 (13.6) |
| IL-1F9-1 | 358 (68.8) | 151 (29) | 11 (2.1) | 867 (83.4) | 173 (16.6) | 132 (72.1) | 44 (24) |
| IL-1F6-1 | 220 (46) | 225 (47.1) | 33 (6.9) | 665 (69.6) | 291 (30.4) | 103 (56.3) | 66 (36.1) |
| IL-1F6-2 | 231 (45.2) | 253 (49.5) | 27 (5.3) | 715 (70) | 307 (30) | 94 (55) | 69 (40.4) |
| IL-1F6-3 | 262 (51.8) | 217 (42.9) | 27 (3.3) | 741 (73.2) | 271 (26.8) | 109 (68.1) | 41 (25.6) |
| IL-1F8-1 | 400 (84.2) | 74 (15.6) | 1 (0.2) | 874 (92) | 76 (8) | 172 (88.2) | 23 (11.8) |
| IL-1F8-2 | 214 (43.9) | 235 (48.3) | 38 (7.8) | 663 (68.1) | 311 (31.9) | 99 (53.5) | 69 (37.3) |
| IL-1F8-3 | 186 (43.6) | 209 (48.9) | 32 (7.5) | 581 (68) | 273 (32) | 102 (52.3) | 79 (40.5) |
| IL-1F5-1 | 184 (44.7) | 193 (46.8) | 35 (8.5) | 561 (68.1) | 263 (31.9) | 105 (53.6) | 79 (40.3) |
| IL-1F5-2 | 62 (14.8) | 216 (51.4) | 142 (33.8) | 340 (40.5) | 500 (59.5) | 52 (28.4) | 70 (38.3) |
| IL-1F5-3 | 82 (19.5) | 201 (47.7) | 138 (32.8) | 365 (43.3) | 477 (56.7) | 20 (11.3) | 82 (46.3) |
| IL-1F5-4 | 158 (37.7) | 211 (50.4) | 50 (11.9) | 527 (62.9) | 311 (37.1) | 69 (37.7) | 86 (47) |
| IL-1F10-1 | 413 (82.9) | 79 (15.9) | 6 (1.2) | 905 (90.9) | 91 (9.1) | 156 (83.4) | 28 (15) |
| IL-1F10-2 | 355 (77) | 101 (21.9) | 5 (1.1) | 811 (88) | 111 (12) | 147 (80.8) | 30 (16.5) |
| IL-1F10-3 | 404 (90.6) | 40 (9) | 2 (0.4) | 848 (95.1) | 44 (4.9) | 170 (90.4) | 18 (9.6) |
| IL-1RNVNTR | 256 (53.3) | 195 (40.6) | 29 (6) | 707 (73.6) | 253 (26.4) | 89 (53.3) | 70 (41.9) |

| | NO. (%) OF | | | | | |
|---|---|---|---|---|---|---|
| | HEALTHY CONTROLS WITH | | | | | |
| | Genotype | Allele | | P VALUE* | | ODDS |
| MARKER | 22 | 1 | 2 | Genotype | Allele | RATIO |
| IL-1A559 | 8 (4.7) | 276 (80.2) | 68 (19.8) | .016 | .003 | .63 |
| IL-1A376 | 76 (44.2) | 115 (33.4) | 229 (66.6) | | | |
| IL-1A889 | 11 (6.5) | 245 (72.5) | 93 (27.5) | | .025 | |
| IL-1B3953 | 8 (4.3) | 278 (73.9) | 98 (26.1) | | | |
| IL-1B5810 | 88 (49.2) | 100 (27.9) | 258 (72.1) | .049 | .023 | 1.36 |
| IL-1B-511 | 34 (17.3) | 238 (60.7) | 154 (39.3) | .0038 | .047 | 1.28 |
| IL-1F7-1 | 1 (0.6) | 312 (94) | 20 (6) | | | |
| IL-1F7-2 | 1 (0.5) | 354 (91.2) | 34 (8.8) | | | |
| LOCUS 129359 | 1 (0.5) | 354 (92.7) | 28 (7.3) | | | |
| IL-1F9-1 | 7 (3.8) | 308 (84.2) | 58 (15.8) | | | |
| IL-1F6-1 | 14 (7.7) | 272 (74.3) | 94 (25.7) | .037 | | .79 |
| IL-1F6-2 | 8 (4.7) | 257 (75.1) | 85 (24.9) | | | |
| IL-1F6-3 | 10 (6.3) | 259 (80.9) | 61 (19.1) | .0005 | .005 | .64 |
| IL-1F8-1 | 0 (0) | 367 (94.1) | 23 (5.9) | | | |
| IL-1F8-2 | 17 (9.2) | 267 (72.2) | 103 (27.8) | .039 | | .82 |
| IL-1F8-3 | 14 (7.2) | 283 (72.6) | 107 (27.4) | | | |
| IL-1F5-1 | 12 (6.1) | 289 (73.7) | 103 (26.3) | | .045 | |
| IL-1F5-2 | 61 (33.3) | 174 (47.5) | 192 (52.5) | .0002 | .023 | .75 |
| IL-1F5-3 | 75 (42.4) | 122 (34.5) | 232 (65.5) | .017 | .004 | 1.46 |
| IL-1F5-4 | 28 (15.3) | 224 (61.2) | 142 (38.8) | | | |
| IL-1F10-1 | 3 (1.6) | 340 (90.9) | 34 (9.1) | | | |
| IL-1F10-2 | 5 (2.7) | 324 (89) | 40 (11) | | | |
| IL-1F10-3 | 0 (0) | 358 (95.2) | 18 (4.8) | | | |
| IL-1RNVNTR | 8 (4.8) | 248 (74.3) | 86 (25.7) | | | |

*Significant P values (P < .05) are listed for alleles and geontypes.

To determine which marker or combination of markers was primarily responsible for the Timms also employed a stepwise conditional logistic regression approach (Cordell and Clayton 2002) (results were similar when the CETDT was used [Koeleman et al. 2000]). At the first stage, each marker was entered in turn into the regression equation, giving a 1-df test for the effect at a marker without accounting for effects at any other markers. This test is equivalent to performing a transmission/disequilibrium test (TDT) analysis at each marker in turn, using all families for which there are complete genotype data at the marker. Five markers were associated with AS in this analysis (IL-1113953, IL-1B-511, IL-1F6-2, IL-1F8-1, and IL-1F10-3; P=0.007, 0.0009, 0.047, 0.013, and 0.005 respectively). Each of these five markers, in turn, was accounted for by including the marker in the regression equation, and the effect of adding a second marker to the regression equation was examined for all the remaining markers, to test for their interdependence. Markers IL-1B-511 and IL-11′10-3 remained significant when added as second markers in the regression equation, regardless of which other of the five loci had been included as the first marker. If both of these markers were accounted for, under the assumption of multiplicative effects of haplotypes and applying Bonferroni correction for the number of markers studied, no other marker showed significant association with AS. When multiplicative effects of haplotypes are assumed in families with both parents genotyped, the combination of IL-11B-511 and IL-1F10-3 is significantly associated with AS (P=0.0002). HRRs, significance levels, and CIs are given in Timms table 4. Once IL-1B-511 was accounted for, IL-1F10-3 remained significantly associated (P=0.002), and vice versa (P=0.0008). Since this analysis used only families with both parents genotyped, the program TRANSMIT was used by Timms to test the association of this marker combination in all available families (both parent-case trio and affected-sibling-pair families). This demonstrated highly significant association (P=1.7×10⁻⁸). Further, when proband haplotypes established using the program GENEHUNTER and control haplotypes identified using the program PHASE were compared, significant association of this haplotypes was also observed (P=0.04).

TABLE 4
HRRs for combination of SNPs IL-1B-511 and IL-1F10-3
in Parent-Case Trios, Relative to Haplotype 2-2

| Haplotype | HRR | 95% CI | P Value |
|---|---|---|---|
| 1-1 | 11.3 | 2.6-49.8 | .001 |
| 1-2 | 8.7 | 1.9-39.1 | .005 |
| 2-1 | 5.8 | 1.3-27.5 | .028 |

According to Timms, linkage disequilibrium across the cluster was quite variable and appears to form three main blocks (see Timms table 5). Moderate linkage disequilibrium was observed between IL-1B-511 and IL-1F10-3 (D′=0.27; P=0.01).

Timms results indicate that a gene or combination of genes within the IL-1 gene cluster significantly influence susceptibility to AS. Several genetic studies have hinted at association of the IL-1 region with AS and, in particular, with polymorphisms of IL-1RN. Two groups have previously reported association between allele 2 of a VNTR in the IL-1 RN gene and AS (McGarry et al. 2001; van der Paardt et al. 2002). This polymorphism is associated with lower levels of IL-1RA production. Recently, Maksymowych and colleagues re-ported strong association of haplotypes of three SNPs within the 3′ region of the IL-1 RN gene with AS, but they did not study the IL-1 RN VNTR (Maksymowych et al. 2003). The fact that the haplotypic findings were stronger than individual SNP associations suggests either that these markers were not themselves involved in AS or that they acted in combination. The genes IL-1A, IL-1B, and IL-1 RN have been extensively sequenced in the past, and comprehensive data on their polymorphisms are available in public data-bases (University of Washington—Fred Hutchinson Cancer Research Center [UW FHCRC] Variation Discovery Resource Web site). Much less is known about genes IL-IFS-F10, including information about polymorphisms, expression patterns in tissues and disease, or function. The different IL-1 family members signal through a range of IL-1 receptors, with IL-1α/β signalling through IL-1R1 and IL-1R2, IL1-F6 signalling through IL-1R6, and IL-18 signalling through IL-1R5. IL-1F5 inhibits IL-1F6 activity, and IL-1F7 inhibits IL-18 activity. IL-1F8,-9, and -10 are thought to be IL-1 antagonists, since their sequences are most similar to IL-1RN, but their exact function is undetermined. IL-1F10 binds IL-1R1, suggesting that it may play a role in regulating IL-1A/B function (Smith et al. 2000). Although it appears that IL-1<<, IL-10, and IL-1RA are expressed at greater levels than other IL-1 gene family members, they are all widely expressed, including on activated monocytes and B cells (Laurincova 2000; Smith et al. 2000).

### References

Abecasis G R, Cherny S S, Cookson W O, Cardon L R (2002) Merlin-rapid analysis of dense genetic maps using sparse gene flow trees. Nat Genet 30:97-101
Abecasis G R, Cookson W O (2000) GOLD-graphical over-view of linkage disequilibrium. Bioinformatics 16:182-183
Beyeler C, Armstrong M, Bird H A, Idle J R, Daly A K (1996) Relationship between genotype for the cytochrome P450 CYP2D6 and susceptibility to ankylosing spondylitis and rheumatoid arthritis. Ann Rheum Dis 55:66-68
Brand J M, Neustock P, Kruse A, Alvarez-Ossorio L, Schnabel A, Kirchner H (1997) Stimulation of whole blood cultures in patients with ankylosing spondylitis by a mitogen derived from Mycoplasma arthritidis (MAS) and other mitogens. Rheumatol Int 16:207-211.
Braun J, Bollow M, Neure L, Seipelt E, Seyrekbasan F, Herbst H, Eggens U, Distler A, Sieper J (1995) Use of immuno-histologic and in situ hybridization techniques in the examination of sacroiliac joint biopsy specimens from patients with ankylosing spondylitis. Arthritis Rheum 38:499-505
Braun J, Bollow M, Remlinger G, Eggens U, Rudwaleit M, Distler A, Sieper J (1998) Prevalence of spondylarthropathies in HLA-B27 positive and negative blood donors. Arthritis Rheum 41:58-67
Brewerton D A, Hart F D, Nicholls A, Caffrey M, James D C, Sturrock R D (1973) Ankylosing spondylitis and HL-A 27. Lancet 1:904-907
Brown M A, Edwards S, Hoyle E, Campbell S, Laval S, Daly A K, Pile K D, Calin A, Ebringer A, Weeks D E, Wordsworth B P (2000a) Polymorphisms of the CYP2D6 gene increase susceptibility to ankylosing spondylitis. Hum Mol Genet 9: 1563-1566
Brown M A, Kennedy L G, MacGregor A J, Darke C, Duncan E, Shatford J L, Taylor A, Calin A, Wordsworth P (1997) Susceptibility to ankylosing spondylitis in twins: the role of genes, HLA, and the environment. Arthritis Rheum 40: 1823-1828
Brown M A, Laval S H, Brophy S, Calin A (20006) Recurrence risk modelling of the genetic susceptibility to ankylosing spondylitis. Ann Rheum Dis 59:883-886
Brown M A, Wordsworth B P, Reveille J D (2002) Genetics of ankylosing spondylitis. Clin Exp Rheumatol 20:S43-S49
Clayton D, Jones H (1999) Transmission/disequilibrium tests for extended marker haplotypes. Am J Hum Genet 65:1161-1169
Cordell H J, Clayton D G (2002) A unified stepwise regression procedure for evaluating the relative effects of polymorphisms within a gene using case/control or family data: application to HLA in type 1 diabetes. Am J Hum Genet 70: 124-141
Djouadi K, Nedelec B, Tamouza R, Genin E, Ramasawmy R, Charron D, Delpech M, Laoussadi S (2001) Interleukin 1 gene cluster polymorphisms in multiplex families with spondylarthropathies. Cytokine 13:98-103
Gratacos J, Collado A, Filella X, Sanmarti R, Canete J, Llena J, Molina R, Ballesta A, Munoz-Gomez J (1994) Serum cytokines (IL-6, TNF-alpha, IL-1 beta and IFN-gamma) in ankylosing spondylitis: a close correlation between serum I1-6 and disease activity and severity. Br J Rheumatol 33:927-931
Koeleman B P, Dudbridge F, Cordell H J, Todd J A (2000) Adaptation of the extended transmission/disequilibrium test to distinguish disease associations of multiple loci: the Conditional Extended Transmission/Disequilibrium Test. Ann Hum Genet 64:207-213
Kruglyak L, Daly M J, Reeve-Daly M P, Lander E S (1996) Para-metric and nonparametric linkage analysis: a unified multipoint approach. Am J Hum Genet 58:1347-1363
Laurincova B (2000) Interleukin-1 family: from genes to human disease. Acta Univ Palacki Olomuc Fac Med 143:19-29
Laval S H, Timms A, Edwards S, Bradbury L, Brophy S, Milicic A, Rubin L, Siminovitch K A, Weeks D E, Calin A, Words-worth B P, Brown M A (2001) Whole-genome screening in ankylosing spondylitis: evidence of non-MHC genetic-susceptibility loci. Am J Hum Genet 68:918-926
Maksymowych W P, Reeve J P, Reveille J D, Akey J M, Buenviaje H, O'Brien L, Peloso P M, Thomson G T, Jin L, Russell A S (2003) High-throughput single-nucleotide polymorphism analysis of the IL1RN locus in patients with ankylosing spondylitis by matrix-assisted laser desorption ionizationtime-of-flight mass spectrometry. Arthritis Rheum 48:2011-2018
McGarry F, Neilly J, Anderson N, Sturrock R, Field M (2001) A polymorphism within the interleukin 1 receptor antagonist (IL-1Ra) gene is associated with ankylosing spondylitis. Rheumatology (Oxford) 40:1359-1364
Nicklin M J, Barton J L, Nguyen M, FitzGerald M G, Duff G W, Kornman K (2002) A sequence-based map of the nine genes of the human interleukin-1 cluster. Genomics 79:718-725
Sims J E, Nicklin M J, Bazan J F, Barton J L, Busfield S J, Ford J E, Kastelein R A, Kumar S, Lin H, Mulero J J, Pan J, Pan Y, Smith D E, Young P R (2001) A new nomenclature for IL-1-family genes. Trends Immunol 22:536-537
Smith D E, Renshaw B R, Ketchem R R, Kubin M, Garka K E, Sims J E (2000) Four new members expand the interleukin-1 superfamily. J Biol Chem 275:1169-1175
Stephens M, Donnelly P (2003) A comparison of Bayesian methods for haplotype reconstruction from population genotype data. Am J Hum Genet 73:1162-1169
van der Linden S, Valkenburg H, Cats A (1983) The risk of developing ankylosing spondylitis in HLA-B27 positive individuals: a family and population study. Br J Rheumatol 22:18-19
(1984) Evaluation of diagnostic criteria for ankylosing spondylitis. A proposal for modification of the New York criteria. Arthritis Rheum 27:361-368
van der Paardt M, Crusius J B, Garcia-Gonzalez M A, Baudoin P, Kostense P J, Alizadeh B Z, Dijkmans B A, Pena A S, van der Horst-Bruinsma I E (2002) Interleukin-lbeta and interleukin-1 receptor antagonist gene polymorphisms in ankylosing spondylitis. Rheumatology (Oxford) 41:1419-1423
Vazquez-Del M M, Garcia-Gonzalez A, Munoz-Valle J F, Garcia-Iglesias T, Martinez-Bonilla G, Bemard-Medina G, Sanchez-Ortiz A, Omelas-Aguirre J M, Salazar-Paramo M, Gamez-Nava J I, Gonzalez-Lopez L (2002) Interleukin lbeta (IL-1beta), IL-10, tumor necrosis factor-alpha, and cellular proliferation index in peripheral blood mononuclear cells in patients with ankylosing spondylitis. J Rheumatol 29:522-526
   Equivalents

Those skilled in the art will recognize, or be able to ascertain using no more than routine experimentation, many equivalents of the specific embodiments of the invention described herein. Such equivalents are intended to be encompassed by the following claims.

TABLE 5
Pairwise Linkage Disequilibrium Values across the IL-1 Gene Cluster

| | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| 1 (IL-1A559) | | 0 | 0 | 0 | 0 | .25 | .53 | .26 | .22 | 0 | .14 | .05 | .08 | .08 | 0 |
| 2 (IL-1A376) | .81 | | 0 | 0 | .96 | .02 | .69 | .19 | .39 | 0 | 0 | 0 | 0 | .01 | 0 |
| 3 (IL-1A889) | .59 | .80 | | 0 | 0 | .31 | .95 | .42 | .20 | 0 | 0 | 0 | 0 | .37 | 0 |
| 4 (IL-1B3953) | .49 | .73 | .57 | | 0 | 0 | .01 | 0 | 0 | 0 | .01 | .01 | .01 | .02 | 0 |
| 5 (IL-1B5810) | .48 | 0 | .52 | .86 | | 0 | 0 | 0 | 0 | 0 | .02 | 0 | .01 | .15 | 0 |
| 6 (IL-1B-511) | .05 | .21 | .04 | .20 | .79 | | .11 | .24 | .29 | 0 | .59 | .11 | .29 | .79 | .01 |
| 7 (IL-1F7-1) | .05 | .08 | .01 | .21 | .68 | .14 | | 0 | 0 | .83 | .67 | .60 | .79 | 0 | .60 |
| 8 (IL-1F7-2) | .09 | .25 | .07 | .24 | .70 | .10 | .94 | | 0 | .31 | .50 | .87 | .94 | 0 | .48 |
| 9 (LOCUS 129359) | .11 | .18 | .12 | .30 | .59 | .10 | .83 | .90 | | .32 | .73 | .88 | .63 | 0 | .82 |
| 10 (IL-1F9-1) | .47 | .77 | .48 | .66 | .83 | .23 | .01 | .06 | .06 | | .73 | .86 | .54 | .12 | .73 |
| 11 (IL-1F6-1) | .06 | .76 | .16 | .11 | .20 | .02 | .03 | .05 | .03 | .05 | | 0 | 0 | 0 | 0 |
| 12 (IL-1F6-2) | .08 | .69 | .16 | .12 | .24 | .07 | .04 | .01 | .01 | .02 | .95 | | 0 | 0 | 0 |
| 13 (IL-1F6-3) | .08 | .73 | .18 | .11 | .22 | .05 | .02 | .01 | .04 | .09 | .94 | .94 | | 0 | 0 |
| 14 (IL-1F8-1) | .16 | .58 | .09 | .19 | .25 | .05 | .18 | .17 | .19 | .10 | .65 | .64 | .62 | | 0 |
| 15 (IL-1F8-2) | .13 | .74 | .18 | .15 | .27 | .11 | .05 | .06 | .02 | .02 | .70 | .68 | .73 | .65 | |
| 16 (IL-1F8-3) | .15 | .76 | .21 | .12 | .23 | .12 | .12 | .15 | .10 | .06 | .68 | .64 | .71 | .65 | .92 |
| 17 (IL-1F5-1) | .13 | .72 | .18 | .10 | .20 | .11 | .10 | .11 | .02 | .02 | .66 | .65 | .68 | .62 | .91 |
| 18 (IL-1F5-2) | .09 | .14 | .21 | .40 | .36 | .59 | .67 | .62 | .73 | .59 | .10 | .13 | .14 | .19 | .04 |
| 19 (IL-1F5-3) | .05 | .06 | .15 | .38 | .41 | .58 | .75 | .69 | .68 | .50 | .03 | .04 | .03 | 0 | .13 |
| 20 (IL-1F5-4) | .26 | .72 | .27 | .29 | .25 | .09 | .09 | .07 | .08 | .13 | .69 | .68 | .71 | .58 | .91 |
| 21 (IL-1F10-1) | .33 | .70 | .40 | .62 | .74 | .24 | .10 | .10 | .13 | .68 | .10 | .13 | .15 | .14 | .20 |
| 22 (IL-1F10-2) | .31 | .63 | .35 | .55 | .69 | .23 | .09 | .09 | .12 | .65 | .14 | .14 | .20 | .13 | .07 |
| 23 (IL-1F10-3) | .53 | .81 | .23 | .33 | .63 | .27 | .20 | .19 | .20 | .31 | .25 | .35 | .29 | .24 | .20 |
| 24 (IL-1RN8006) | .16 | .28 | .18 | .21 | .50 | .44 | .17 | .14 | .18 | .14 | .01 | .03 | .04 | .17 | .11 |
| 25 (IL-1RN8061) | .02 | .06 | .10 | .09 | .19 | .31 | .11 | .05 | .14 | .13 | .10 | .08 | .10 | .07 | .26 |
| 26 (IL-1RNVNTR) | .10 | .31 | .18 | .17 | .53 | .46 | .05 | .06 | .04 | .15 | .10 | .09 | .08 | .07 | .11 |
| 27 (IL-1RN9589) | .01 | .06 | .12 | .03 | .20 | .25 | .01 | 0 | .13 | .04 | .13 | .11 | .11 | .24 | .22 |
| 28 (IL-1RN11100) | .17 | .24 | .13 | .17 | .03 | .08 | .07 | .11 | .15 | .09 | .02 | .02 | .07 | .53 | .03 |

| | 16 | 17 | 18 | 19 | 20 | 21 | 22 | 23 | 24 | 25 | 26 | 27 | 28 |
|---|---|---|---|---|---|---|---|---|---|---|---|---|---|
| 1 (IL-1A559) | 0 | 0 | .23 | .46 | 0 | 0 | 0 | 0 | 0 | .57 | .01 | .91 | 0 |
| 2 (IL-1A376) | 0 | 0 | .01 | .30 | 0 | 0 | 0 | 0 | .02 | .13 | 0 | .58 | .02 |
| 3 (IL-1A889) | 0 | 0 | 0 | .02 | 0 | 0 | 0 | .04 | 0 | .03 | 0 | .01 | 0 |
| 4 (IL-1B3953) | .01 | .02 | 0 | 0 | 0 | 0 | 0 | 0 | 0 | .02 | 0 | .42 | 0 |
| 5 (IL-1B5810) | 0 | .01 | 0 | 0 | 0 | 0 | 0 | 0 | 0 | .02 | 0 | .02 | .48 |
| 6 (IL-1B-511) | 0 | .01 | 0 | 0 | .04 | 0 | 0 | .01 | 0 | 0 | 0 | 0 | .35 |
| 7 (IL-1F7-1) | .17 | .26 | 0 | 0 | .37 | .03 | .05 | 0 | .01 | .17 | .49 | .93 | .39 |
| 8 (IL-2F7-2) | .07 | .19 | 0 | 0 | .47 | .02 | .04 | 0 | .03 | .48 | .40 | .96 | .17 |
| 9 (LOCUS 129359) | .26 | .80 | 0 | 0 | .46 | .01 | .02 | 0 | .01 | .10 | .55 | .52 | .08 |
| 10 (IL-1F9-1) | .33 | .70 | 0 | 0 | .06 | 0 | 0 | 0 | 0 | .01 | .01 | .45 | .09 |
| 11 (IL-1F6-1) | 0 | 0 | .21 | .66 | 0 | .13 | .04 | .01 | .78 | .32 | .02 | .19 | .84 |
| 12 (IL-1F6-2) | 0 | 0 | .08 | .56 | 0 | .07 | .04 | 0 | .80 | .43 | .02 | .28 | .83 |
| 13 (IL-1F6-3) | 0 | 0 | .07 | .66 | 0 | .03 | 0 | 0 | .74 | .35 | .03 | .30 | .48 |
| 14 (IL-1F8-1) | 0 | 0 | .23 | .98 | 0 | .01 | .01 | 0 | .02 | .38 | 0 | .27 | 0 |
| 15 (IL-1F8-2) | 0 | 0 | .45 | .01 | 0 | .21 | .66 | .07 | .32 | .01 | .01 | .02 | .48 |
| 16 (IL-1F8-3) | | 0 | .39 | 0 | 0 | .45 | .83 | .03 | .30 | .01 | .01 | .06 | .93 |
| 17 (IL-1F5-1) | .89 | | .13 | 0 | 0 | .04 | .09 | .28 | .34 | 0 | .03 | .02 | .57 |
| 18 (IL-1F5-2) | .04 | .07 | | 0 | .59 | 0 | 0 | .14 | 0 | 0 | 0 | .06 | .25 |
| 19 (IL-1F5-3) | .14 | .18 | .80 | | .01 | 0 | 0 | .15 | 0 | 0 | 0 | .03 | .05 |
| 20 (IL-1F5-4) | .85 | .90 | .02 | .12 | | .18 | .38 | .10 | .37 | .01 | .05 | .01 | 0 |
| 21 (IL-1F10-1) | .12 | .32 | .90 | .93 | .19 | | 0 | 0 | .02 | .66 | .93 | .71 | 0 |
| 22 (IL-1F10-2) | .03 | .26 | .91 | .88 | .12 | .97 | | 0 | .06 | .61 | .88 | .66 | 0 |
| 23 (IL-1F10-3) | .24 | .12 | .28 | .28 | .21 | .24 | .23 | | 0 | 0 | .01 | 0 | .05 |
| 24 (IL-1RN8006) | .11 | .10 | .47 | .57 | .08 | .14 | .11 | .48 | | 0 | 0 | 0 | .09 |
| 25 (IL-1RN8061) | .24 | .31 | .40 | .51 | .20 | .03 | .03 | .36 | .62 | | 0 | 0 | .35 |
| 26 (IL-1RNVNTR) | .12 | .08 | .63 | .75 | .11 | .02 | .03 | .30 | .74 | .52 | | 0 | 0 |
| 27 (IL-1RN9589) | .17 | .21 | .14 | .16 | .20 | .03 | .03 | .40 | .44 | .45 | .33 | | .03 |
| 28 (IL-1RN11100) | .01 | .03 | .06 | .10 | .21 | .21 | .20 | .20 | .20 | .09 | .32 | .22 | |

NOTE
Numbers (1-28) correspond to the same marker names in both the columns and rows. Lewontin's standardized linkage disequilibrium coefficient is given in the bottom left half of the table, and the corresponding P values are given in the upper right of the table. P values < .001 are reported as P = 0.

1. An isolated polypeptide of at least 50 amino acids comprising an amino acid sequence which is at least 70% identical to a polypeptide sequence set forth in SEQ ID No. 5 or 6.

2-11. (canceled)

12. An isolated nucleic acid comprising a nucleotide sequence which is at least 70% identical to the entire nucleotide sequence set forth in SEQ ID No. 1 or 4 or complement thereof.

13-24. (canceled)

25. A method of identifying a subject suffering from or at risk of developing ankylosing spondylitis, comprising the step of identifying an IL-1L1 allele in a biological sample obtained from said subject, wherein the presence of said IL-1L1 allele indicates that said subject is suffering from or is at risk of developing ankylosing spondylitis.
